SEQUENCE LISTING (8 sequences)

SEQ ID NO: 1 - DNA, 20 bp, Artificial Sequence
  Primer
attgtggaccagaccagacc

SEQ ID NO: 2 - DNA, 20 bp, Artificial Sequence
  Primer
gctggtgaggcactttcttc

SEQ ID NO: 3 - DNA, 20 bp, Artificial Sequence
  Primer
atggcgagtagcagcggctc

SEQ ID NO: 4 - DNA, 20 bp, Artificial Sequence
  Primer
cgtatgtgggggatgccaac

SEQ ID NO: 5 - DNA, 20 bp, Artificial Sequence
  Primer
agaagttggtggagctgtcg

SEQ ID NO: 6 - DNA, 20 bp, Artificial Sequence
  Primer
cagctgcttgaagaactgcc

SEQ ID NO: 7 - DNA, 20 bp, Artificial Sequence
  Primer
gtttcaatctgcggctgaca

SEQ ID NO: 8 - DNA, 20 bp, Artificial Sequence
  Primer
gaagagcattctgccgcttg

(19) United States
(12) Patent Application Publication
(10) Pub. No.: US 2023/0055209 A1
(43) Pub. Date: Feb. 23, 2023

(54) LON PROTEASE, ALPHA-HEMOLYSIN, CK1-ALPHA-1; C-MYB INHIBITOR OR A CEBP-DELTA INHIBITOR AS THERAPEUTICS

(71) Applicant: LINNANE PHARMA AB, Malmö (SE)

(72) Inventors: Catharina SVANBORG, Malmö (SE); Daniel Sebastian Christopher BUTLER, Lund (SE)

(73) Assignee: LINNANE PHARMA AB, Malmö (SE)

(21) Appl. No.: 17/786,256

(22) PCT Filed: Dec. 18, 2020
(86) PCT No.: PCT/EP2020/087245

§ 371 Date: Jun. 16, 2022

(30) Foreign Application Priority Data
Dec. 20, 2019 (GB) 1919036.2
Nov. 6, 2020 (GB) 2017598.0

(51) Int. Cl. C12N 9/52; C12N 9/12; C07K 14/31; A61P 35/00; A61P 13/10; A61P 13/02; A61P 31/04; C07K 14/245

(52) U.S. Cl. CPC C12N 9/52; C12N 9/12; C07K 14/31; A61P 35/00; A61P 13/10; A61P 13/02; A61P 31/04; C07K 14/245; A61K 38/00

(57) ABSTRACT

A therapeutic agent comprising Lon protease, or a variant or active fragment thereof, alpha-hemolysin, or a variant or active fragment thereof, CK1α1, or a variant or active fragment thereof, a c-MYB inhibitor and/or a CEBP-δ inhibitor, for use in therapy, with the proviso that the therapeutic agent does not comprise a bacteria or bacterial supernatant. Methods of production and use thereof.

### TECHNICAL FIELD

This invention relates to entities for suppressing MYC activity, particularly Lon protease, alpha-hemolysin, CK1α1, c-MYB inhibitors and CEBP-δ inhibitors, as well as to methods of manufacture and use of these entities.

### BACKGROUND

The transcriptional machinery is fine-tuned to optimize the survival of all classes of plant and animal life. Balance is maintained by exquisitely regulated molecular interactions between specific transcription factors, enhancers and repressors of specific genes or gene families. In addition, gene expression is classically controlled by the pleiotropic transcription factors that determine the overall activity of large numbers of genes and thereby the character and consistency of gene expression. These include the MYC family, which regulates cellular growth and development. c-MYC is deregulated in the majority of all human cancers and its ability to bind to a variety of promoters contributes to the broad transforming effect. c-MYC has also been associated with oncogenic transformation in malignancies driven by chronic infection, regulated through direct interactions of NF-κB, STAT1, STAT4, c-Jun and c-Fos with the c-MYC promoter.

The affinity of MYC for a vast number of promoters contributes to its broad transforming effects and c-MYC deregulation is associated with a poor prognosis in the majority of human cancers. MYC inhibition would therefore be a desirable approach to cancer therapy, but despite massive efforts, targeting of MYC itself has been unsuccessful, owing to its “undruggable” protein structure⁸. Attempts have also been made to target the MYC transcription machinery through for example cofactors such as Max, CDKs and BRD4, USPs or PLK1, with limited success.

The MYC transcription factors define essential aspects of renal development, such as the fusion of ectoderm with endoderm and the subsequent differentiation of the renal cortex and medulla. Kidney infections often impair renal growth, suggesting that MYC might be targeted. In a pilot study, the inventors identified c-MYC as an active transcriptional node in children susceptible to kidney infections. MYC, MYCN, KLF2, PAX5 and MAF were activated, the MYC-MAX motif was strongly enriched and >60% of downstream genes had an expression pattern consistent with MYC activation (FIG. 5). In contrast, MYC was not regulated in children with asymptomatic bacteriuria (ABU) (Tables 1, 2 and FIG. 5). The inventors have previously demonstrated whole bacteria and bacterial supernatants that inhibit c-MYC activity (WO 2018/069886).

The inventors further show that uropathogenic Escherichia coli bacteria regulate the host environment by directly degrading c-MYC and inhibiting Myc expression in infected tissues.

We provide a molecular mechanism for this effect and demonstrate therapeutic efficacy against cancer, in several animal models. The results suggest that molecules of bacterial origin may offer a new approach to solving the classical problem of therapeutic MYC inhibition.

### SUMMARY OF THE INVENTION

Through extensive efforts, the inventors have identified unexpected entities that inhibit c-MYC activity, through enhancing the degradation of c-MYC and inhibiting expression of c-MYC. In particular, Lon protease, alpha-hemolysin and CK1α1 have been identified as entities that enhance degradation of c-MYC. c-MYB and CEBP-δ have been identified as transcription factors in the host cell that are responsible for enhanced c-MYC expression.

Lon protease is an ATP-dependent serine peptidase. The inventors have identified that Lon protease is secreted into the bacterial supernatant and successfully invades the cytoplasm and nucleoplasm of the host cell. Furthermore, the Lon protease crosses these multiple membranes and passes through a variety of different biological environments while retaining activity. Even more surprisingly, the Lon protease was found to degrade c-Myc, and this degradation was found to be fast enough and widespread enough to have a useful biological impact.

The inventors also identified that alpha-hemolysin is secreted into the bacterial supernatant. Alpha-hemolysin is a toxin that bacteria typically use to lyse red blood cells through destruction of the red blood cell membrane. It is therefore particularly surprising that alpha-hemolysin was observed to upregulate expression of the host cell CK1α1 kinase. It has also been identified that CK1α1 phosphorylates c-MYC at serine 252 thereby marking the protein for proteasomal degradation. Overall, therefore, alpha-hemolysin and CK1α1 have the surprising and unexpected effect of marking c-MYC for degradation.

The inventors have also made the unexpected discovery that c-MYB and CEBP-δ within the host cell are transcription factors that regulate c-MYC expression. Surprisingly, the inventors observed bacteria that secreted an inhibitor of c-MYB and CEBP-δ expression, thus reducing c-MYC expression in the host cell.

As such, a first aspect of the invention provides a therapeutic agent comprising Lon protease, or a variant or active fragment or equivalent thereof, alpha-hemolysin, or a variant or active fragment or equivalent thereof, CK1α1, or a variant or active fragment or equivalent thereof, a c-MYB inhibitor and/or a CEBP-δ inhibitor, for use in therapy, with the proviso that the therapeutic agent does not comprise a bacteria or bacterial supernatant or lysate. In a preferred embodiment, the therapeutic agent comprises Lon protease, or a variant or active fragment or equivalent thereof, and/or alpha-hemolysin, or a variant or active fragment or equivalent thereof. In a preferred embodiment, the therapeutic agent comprises both a c-MYB inhibitor and a CEBP-δ inhibitor, as these inhibitors potentially cooperate to inhibit c-MYC. In a particularly preferred embodiment, the therapeutic agent comprises Lon protease, or a variant or active fragment or equivalent thereof.

In an embodiment, the invention provides a therapeutic agent comprising Lon protease, or a variant or active fragment thereof, alpha-hemolysin, or a variant or active fragment thereof, CK1α1, or a variant or active fragment thereof, a c-MYB inhibitor and/or a CEBP-δ inhibitor, for use in therapy, with the proviso that the therapeutic agent does not comprise a bacteria or bacterial supernatant or lysate. In a preferred embodiment, the therapeutic agent comprises Lon protease, or a variant or active fragment thereof, and/or alpha-hemolysin, or a variant or active fragment thereof. In a particularly preferred embodiment, the therapeutic agent comprises Lon protease, or a variant or active fragment thereof.

As used herein, the expression ‘variant’ refers to a peptide sequence in which the amino acid sequence differs from the basic protein or peptide sequence in that one or more amino acids within the sequence are substituted for other amino acids. However, the variant produces a biological effect which is similar to that of the basic sequence, i.e. it is active.

In an embodiment, the length of the variant is generally at least 50%, 55%, 60%, 65%, 70%, 75%, 80%, 85%, 90% or 95% of the length of the original sequence. In the same or another embodiment, the length of the variant is generally less than 200%, 190%, 180%, 170%, 160% or 150% of the length of the original sequence.

In an embodiment, the variant is generally at least 10, 20, 30, 40 or 50 amino acids in length.

Amino acid substitutions may be regarded as “conservative” where an amino acid is replaced with a different amino acid in the same class with broadly similar properties. Non-conservative substitutions are where amino acids are replaced with amino acids of a different type or class.

Amino acid classes are defined as follows:

Class Amino acid examples

Nonpolar: A, V, L, I, P, M, F, W

Uncharged polar: G, S, T, C, Y, N, Q

Acidic: D, E

Basic: K, R, H.

As is well known to those skilled in the art, altering the primary structure of a peptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptide's conformation.

Non-conservative substitutions may also be possible provided that these do not interrupt the function of the protein or peptide. Broadly speaking, fewer non-conservative substitutions will be possible without altering the biological activity of the polypeptides.

In general, variants will have amino acid sequences that will be at least 70%, for instance at least 71%, 75%, 79%, 81%, 84%, 87%, 90%, 93% or 96% identical to the basic sequence. Identity in this context may be determined using the BLASTP computer program with the basic native protein sequence as the base sequence. The BLAST software is publicly available at http://blast.ncbi.nlm.nih.gov/Blast.cgi (accessible on 12 Mar. 2009).

Variants may also include additional sequences such as tag sequences that may be used for instance in facilitating purification of the peptide or in detection of it. Thus for instance, the variant may further comprise an affinity tag such as chitin binding protein (CBP), maltose binding protein (MBP), glutathione-S-transferase (GST), FLAG, myc, biotin or a poly(His) tag as are known in the art. In another embodiment, the variant may comprise a fluorescent protein such as green fluorescent protein (GFP).

As used herein, the term ‘active fragment’ refers to any portion of the given amino acid sequence which still shows therapeutic (i.e. MYC inhibitory) activity. Fragments may comprise more than one portion from within the full-length protein, joined together. Portions will suitably comprise at least 5 and preferably at least 10 consecutive amino acids from the basic sequence. Suitable fragments will include deletion mutants comprising at least 10 amino acids, for instance at least 20, more suitably at least 50 amino acids in length or analogous synthetic peptides with similar structures. They include small regions from the protein or combinations of these.

As used herein, the term ‘equivalent’ refers to an effector molecule that is capable of reproducing one or more effects of the parent molecule in a mammalian system. The inventors have established that the parent molecules have an effect on gene regulation in human cells, up-regulating and down-regulating specific genes. The technical effect of the invention may be reproduced by effector molecules that replicate the gene regulatory activity. For instance, the effector molecule may be capable of reproducing one or more effects produced by Lon protease in a mammalian, preferably human, cell. In one embodiment, the effector molecule is capable of reproducing C-Myc cleaving protease activity of Lon protease. In one embodiment, the effector molecule is capable of reproducing one or more gene regulation effects produced by Lon protease in a mammalian, preferably human, cell. Preferred effects for replication by an effector molecule are set out in further detail herein.

As used herein, the expression ‘a c-MYB inhibitor’ or ‘a CEBP-δ inhibitor’ refers to any entity which controls, regulates or limits the activity of c-MYB or CEBP-δ, in particular in vivo in mammals. This may be achieved for example by degradation of c-MYB or CEBP-δ, downregulation of expression of c-MYB or CEBP-δ, and/or direct inhibition of c-MYB or CEBP-δ binding interactions, particularly at sites involved in the C-MYC transcription regulation pathway.

The therapeutic agent does not comprise a bacterial supernatant. The meaning of ‘bacterial supernatant’ will be readily understood by the skilled person. For the avoidance of doubt, this term refers to the culture media into which the bacteria secretes molecules. The bacterial supernatant may undergo simple workup steps, such as centrifugation and filtration to remove bacterial cells and cell debris, and still be referred to as a bacterial supernatant.

In one embodiment, the therapeutic agent does not comprise a microbe or microbial supernatant or lysate.

In a preferred embodiment, the Lon protease, alpha-hemolysin, c-MYB inhibitor and/or CEBP-δ inhibitor are bacterial. By this, we mean that these molecules have a structure that is of bacterial origin, and therefore covers synthetic reproductions of molecules of bacterial origin. In the case of proteins, for example, the proteins could be biosynthesized by the bacteria, biosynthesized recombinantly as exogenous molecules in an artificial expression system or could be artificial synthetic proteins that match the bacterial protein sequence. Inhibitors may be obtainable from a wide range of bacterial species, including for example, Streptococcus, Pseudomonas, Shigella, Campylobacter, and Salmonella species. In a particular embodiment the bacteria is uropathogenic bacteria, preferably E. coli, more preferably E. coli 536 or CFT073.

The therapeutic agent can comprise isolated Lon protease, or a variant or active fragment thereof, isolated alpha-hemolysin, or a variant or active fragment thereof, isolated CK1α1, or a variant or active fragment thereof, an isolated c-MYB inhibitor and/or an isolated CEBP-δ inhibitor. By ‘isolated’, we mean that the entity has been isolated, in particular, from a microbial supernatant or from a mixture of reactants and byproducts of protein synthesis. The isolated agent can, of course, be formulated with further components, including but not limited to carriers, stabilisers, excipients and adjuvants. Where the therapeutic agent comprises two or more of the listed entities, those entities can be isolated separately from each other and combined to form the therapeutic agent or can be isolated together as the combination of those entities.

The therapeutic agent can comprise synthetic Lon protease, or a variant or active fragment thereof, synthetic alpha-hemolysin, or a variant or active fragment thereof, synthetic CK1α1, or a variant or active fragment thereof, a synthetic c-MYB inhibitor and/or a synthetic CEBP-δ inhibitor. In other words, the entities comprising the therapeutic agent can be made without the need for microbial expression. In one embodiment, the therapeutic agent can comprise a mixture of biosynthetically expressed and chemically synthesised entities.

In one embodiment, the c-MYB inhibitor and/or CEBP-δ inhibitor are proteins or variants or active fragments thereof. The c-MYB inhibitor and/or a CEBP-δ inhibitor can be inhibitors of c-MYB and/or CEBP-δ expression.

The therapeutic use is preferably treatment or prevention of a disease wherein c-MYC levels are elevated. In particular, the disease in which MYC levels are elevated and which is therefore susceptible to prevention or treatment is cancer. Examples of such cancers may include lymphoma, cervical cancer, colon cancer, breast cancer, lung cancer, small airway cancer, stomach cancer, kidney cancer, bladder cancer, bowel cancer, mouth cancer or cancer of the gastrointestinal track. In a particular embodiment, the cancer may be a lymphoma or associated with a mucosal or epithelial surface, such as Burkitt lymphoma, lung cancer, kidney cancer, bladder cancer, colon cancer, bowel cancer, mouth cancer or cancer of the gastrointestinal track.

It has also been identified that c-MYC inhibitors can be used in the treatment of infection (see, for example, WO 2018/069886). Examples of such infection include infection of the urinary tract, preferably of the bladder and/or kidney. The infections may be, for example, bacterial or viral, especially bacterial infections.

In a particular embodiment, the therapeutic agent comprises Lon protease, or a variant or active fragment thereof. The therapeutic agent can comprise both Lon protease, or a variant or active fragment thereof, and alpha-hemolysin, or a variant or active fragment thereof. In one embodiment, the therapeutic agent further comprises CK1α1, or a variant or active fragment thereof. In one embodiment, the therapeutic agent further comprises a c-MYB inhibitor and a CEBP-δ inhibitor. Where the therapeutic agent comprises two or more entities for use in conjunction with each other, the therapeutic agent will generally have an enhanced ability to inhibit c-MYC. Where the therapeutic agent comprises two or more entities for use in conjunction, the entities can be administered in combination, simultaneously or sequentially.

According to a second aspect, the invention provides a recombinant microorganism engineered to express elevated levels of Lon protease, alpha-hemolysin, CK1α1, c-MYB inhibitor, and/or CEBP-δ inhibitor. In an embodiment, the microorganism is engineered to express elevated levels of Lon protease. In an embodiment, the microorganism is engineered to express elevated levels of alpha-hemolysin. In an embodiment, the microorganism is engineered to express elevated levels of CK1α1. In an embodiment, the microorganism is engineered to express elevated levels of a c-MYB inhibitor. In an embodiment, the microorganism is engineered to express elevated levels of EBP-δ inhibitor.

A third aspects provides a microorganism extract obtained from the recombinant microorganism of the second aspect of the invention. The extract can be a supernatant or a product of further purification of the supernatant. The microorganism can be a bacteria or fungus, such as yeast, specifically used for high-level protein expression. In this case is the expression products would typically be purified from the microorganism before therapeutic use.

According to a fourth aspect, the invention provides a method of obtaining isolated Lon protease, or a variant or active fragment thereof, isolated alpha-hemolysin, or a variant or active fragment thereof, isolated CK1α1, or a variant or active fragment thereof, an isolated c-MYB inhibitor and/or an isolated CEBP-δ inhibitor, the method comprising isolating the Lon protease, or a variant or active fragment thereof, the alpha-hemolysin, or a variant or active fragment thereof, the CK1α1, or a variant or active fragment thereof, the c-MYB inhibitor and/or the CEBP-δ inhibitor from the recombinant microorganism of the second aspect of the invention or the microorganism extract of the third aspect of the invention.

According to a fifth aspect, the invention provides a kit comprising isolated Lon protease, or a variant or active fragment thereof, isolated alpha-hemolysin, or a variant or active fragment thereof, isolated CK1α1, or a variant or active fragment thereof, an isolated c-MYB inhibitor and/or an isolated CEBP-δ inhibitor. The kit can comprise synthetic Lon protease, or a variant or active fragment thereof, synthetic alpha-hemolysin, or a variant or active fragment thereof, synthetic CK1α1, or a variant or active fragment thereof, a synthetic c-MYB inhibitor and/or a synthetic CEBP-δ inhibitor. The Lon protease, or variant or active fragment thereof, alpha-hemolysin, or variant or active fragment thereof, CK1α1, or a variant or active fragment thereof, c-MYB inhibitor and/or CEBP-δ inhibitor can be in powder form. For example, they can be lyophilized or otherwise dehydrated into powder form.

According to a sixth aspect, the invention provides a recombinant microorganism according to the second aspect, a recombinant microorganism extract according to the third aspect, or a kit according to the fifth aspect, for use in therapy.

According to a seventh aspect, the invention provides a pharmaceutical composition, comprising a therapeutic agent according to the first aspect, a recombinant microorganism according to the second aspect, or a microorganism extract according to the third aspect, plus a pharmaceutically acceptable carrier, excipient and/or adjuvant. Suitable pharmaceutical compositions will be in either solid or liquid form. They may be adapted for administration by any convenient route, such as parenteral, oral or topical administration or for administration by inhalation or insufflation. The pharmaceutical acceptable carrier may include diluents or excipients which are physiologically tolerable and compatible with the active ingredient.

Parenteral compositions are prepared for injection, for example either subcutaneously or intravenously. They may be liquid solutions or suspensions, or they may be in the form of a solid that is suitable for solution in, or suspension in, liquid prior to injection. Suitable diluents and excipients are, for example, water, saline, dextrose, glycerol, or the like, and combinations thereof. In addition, if desired the compositions may contain minor amounts of auxiliary substances such as wetting or emulsifying agents, stabilizing or pH-buffering agents, and the like.

Oral formulations will be in the form of solids or liquids, and may be solutions, syrups, suspensions, tablets, pills, capsules, sustained-release formulations, or powders. Oral formulations include such normally employed excipients as, for example, pharmaceutical grades of mannitol, lactose, starch, magnesium stearate, sodium saccharin, cellulose, magnesium carbonate, and the like.

Topical formulations will generally take the form of suppositories or intranasal aerosols. For suppositories, traditional binders and excipients may include, for example, polyalkylene glycols or triglycerides; such suppositories may be formed from mixtures containing the active ingredient.

According to an eighth aspect, the invention provides a method of treating cancer, the method comprising administering an effective amount of a therapeutic agent according to any of claims 1-8, a recombinant microorganism according to claim 9, a microorganism extract according to claim 10, or a pharmaceutical composition according to claim 16, to a patient in need thereof.

The amount of inhibitor administered will vary in accordance with normal clinical practice, and will depend upon factors such as the nature of the reagent being used, the size and health of the patient, the nature of the condition being treated etc. in accordance with normal clinical practice. Typically, a dosage in the range of from 1 μg-50 mg/kg for instance from 2-20 mg/kg, such as from 5-15 mg/kg would be expected to produce a suitable effect.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1 shows c-MYC suppression by uropathogenic E. coli strains: (A) Decrease in c-MYC protein levels after infection with the uropathogenic E. coli strain CFT073 (10⁵ CFU/ml, 1 or 4 hours). Quantification of fluorescence in human kidney cells (n=5, P<0.01 compared to uninfected cells). Green=c-MYC; blue=nuclei. Scale bar, 10 μm. (B) Western blot analysis of c-MYC in whole cell extracts confirming the reduction in (A) (P<0.01, 4 hours, n=5). (C) c-MYC expression in cells infected with acute pyelonephritis (APN) (n=36) or asymptomatic bacteriuria (ABU) (n=20) isolates (10⁵ CFU/ml, 4 hours). Western blot analysis of whole cell extracts, compared to uninfected cells (n=2 per strain, P<0.001). (D) Reduction in renal pelvic c-MYC staining (green) 24 hours after infection with CFT073 or E. coli 536 (10⁸ CFU/ml, 24 hours, n=4 mice per group), (P<0.01 compared to uninfected controls). (E) MYC related genes identified by transcriptomic analysis of E. coli 536 infected kidneys. Ten most up- or down-regulated genes are shown. Means±S.E.M, Mann-Whitney U-test, *P<0.05, **P<0.01, ***P<0.001. (F) Schematic representation of the E. coli 536 chromosome, indicating the positions of Pathogenicity Islands (PAIs) I-V. (G) E. coli 536 mutants (ΔPAI I to ΔPAI V and ΔPAI I+II) were screened for effects on cellular c-MYC levels (4 hours). c-MYC inhibition was regulated by genes on PAI I. Western blot analysis of whole cell extracts. (H) Schematic illustrating the PAI I gene deletion strategy used to identify MYC regulatory genes. (I) Loss of MYC inhibition in the E. coli 536 PAI IΔ2.5.3 sub-clone. The deleted gene cluster encodes a metal ion ABC transporter. (J) ZnCl₂ pretreatment of E. coli 536 inhibits bacterial MYC degradation. (K) c-MYC is also inhibited by E. coli 536 supernatant. The addition of ZnCl₂ to the active supernatant did not impair its effect on host cells Data is represented as means±S.E.M, *P<0.05, **P<0.01, ***P<0.001, Paired t-test.

FIG. 2 shows data relating to the mechanism of accelerated c-MYC degradation: (A) Screen for c-MYC inhibitors in bacterial culture supernatants, using LC-MS/MS mass spectrometry. Lon protease and hemolysin were identified as the most abundant proteins. (B) Recombinant Lon protease degraded c-MYC in vitro, in a time-dependent manner. (C) Co-immunoprecipitation of Lon protease with c-MYC in E. coli 536 supernatant indicating binding. (D, E) Dose-dependent Lon uptake (red) and MYC degradation (green) in human kidney cells (4 hours). n=3 experiments, (F) Genes involved in proteasome degradation, including CSNK1A1 and PLK3. CSNK1A1 was upregulated in E. coli 536 infected cells but not by ΔPAI I (qRT-PCR, FIG. 9). CK1α1 phosphorylates c-MYC at Serine²⁵², marking the protein for proteosomal degradation. (G, H) CK1α1 activation, shown by a redistribution from the nuclei to characteristic cytoplasmic puncta. Green=CK1α1; blue=nuclei. Scale bar, 10 μm (n=3 experiments). (I) Co-immunoprecipitation of CK1α1 and c-MYC in vitro, using recombinant c-MYC and lysates from infected cells (n=3 experiments). (J-I) Reversal of c-MYC inhibition by the CK1α1 inhibitor IC261 (30 minutes). Green=c-MYC; blue=nuclei. Scale bar, 10 μm. Data is represented as means±S.E.M, *P<0.05, **P<0.01, ***P<0.001, Paired t-test.

FIG. 3 shows data demonstrating that MYC expression was inhibited by infection and transcriptional MYC regulators were affected: (A) MYC expression was further inhibited by E. coli 536 infection but not by E. coli 536 ΔPAI I or PAI IΔ2.5.3 (qRT-PCR, 4 hours, n=4 experiments). (B) MAX protein levels, in contrast, remained intact and MAX expression was not affected (qRT-PCR, 4 hours n=3, see transcriptomic analysis in FIG. 12) (C) Transcriptomic analysis identified genes inversely regulated by E. coli 536 and ΔPAI I (Heat-map) including (D) a group of MYB-related genes (CEBPD, BIRC3 and PTGS2). (E) CEBPD expression was inhibited in cells infected with E. coli 536 but not ΔPAI I (qRT-PCR, 4 hours, n=4 experiments) and CEBP-δ protein levels were reduced both by infection and by E. coli 536 supernatant. Green=CEBP-δ; blue=nuclei. Scale bar, 10 μm. (F) c-MYB expression was not affected but protein levels were reduced by E. coli 536. (G-H) The CK1α1 inhibitor IC261 reversed c-MYB inhibition by E. coli 536 (30 minutes). Green=c-MYB; blue=nuclei. Scale bar, 10 μm. (I) Model of c-MYC regulation by bacteria via a combination of accelerated degradation and inhibition of gene expression. Degradation was executed by the bacterial Lon protease and by the hly-dependent activation of CK1α1, in a Ca²⁺ dependent manner, targeting c-MYC and c-MYB for proteasomal degradation. Finally, we speculate that transcriptional repression of CEPBD and c-MYB degradation cooperate to inhibit the transcription of MYC. Data is represented as means±S.E.M. *P<0.05, **P<0.01, paired t-test.

FIG. 4 shows anti-tumorigenic effects of bacterial supernatants in vitro and in a bladder cancer model: (A) Broad c-MYC inhibition in E. coli 536 infected Burkitt lymphoma cells (Daudi and Raji), human and mouse bladder carcinoma (HTB-9 and MB49), lung carcinoma (A549), gastric (AGS), colon (DLD1) and hepatic (HEP-G2) carcinoma cells. (B) MYC inhibition by E. coli 536 supernatant in HTB9, A549, DLD1 and MB49 cells. ΔPAI I supernatant served as a negative control. Western blot analysis (n=3 experiments per cell type). (C) MB49 bladder tumors were established in C57BL/6 mice (see Supplemental FIG. 8). Tumor development was delayed in mice receiving E. coli 536 supernatant. Reduced bladder size and (D) tumor size, quantified from whole bladder frozen tissue sections (***P<0.001, comparing sham treated to E. coli 536 supernatant treated mice). Representative images from two mice. (E) Reduced c-MYC expression in mice treated with E. coli 536 supernatant compared to mice receiving PBS or ΔPAI I supernatant. (F) Confirmation of data in (E) by Western blot analysis of whole bladder tissue extracts (n=5 mice per group). (G) Transcriptomic analysis of whole bladder RNA. Principal component analysis demonstrated a significant shift from a disease-specific—towards a healthy tissue profile. (H) Quantification of bladder weight, bladder size, tumor size and MYC staining. Scatter plots indicate medians, Mann-Whitney U-test.

FIG. 5 shows data demonstrating elevated MYC expression in children prone to acute pyelonephritis (APN): Sequencing of peripheral blood RNA purified from children with APN or primary ABU, during an infection free interval (>1 year). (A) Heat map showing increased number of upregulated genes (red) in the APN group. (B) Table of regulated genes in individual patients. (C) Increased number regulated genes in the APN compared to the primary ABU group. (***P<0.001; χ²-test) (D) Canonical pathway analysis identified genes involved in transcriptional regulation, cell growth and functions. The corresponding genes were downregulated in the primary ABU group. (E) Activation of MYC-related gene networks in patients APN6 and ABU7. IPA network analysis, cut off FC 2.0. (F) Gene set enrichment analysis (GSEA)-plot identifying activated MYC/MAX transcriptional motifs in APN6 and inhibited motifs in ABU7 (ES=0.47, nominal P value, P<0.001).

FIG. 6 shows the MYC dependent gene network in infected kidneys: Mice were infected with the uropathogenic E. coli strain 536 and kidneys were harvested after 24 hours. A network of MYC related genes were affected, including 71 genes that were inhibited following infection (cut-off FC=1.41, compared to uninfected mice).

FIG. 7 shows data relating to the identification of the bacterial MYC inhibitor using a systematic gene deletion strategy in E. coli 536: (A) E. coli 536 mutants deleted for PAI I, PAI II or PAI I+PAI II-PAI were examined for effects on cellular C-MYC levels after infection of human kidney cells. Genes located on E. coli 536 PAI I were identified as MYC regulators. In addition, Δhly1 and ΔleuX showed partial effects, as well as Δhly2, located on PAI II. (B) Sequential deletion mutants from the 5′ end of PAI I E. coli 536 ΔPAI I were examined for effects on cellular c-MYC levels, as indicated in FIG. 2H. Bacterial MYC inhibition was controlled, in part, by a gene deleted in PAI IΔ2, encoding a bacterial ABC-transporter (C). PAI IΔ2.5.3, identified as a loss of function mutant in FIG. 2I was identified. Three ABC transporters (ABC1-3) were sequentially deleted from the CFT073 genome. The PAI IΔABC3 mutant showed loss of c-MYC inhibition. (D) ZnCl₂ treatment of the active E. coli 536 bacterial supernatant did not affect MYC levels in host cells, indicating that the ABC-transporter controls the expression and the release of the bacterial MYC inhibitor but not the effect on host cells.

FIG. 8 shows analysis of the bacterial proteins in supernatants of E. coli 536 and ΔPAI I: (A) Comparative analysis of supernatants from E. coli 536 and ΔPAI I. Five bands were abundant in E. coli 536 supernatant (bands 1-5, Coomassie staining). The bands were excised, processed by in-gel digestion and analyzed by LC-MS/MS. (B) Abundant constituents are listed. (C) The 20 most abundant protein categories in E. coli 536 supernatant compared ΔPAI I (Log 2 FC, Protein function database). (D) Protease digestion of the E. coli 536 supernatant removed the inhibitory activity, confirming that the soluble bacterial MYC inhibitor is a protein. Supernatants were pre-treated with increasing concentrations of trypsin (0-5 μg/ml, 30 minutes) before being added to A498 cells (4 hours, western blot analysis). Trypsin was used as a negative control.

FIG. 9 shows Lon dependent c-MYC degradation: (A) Kinetics of Lon dependent c-MYC degradation. Recombinant c-MYC (1 μg) was incubated with Lon protease (2 μg) at 37° C. for 0 to 4 hours, c-MYC alone was used as control (also in FIG. 2B). Accelerated, time-dependent c-MYC degradation, in the presence of Lon protease (from 200 ng to 79 ng, n=3 experiments, **P<0.01). (B) Relative Lon protease resistance of c-MYB (0-8 μg/ml, 4 hours) except the highest dose (45% decrease compared to PBS). (C) Lon protease resistance of cellular CEBP-δ levels (0-8 μg/ml, 4 hours) showing no CEBP-δ degradation (<5%, n=2 experiments).

FIG. 10 shows data demonstrating that hemolysin activates CK1α1 and enhances MYC degradation: CK1α1 is a constituent of the Wnt signalling pathway but can also be activated independently, by an ion flux-dependent mechanism. Hemolysin, which was the most abundant constituent of active bacterial supernatants, has been shown to activate Ca²⁺ fluxes in host cells. We investigated if hemolysin is involved in CK1α1 activation. Rapid Ca²⁺ fluxes were detected after E. coli 536 infection, consistent with the membrane effects of hemolysin. This was confirmed, using, the hly1/2 double deletion mutant E. coli 536Δhly^{1/2}, which failed to elicit Ca²⁺ fluxes (data not shown). (A-B) CK1α1 activation was inhibited by EGTA (50 μg/ml, 30 minutes) treatment of the cells, supporting the role of Ca²⁺ fluxes. Furthermore, E. coli 536Δhly^{1/2} failed to activate CK1α1, supporting a role of hly. (C) CSNK1A1 expression was up-regulated in cells infected with E. coli 536 supernatant but not ΔPAI I supernatant (qRT-PCR, 4 hours, n=4 experiments).

FIG. 11 shows control experiments addressing the role of hly1 and hly2 for MYC inhibition: (A) c-MYC levels were reduced after infection with E. coli 536ΔHly^{1/2} double deletion mutant or exposure of the cells to bacterial supernatants. (B) Quantification of c-MYC fluorescence in human kidney cells (n=2, compared to uninfected cells). Green=c-MYC; blue=nuclei. Scale bar, 10 μm. (C) Western blot analysis of the corresponding cell extracts. (D-F) Tentative removal of hemolysin from E. coli 536 supernatant by molecular size filtration. The molecular size of hly is 109 kDa, predicting that hly should be excluded from the <100 kDa fraction, which still inhibited c-MYC in human cells, as shown by confocal imaging in (D-E) and Western blots in (F). (G) Analysis of cell toxicity, showing that fractions <100 kDa did not affect cell viability quantified by ATP lite (n.s., compared to untreated cells, n=3 experiments). Mean±S.E.M. *P<0.05, **P<0.01, ***P<0.001, Student's t-test.

FIG. 12 shows the lack of MAX regulation after E. coli 536 infection: Transcriptomic network of MAX regulated genes showing no activation of MAX by E. coli 536 infection, and little regulation of MAX-related genes.

FIG. 13 shows a schematic of the murine MB49 bladder cancer model. On day 0, bladders from C57BL/6 WT mice were emptied and pre-conditioned for 30 minutes by intravesical instillation of 100 μl poly-L-lysine solution (0.1 mg/ml) through a soft polyethylene catheter. Mouse bladder (MB49) tumor cells were instilled (2×10⁵ in 100 μl PBS) on day 0 and allowed to establish for three days, prior intravesical treatment with 100 μl of E. coli 536 supernatant, ΔPAI I supernatant or PBS (sham) on days 3, 5, 7, 9 and 11. Mice were sacrificed before onset of symptoms on day 13 and bladders were collected for further analysis.

FIG. 14 shows the transcriptomic response of tumor-bearing mice treated with E. coli 536 or ΔPAI I supernatant: Whole bladder RNA was subjected to transcriptomic analysis. (A) Heat map of regulated genes in tumor bearing mice treated with E. coli 536 supernatant, ΔPAI I supernatant or sham treated controls compared to healthy bladders. The results show a reduction in the number of regulated genes in the E. coli 536 group (FC>2.0). (C) Heat map of cancer related canonical pathways identified in tumor bearing mice. Therapeutic effect of E. coli 536 supernatant compared to sham treated mice. The ΔPAI I supernatant did not significantly alter gene expression compared to sham treated mice. (C) Scatter plots of signal intensity of probe sets in tumor bearing mice treated with E. coli 536 supernatant, ΔPAI I supernatant or PBS (sham) and compared to healthy bladders. The results show that E. coli 536 supernatant suggest a loss of genes that are regulated in the sham group and an increased similarity to the healthy controls. This effect was not seen in mice treated with ΔPAI I supernatant. The results indicate that there is less activation of cancer related pathways in the E. coli 536 supernatant treated mice.

FIG. 15 shows c-MYC suppression by uropathogenic E. coli strains.

c-MYC protein levels in A498 cells infected with uropathogenic strains E. coli CFT073 or E. coli 536 or asymptomatic bacteriuria (ABU) strain E. coli 83972 (10⁵ CFU/ml, 1 or 4 hours), relative to PBS control. a, Confocal images of infected cells (Scale bars=10 μm). b, Quantification of c-MYC levels based on fluorescence intensity from (a), n=3 experiments. c, Representative Western blot (WB) showing a reduction in c-MYC levels in extracts from infected cells, n=3 experiments. d, c-MYC levels in cells infected with acute pyelonephritis (APN) (n=36) or ABU (n=20) isolates, means of 2 experiments per strain. Quantification of c-MYC levels by WB of infected cell extracts, 4 hours). Histogram inset shows group means. For cell viability data, see FIG. 21. e, Schematic of study protocol, febrile UTI in infancy. f, Reduction in MYC expression during acute infection compared to the six-month follow up, group-wise analysis (qRT-PCR). g, Intra-individual analysis of MYC mRNA levels during acute infection. h, Gene set enrichment analysis (GSEA) confirming the inhibition of MYC target genes during acute infection. IL-6-JAK and STAT3 signalling was upregulated, consistent with the known acute inflammatory response in this patient group. Data is presented as means±SEMs and analysed by two-tailed t-test (b), Mann-Whitney U-test (d), or by Wilcoxon matched pairs signed rank test (f), *P<0.05, **P<0.01, ***P<0.001.

FIG. 16 shows a systematic gene deletion strategy identifying bacterial effectors of c-MYC inhibition.

a, Schematic representation of the E. coli 536 chromosome, indicating the positions of pathogenicity islands (PAIs) I to V. b-c, E. coli 536 deletion mutants (PAI I₅₃₆ to PAI V₅₃₆ and PAI I-II₅₃₆) were screened for effects on cellular c-MYC levels in A498 cells (10⁵ cfu/ml, 4 hours) using Confocal microscopy (b) and WB analysis (c) of infected cell extracts. PAI I₅₃₆ was identified as a loss-of-function mutant (Scale bars=10 μm. See also FIG. 23). d, 536Δhly I (on PAI I₅₃₆) and 536Δhly II (on PAI II₅₃₆) showed partial effects on c-MYC expression. e, Strategy used to identify genes on PAI I₅₃₆ affecting cellular c-MYC levels. f, Loss-of-function phenotype in PAI IΔ2 deletion mutants and g, the PAI IΔ2-5.3 sub-clone. WB analysis of infected cell extracts. The deleted gene cluster encodes a metal-ion ABC transporter. h, A deletion of the corresponding ABC transporter in E. coli CFT073 reproduced the phenotype of the PAI IΔ2-5.3 sub-clone (WB analysis). i, Screening of metal ions for effects on c-MYC degradation, suggesting that Zn²⁺ fluxes regulated by the bacterial ABC transporter affect the production of the bacterial MYC inhibitor. j, Stimulation of A498 cells with E. coli 536 supernatant reproduced the effects of infection suggesting that the bacterial effector is secreted. The PAI I₅₃₆ supernatant served as a negative control (WB analysis of whole-cell extracts). k, Schematic predicting that Zn²⁺ accessibility may regulate the expression and release of c-MYC inhibitor(s) by bacteria.

FIG. 17 shows mechanisms of accelerated c-MYC degradation by bacterial effector molecules.

a, Table of proteins detected in the supernatant of E. coli 536 but not in PAI I₅₃₆. Hly and the Lon protease were selected for further study (LC-MS/MS, see also FIG. 24, 25). b, Kinetics of c-MYC degradation by recombinant Lon protease in vitro (n=4 experiments). c, Schematic of the c-MYC domain structure and fragments degraded by Lon protease in these domains (identified by LC-MS/MS). Domains predicted to be degraded are indicated by dashed lines. d-e, Co-immunoprecipitation by GST-tagged c-MYC protein of (d), recombinant Lon protease e, Lon protease from the E. coli 536 and 536Δhly^{1/2} supernatants (WB analysis). A 536Δlon supernatant was used as a negative control. f, Dose-dependent uptake of recombinant Lon protein (red) by human kidney cells and a concomitant reduction in c-MYC staining (green). Confocal microscopy, 4 hours incubation. Quantification of data in f, (n=3). g-h, c-MYC protein levels in cells infected with E. coli 536, 536Δhly^{1/2}, PAI I₅₃₆ or 536Δlon (4 hours). g, Confocal imaging and h, WB analysis. Scale bars=10 μm. Data is presented as means±SEMs and analysed by paired t-test. *P<0.05, **P<0.01, ***P<0.001. i, Comparative analysis of protein degradation by recombinant Lon. c-MYC was degraded after 3 hours, unlike MYC related proteins (MAX, CEBPD), upstream regulators of MYC (AKT, CDK9, CPEB1) and housekeeping proteins (GRP75, actin, Histone H3).

FIG. 18 shows that Transcriptional regulators of c-MYC expression are inhibited by infection.

a, Gene expression analysis identifies of A498 cells infected with E. coli 536 or PAI I₅₃₆. Inversely regulated genes are identified. Red: upregulated, blue: down-regulated, black: not significantly regulated (cut-off fold change ≥1.41). b, A subset of MYB-related genes was inversely regulated. c, Confocal imaging of c-MYB protein levels in infected cells and quantification compared to cells exposed to the bacterial supernatant. Scale bar, 10 μm. d, MYB mRNA levels in cells exposed to E. coli 536 bacteria or supernatant, compared to PAI I₅₃₆. (n=4). e, Confocal imaging of CEBPD protein levels in infected cells and quantification compared to cells exposed to the bacterial supernatant. Scale bar, 10 μm. f, CEBPD mRNA levels in cells exposed to E. coli 536 bacteria or supernatant, compared to PAI I₅₃₆. (n=4). g, MYC and MAX expression levels in kidney epithelial cells infected with E. coli 536 or PAI I₅₃₆ (qRT-PCR, n=4 experiments). h, MAX protein staining in infected cells (confocal microscopy). i, Quantification of fluorescence intensity in h, (n=2). j, MYB and CEBPD expression in patients with acute pyelonephritis, compared to paired follow-up samples. MYB levels were reduced during acute infection. k, Model of c-MYC regulation by bacteria. Three mechanisms of c-MYC degradation have been uncovered. (i) Degradation by the bacterial Lon protease. (ii) Activation of CK1α1, by α-hemolysin-dependent Ca²⁺ fluxes, targeting c-MYC for proteasomal degradation. (iii) c-MYB degradation by CK1α1 activation and transcriptional repression of CEPBD, suggesting mechanisms, which may lead to inhibited c-MYC expression. Data is presented as means±SEMs and analysed by paired t-test (c-i) or Wilcoxon matched pairs signed rank test (j). *P<0.05, **P<0.01.

FIG. 19 shows c-MYC inhibition by the Lon protease in cancer cells and therapeutic efficacy against bladder cancer.

a, c-MYC inhibition by E. coli 536 infection in Burkitt lymphoma cells (Daudi and Raji) and human or mouse bladder- (HTB-9 and MB49), lung- (A549), gastric- (AGS), colon- (DLD1) and hepatic (HEP-G2) carcinoma cells. b, MYC inhibition by E. coli 536 supernatants in HTB9, A549, DLD1 and MB49 cells but not by PAI I₅₃₆, 536Δhly^{1/2} or 536Δlon supernatants (See also FIG. 30) or RPMI. WB of cell extracts (n=3 experiments per cell type). c, Schematic of the protocol used to establish and treat MB49 bladder cancer in C57BL/6J WT mice. d, Photomicrographs of bladders from the sham group compared to mice treated with the Lon protease or E. coli 536 supernatants. e, Quantification of bladder weight and bladder size in mice treated with the Lon protease or E. coli 536 supernatants compared to the sham group. Data for individual mice is presented (n=13 mice per group). Medians are indicated by lines. Data is analysed by the Kruskal-Wallis test. f, Whole bladder frozen tissue sections stained with H&E, representative images are shown. The tumor mass is indicated by the dotted lines. g, c-MYC protein staining in mice treated with the E. coli 536 supernatant or Lon protease compared to the sham group. Confocal microscopy, the bladder lumen is indicated by a dotted line, one representative images are shown from n=5 mice per group. h, Heatmap of significantly regulated genes compared to tumor free controls in Lon treated mice versus the sham group (n=2 mice per group). i, Cancer related genes were inhibited in Lon treated mice compared to the sham group, including a subset of bladder cancer-related genes. j, Functional annotation of regulated cancer gene categories. Histogram of cancer biofunctions regulated in Lon treated mice compared to the sham group. Blue=z-score, orange=−log P-value. k, Representation of c-MYC-related gene network inhibited in Lon treated mice compared to the sham group. Samples for gene expression analysis were randomly selected. Red: upregulated genes, blue: down-regulated genes (cut-off fold change ≥2, P<0.05).

FIG. 20. shows the therapeutic efficacy of Lon in a colon cancer model a, Schematic of the treatment protocol used in Apc^{Min/+} mice, which develop intestinal polyps that progress to colon cancer. Apc^{Min/+} mice were treated by oral gavage (200 μl) with recombinant Lon Protease (250 μg/ml), twice daily for 14 days. b, Dissection photomicrographs of representative intestinal segments, illustrating the reduction in polyp number (arrowheads indicate polyps). c, Methylene blue stained whole mounts of intestinal segments. d, Quantification of total polyp number in Lon treated mice compared to the sham group (n=10 mice per group) or mice sacrificed prior to the onset of treatment (n=8). e, Functional annotation of regulated gene categories revealed a predominance of cancer related genes in Lon treated mice compared to the sham group, including a subset of colon cancer-related genes. f, Heat map of significantly regulated genes in Lon treated mice or the sham group, compared to healthy C57BL/6J WT mice (n=3 Lon, 2 sham and 2 healthy). g, Histogram of regulated cancer functions in Lon treated mice compared to the sham group. h, MYC-related network in Lon treated mice compared to the sham group. i, Kaplan Mayer plot showing increased survival in Lon treated mice (n=6) compared to the sham group (n=7). j, Reduction in total intestinal polyp number and k, body weight loss in mice receiving Lon treatment compared to the sham group. I, Heat-map of regulated genes and m, volcano plot of significantly regulated probes in Lon treated mice at long term-follow up compared to the sham group (n=5 Lon, 7 Sham). Samples for gene expression analysis were randomly selected. Red: upregulated genes, blue: down-regulated genes (cut-off fold change ≥2, P<0.05). Data for individual mice is presented, with lines representing the medians. Kruskal-Wallis test (d) or Mann-Whitney U-test (j, k).

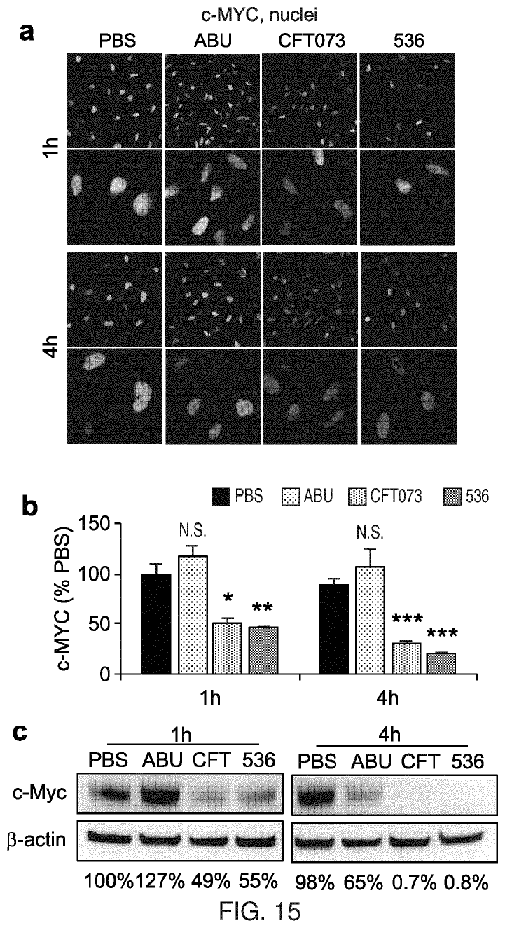

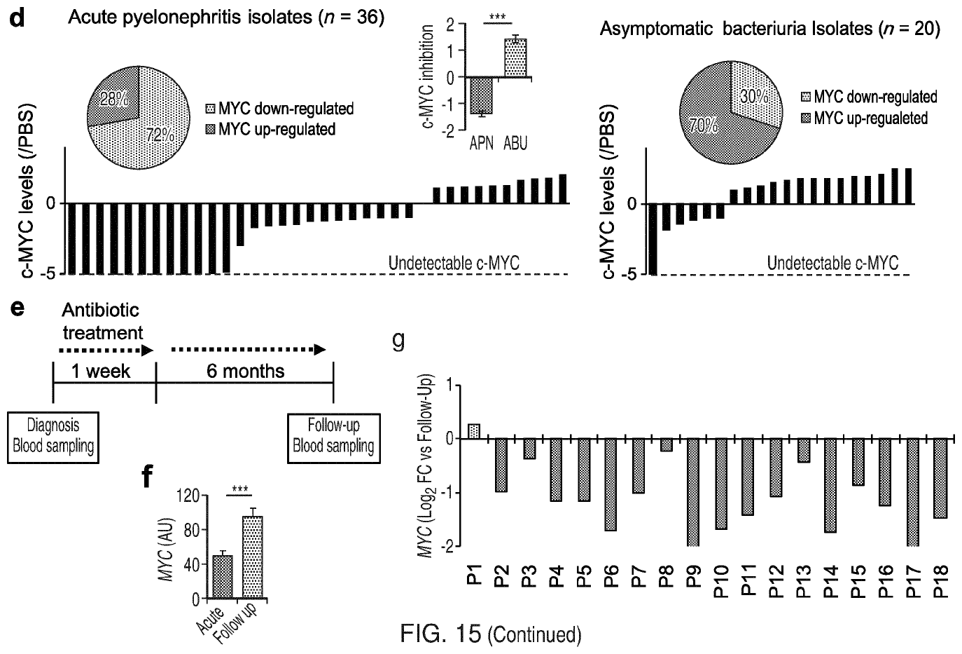

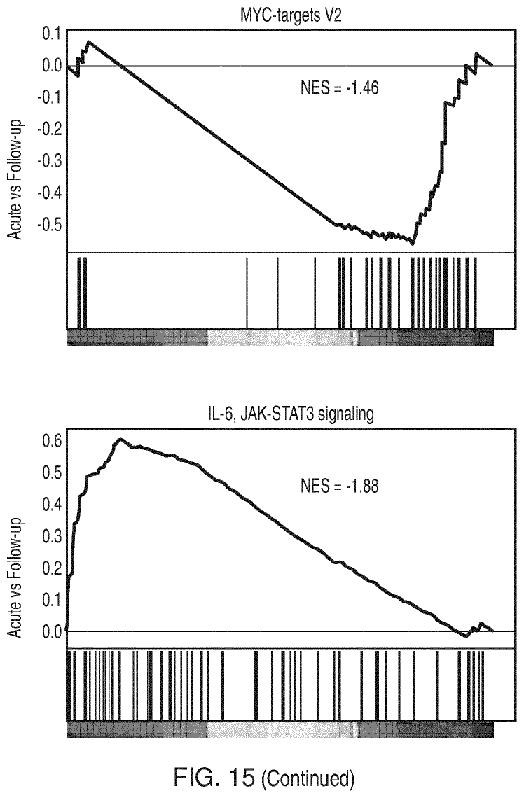

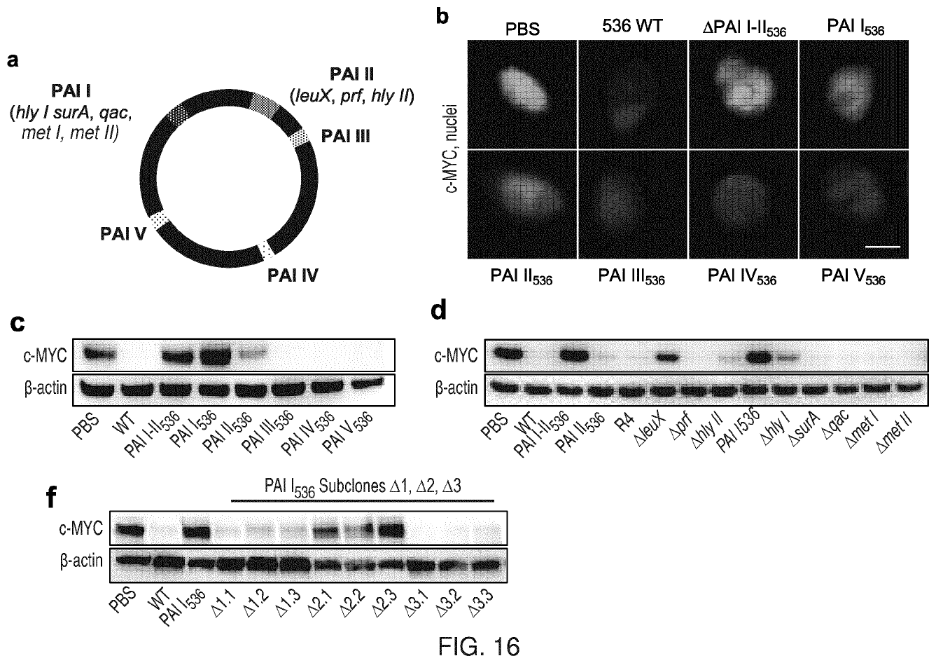

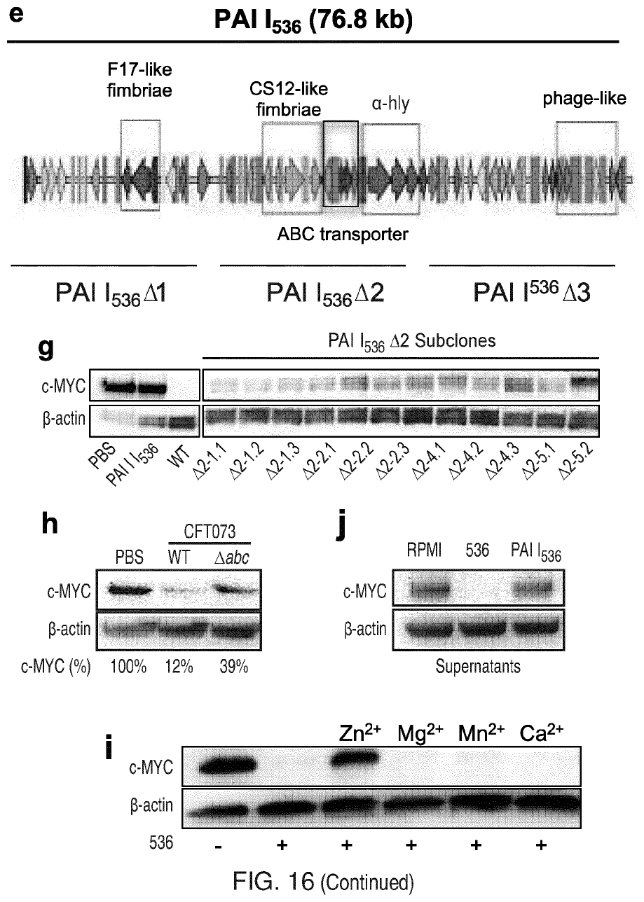

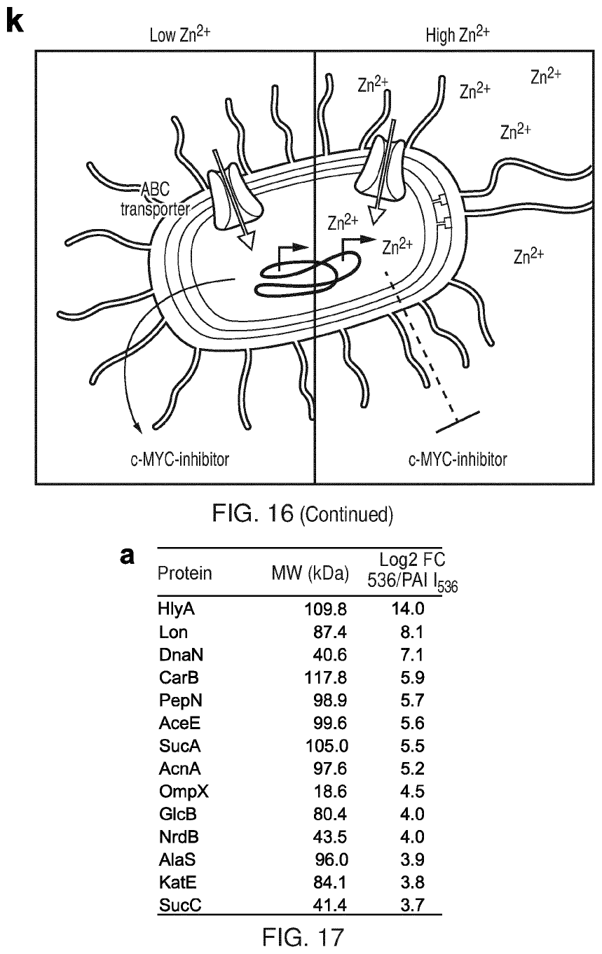

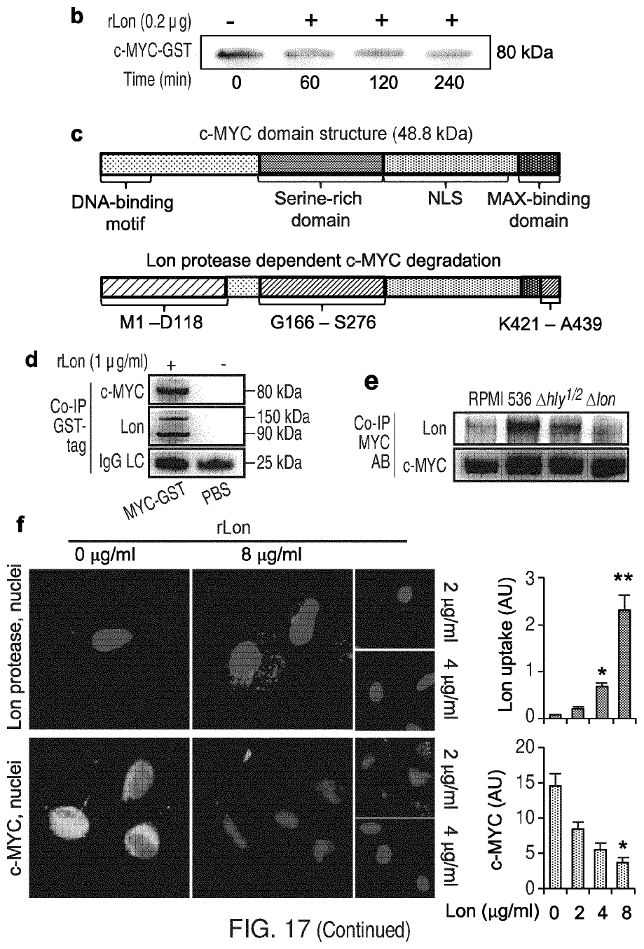

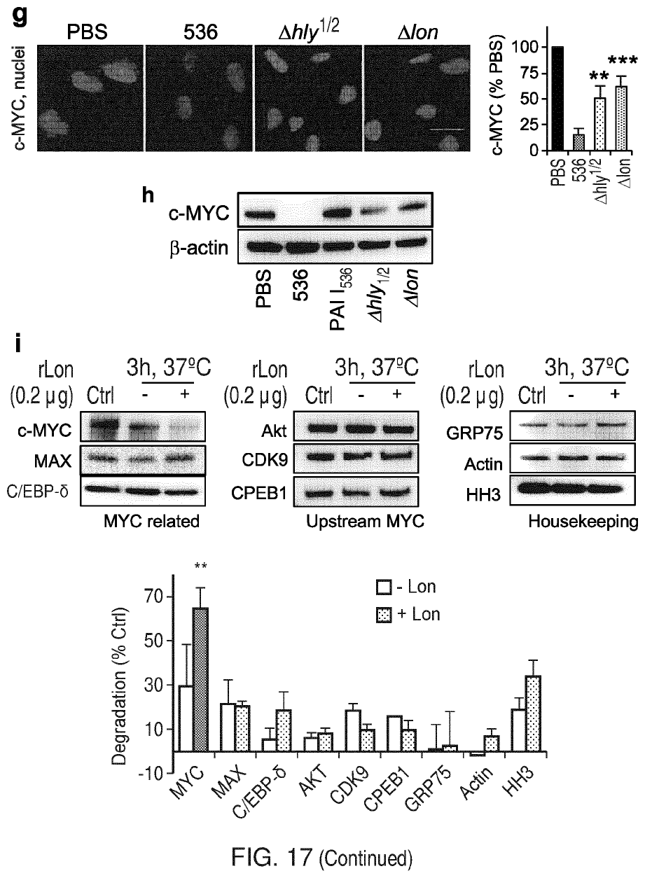

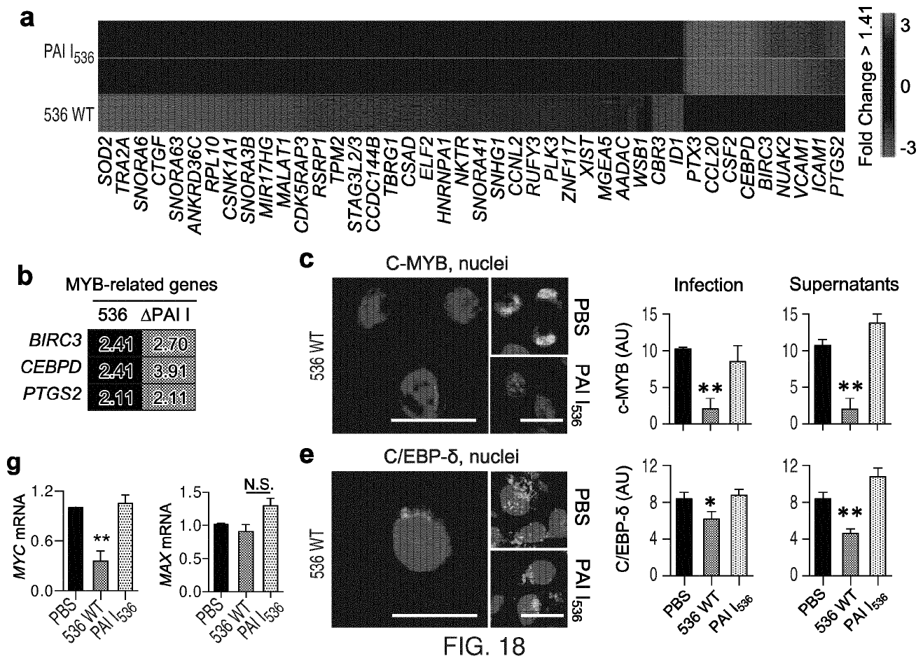

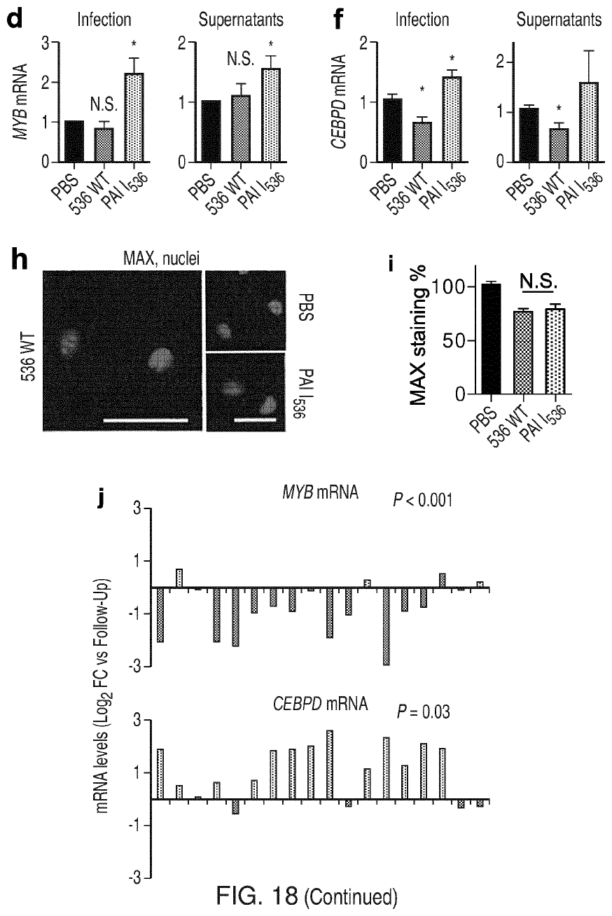

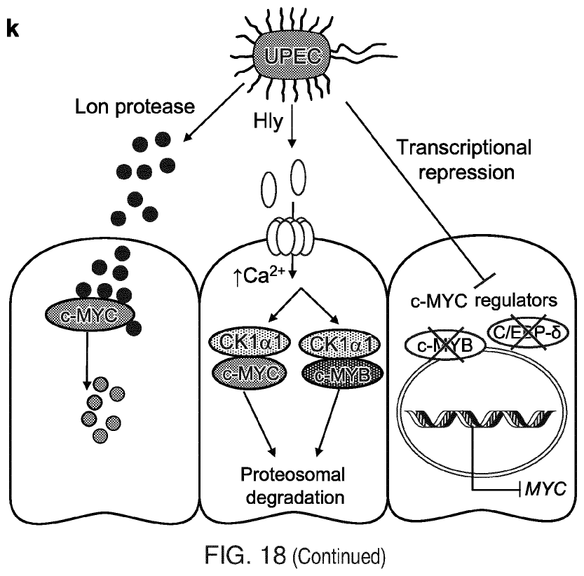

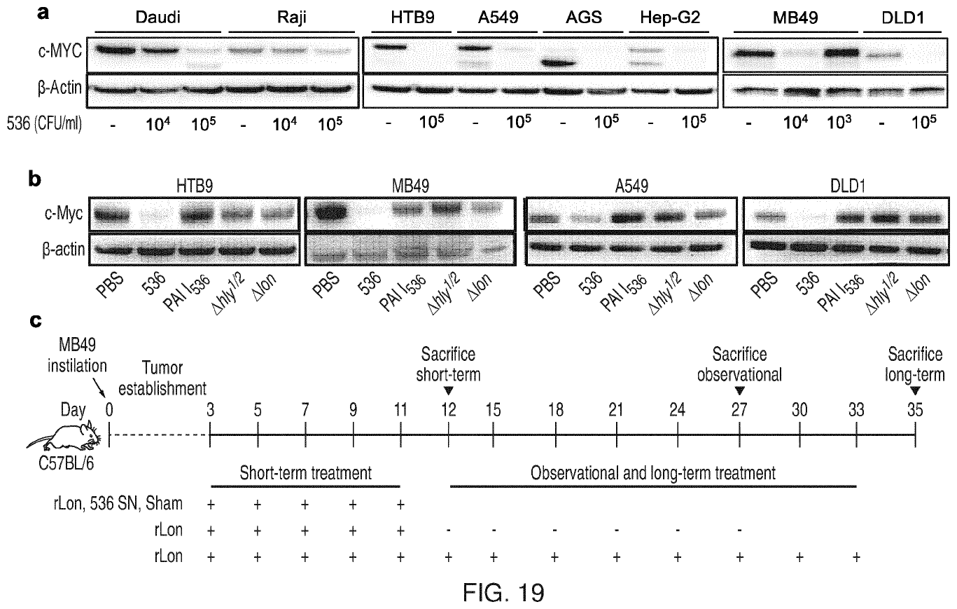

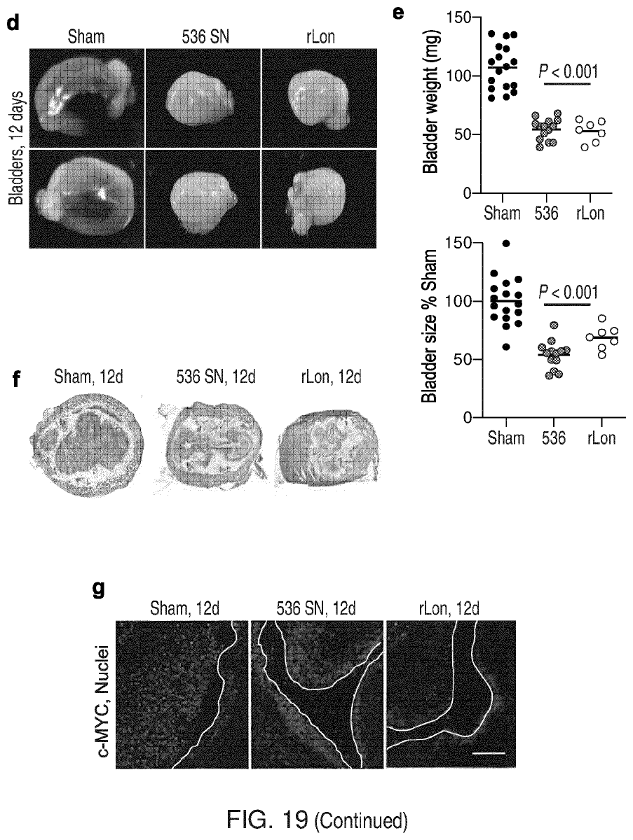

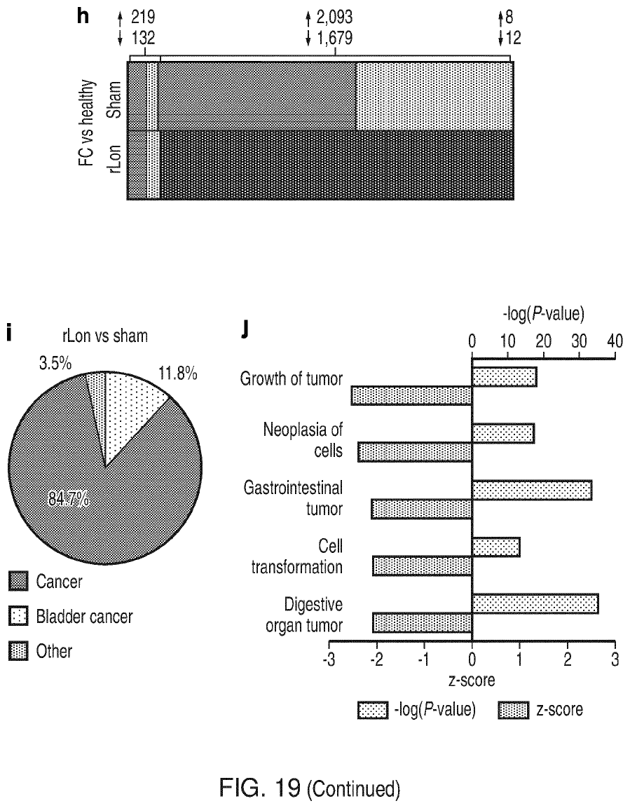

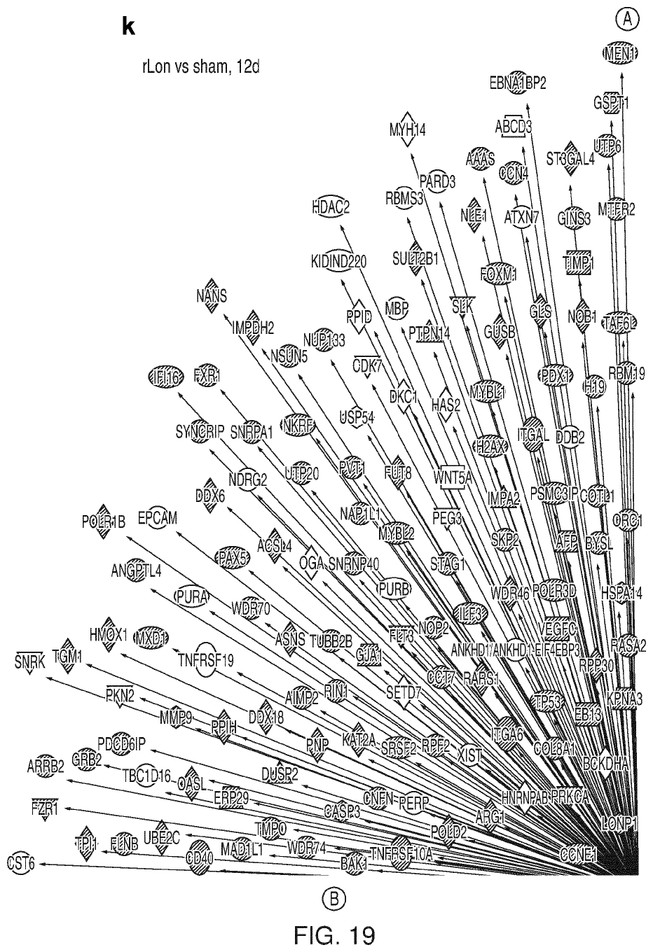

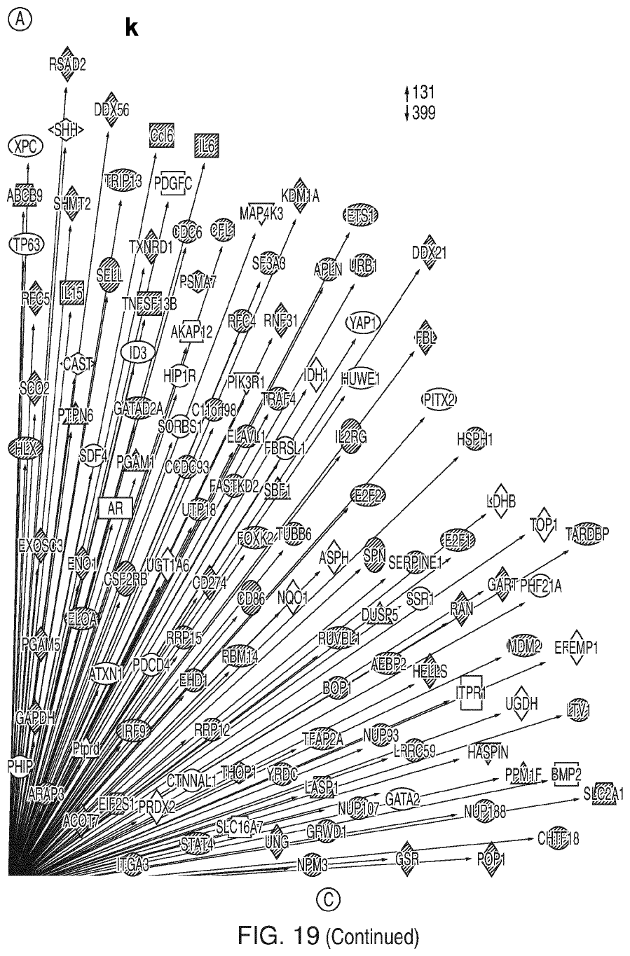

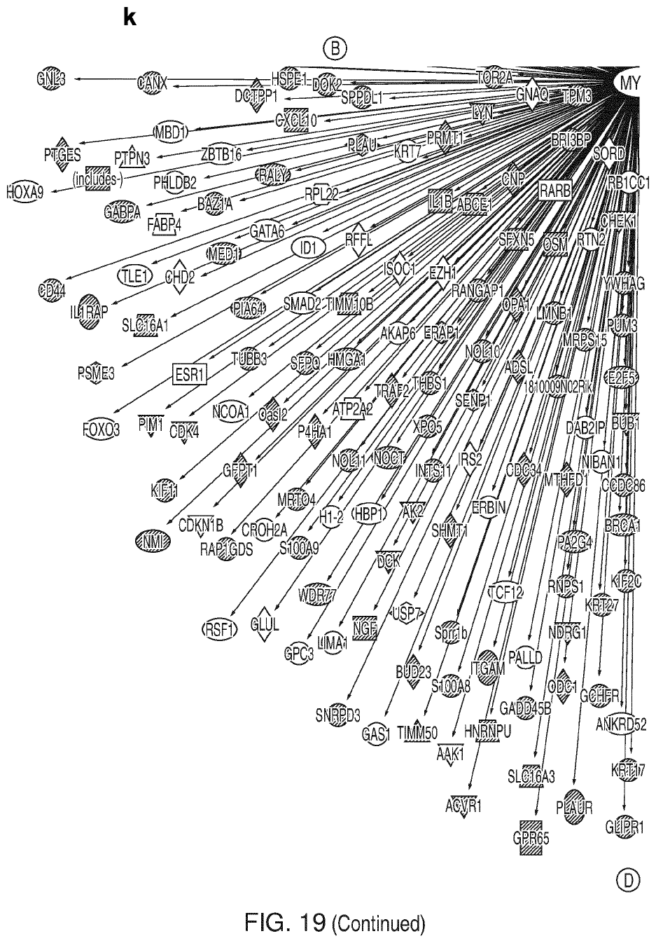

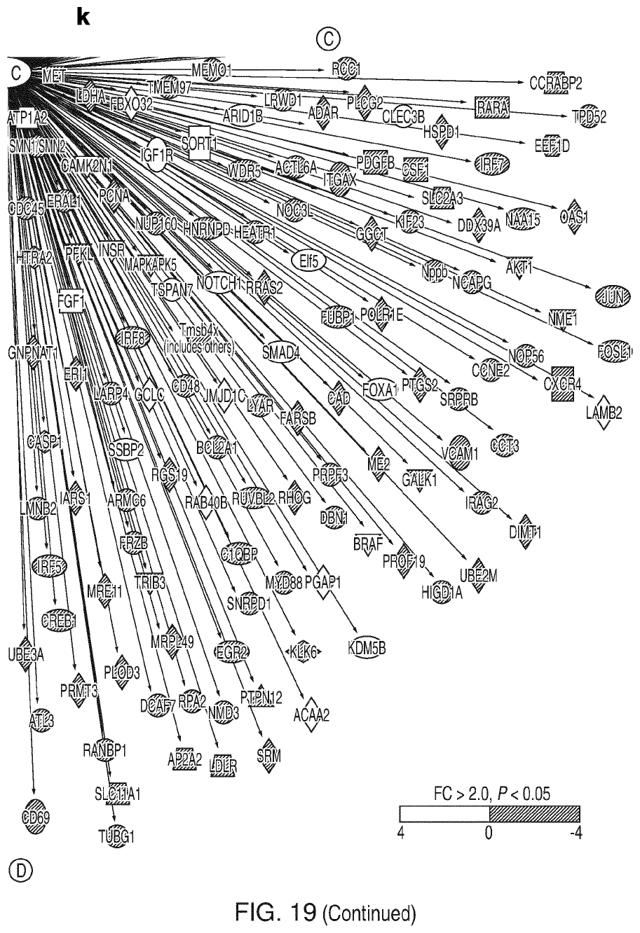

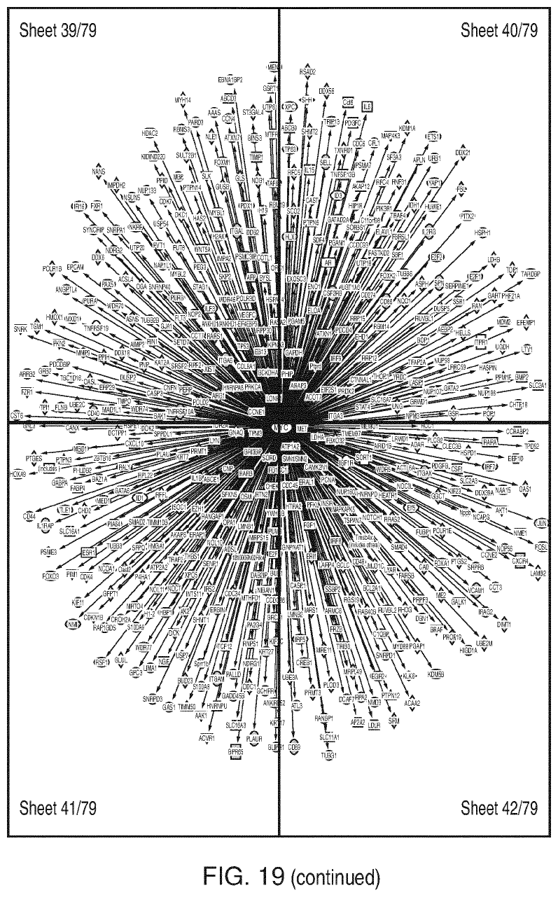

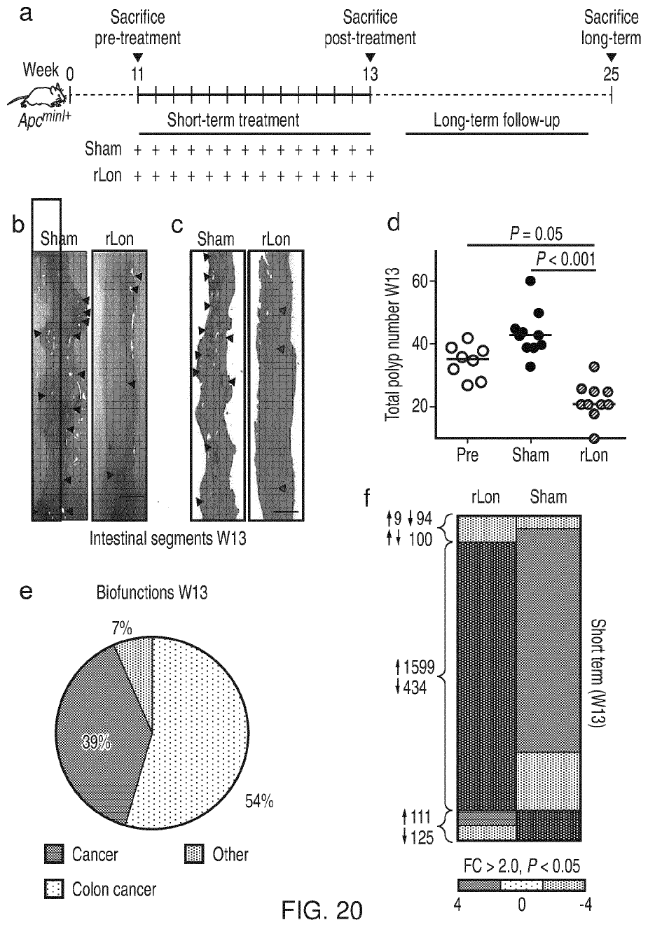

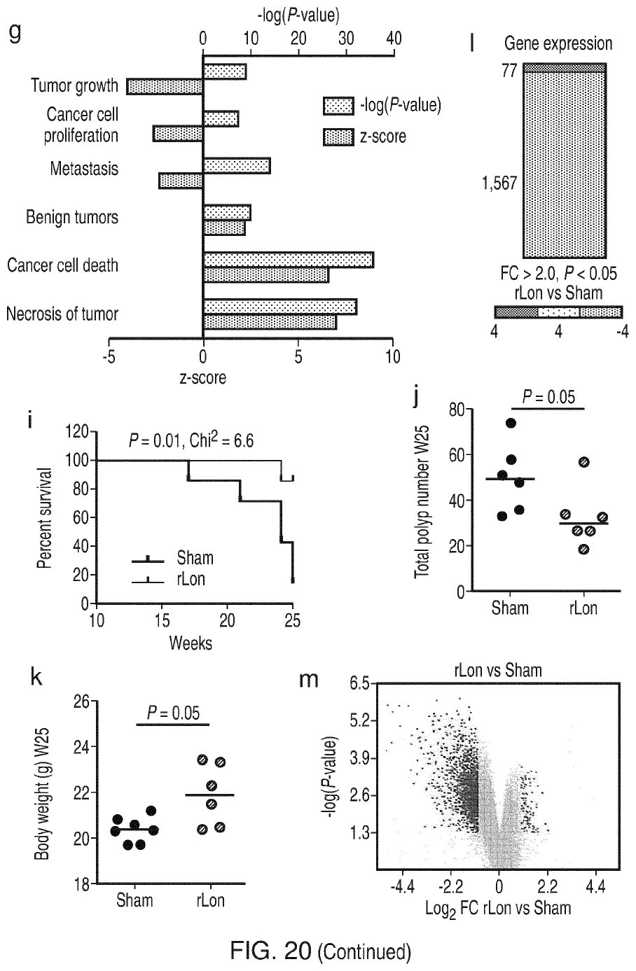

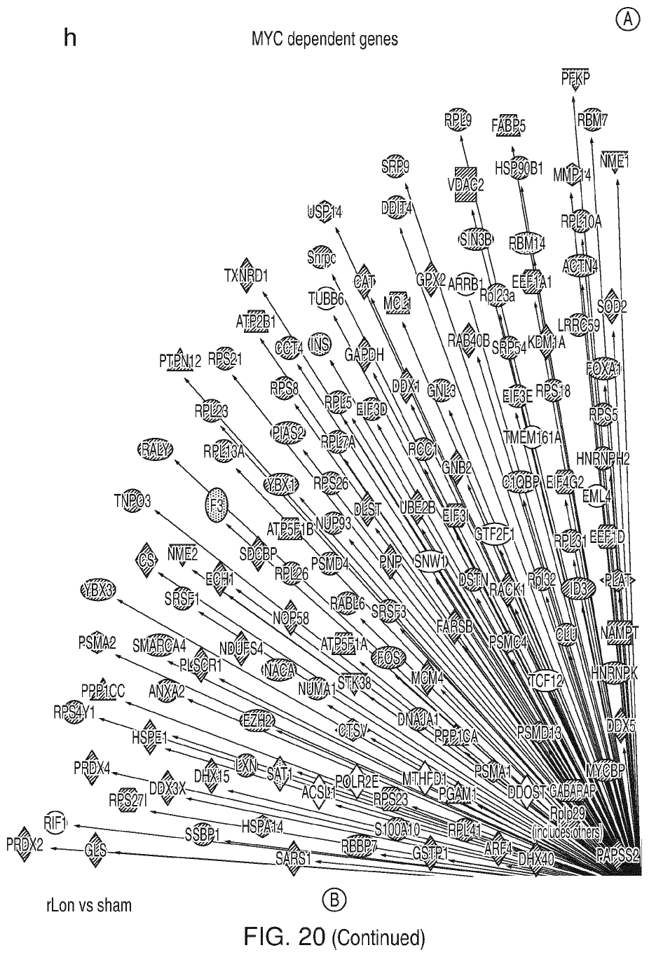

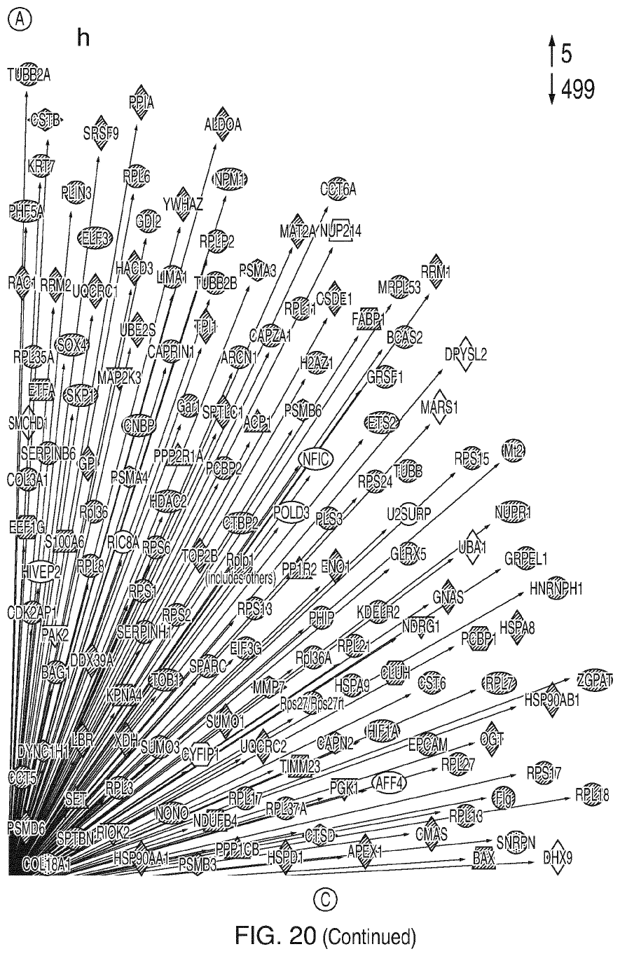

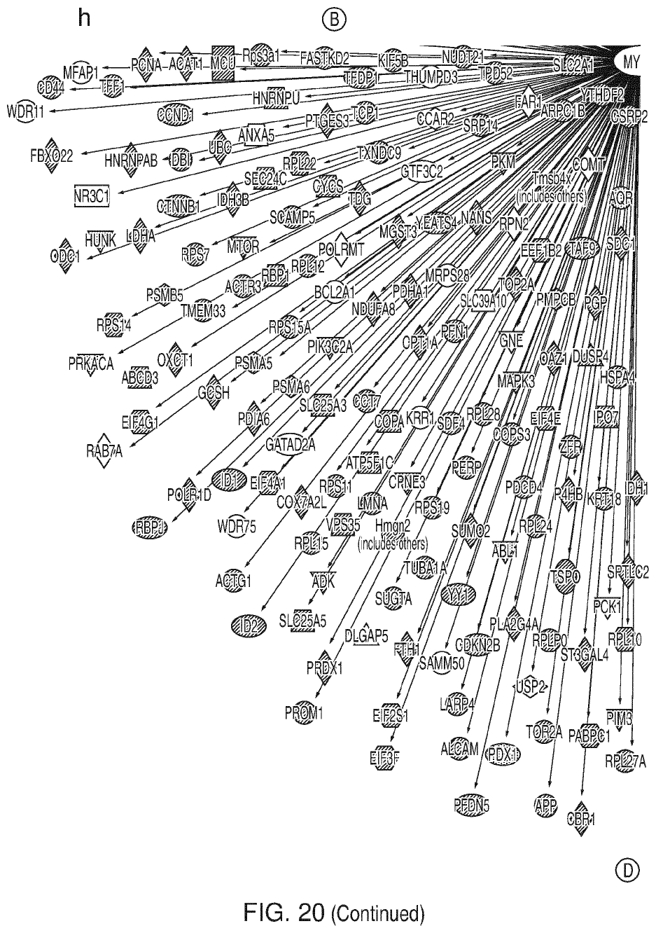

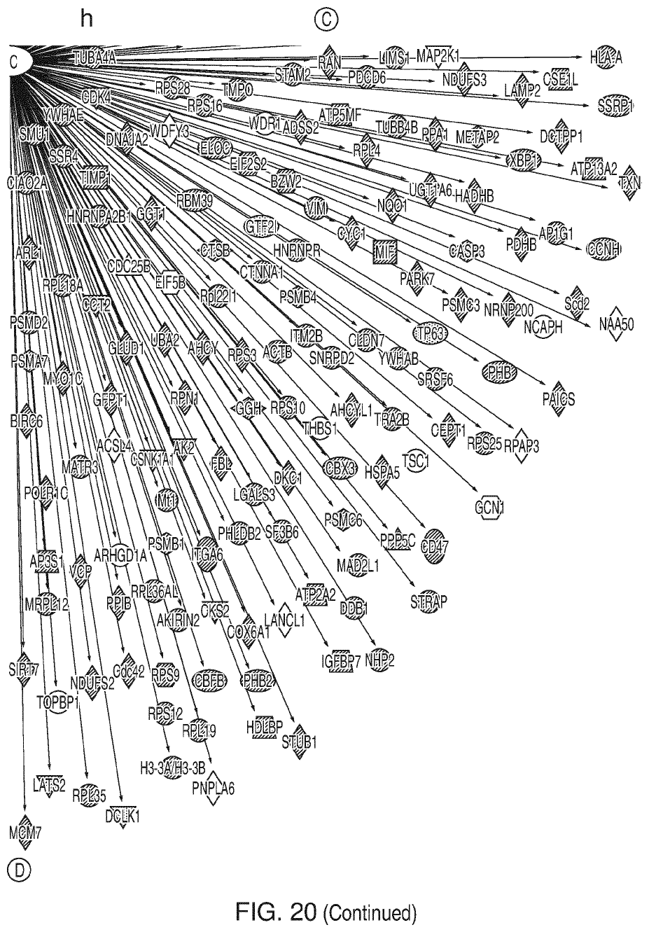

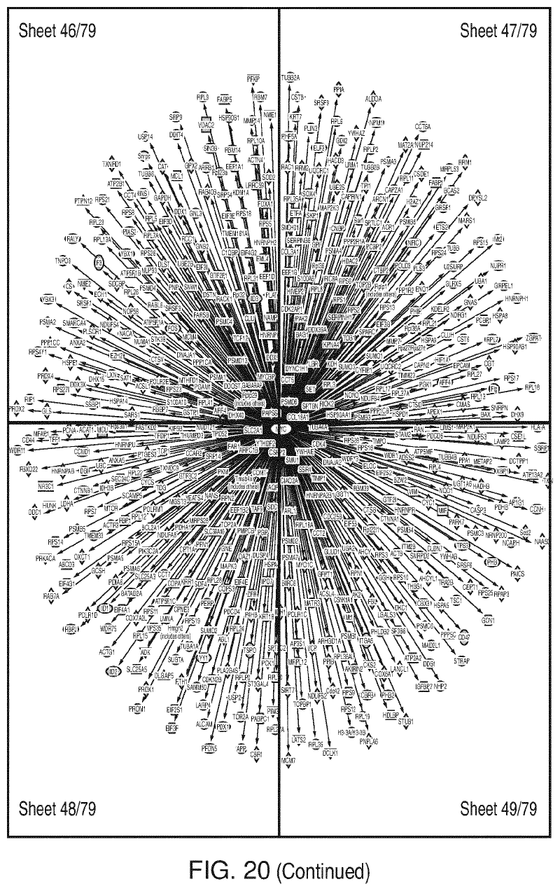

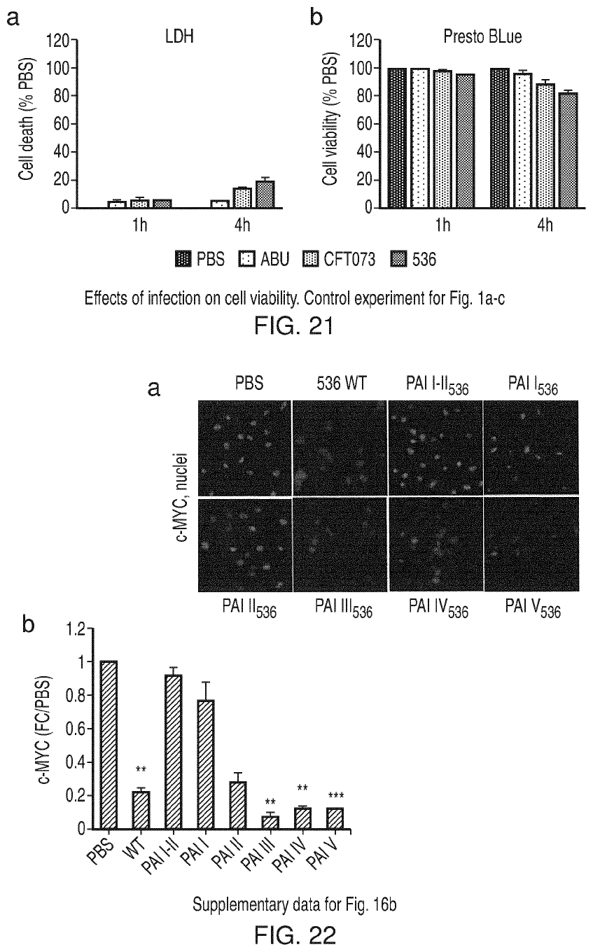

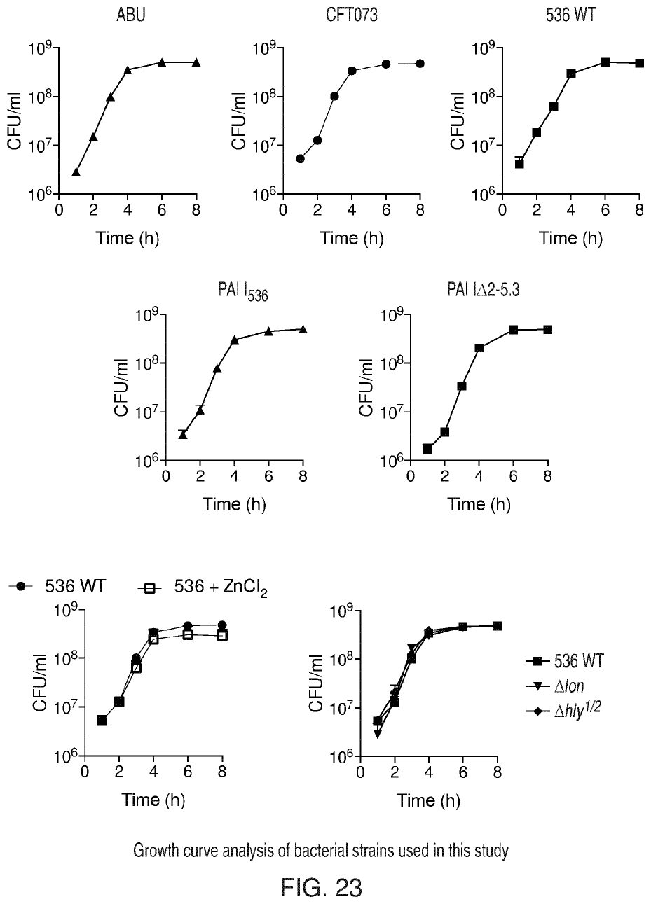

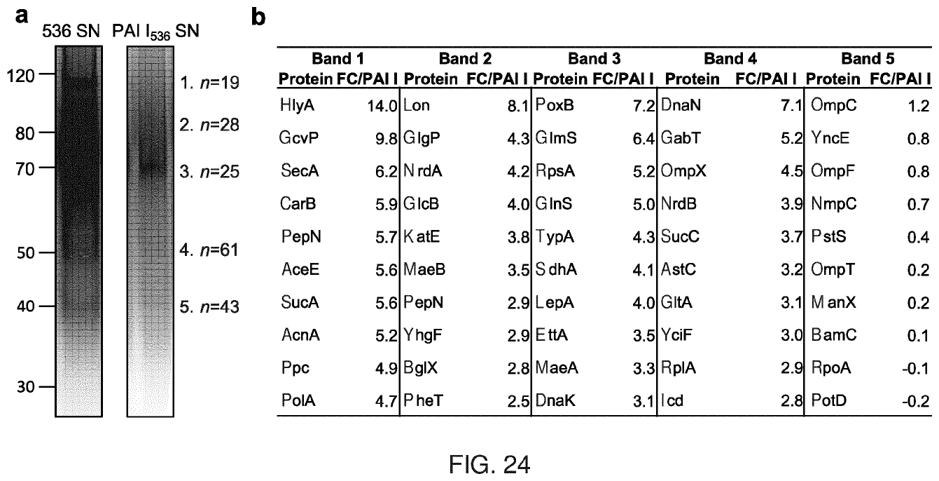

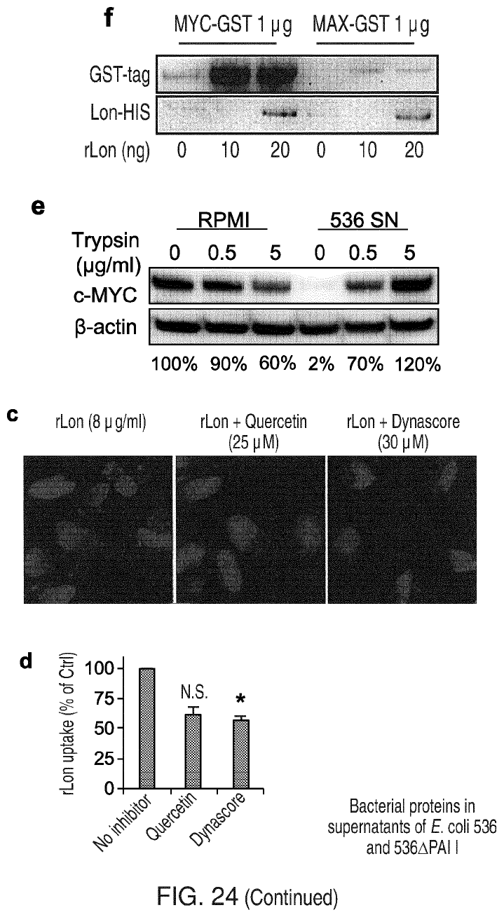

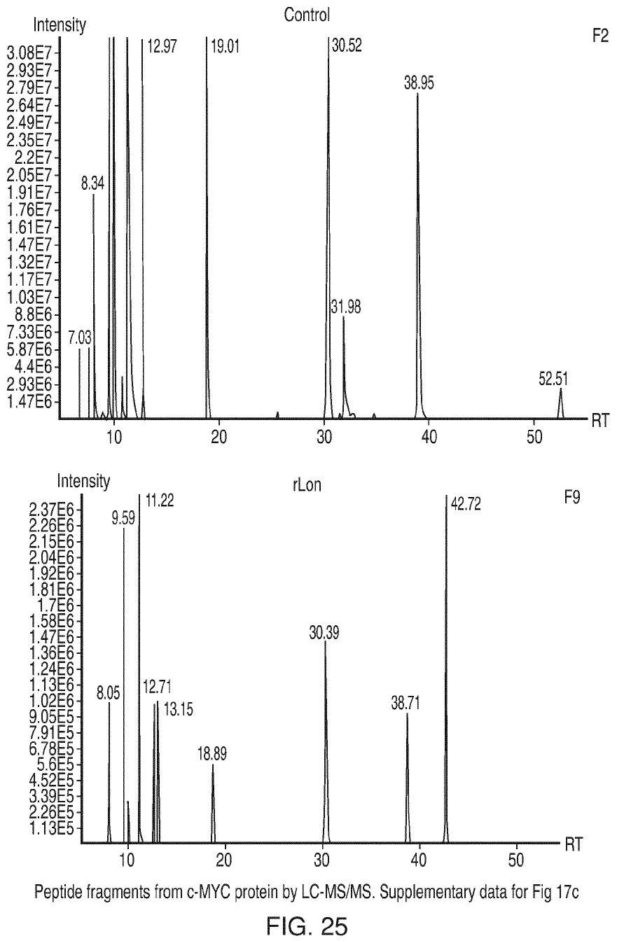

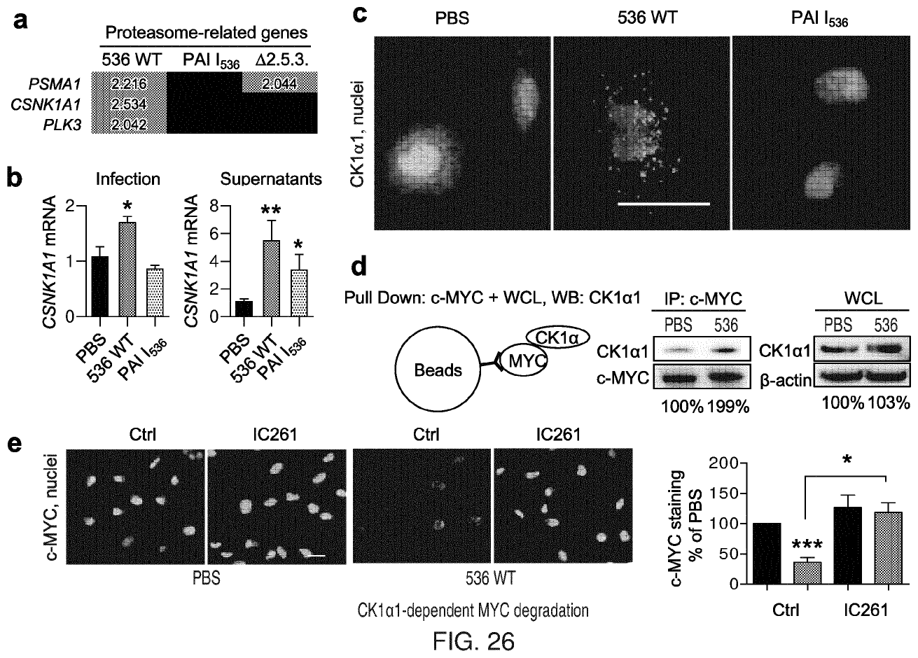

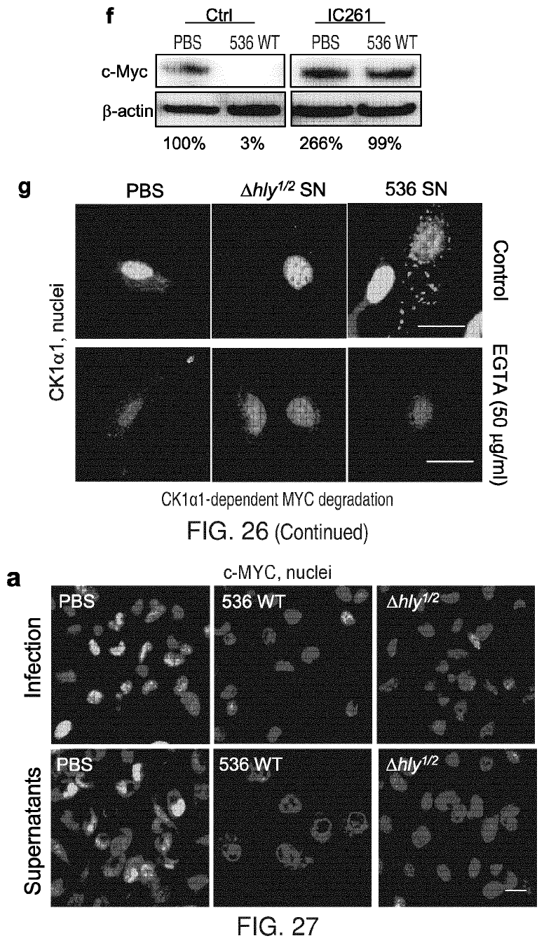

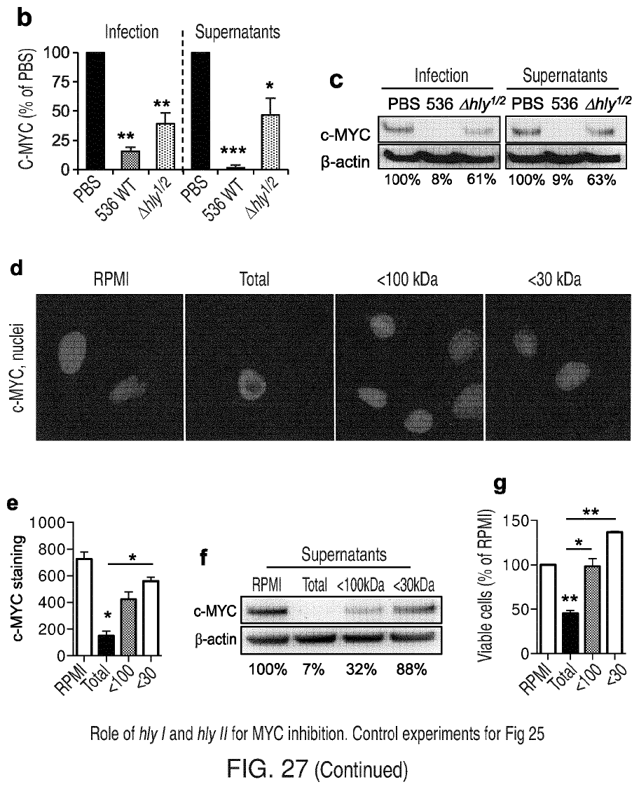

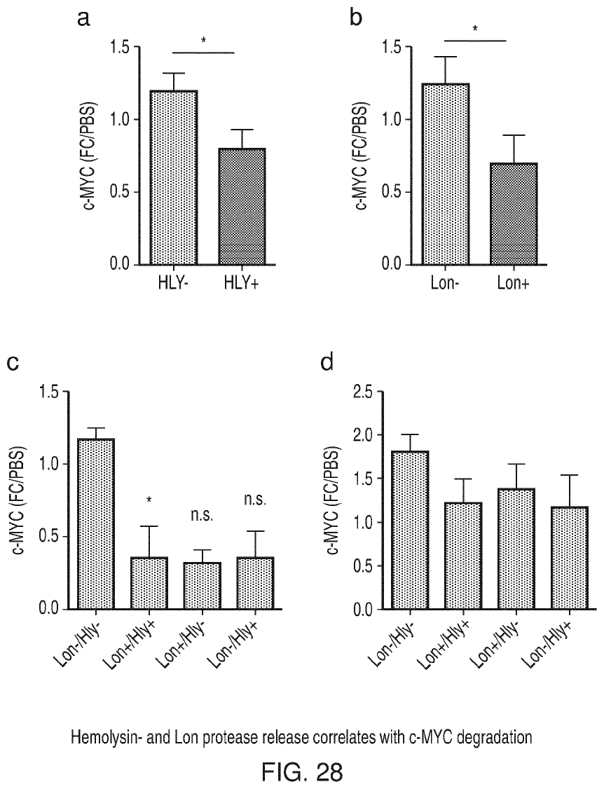

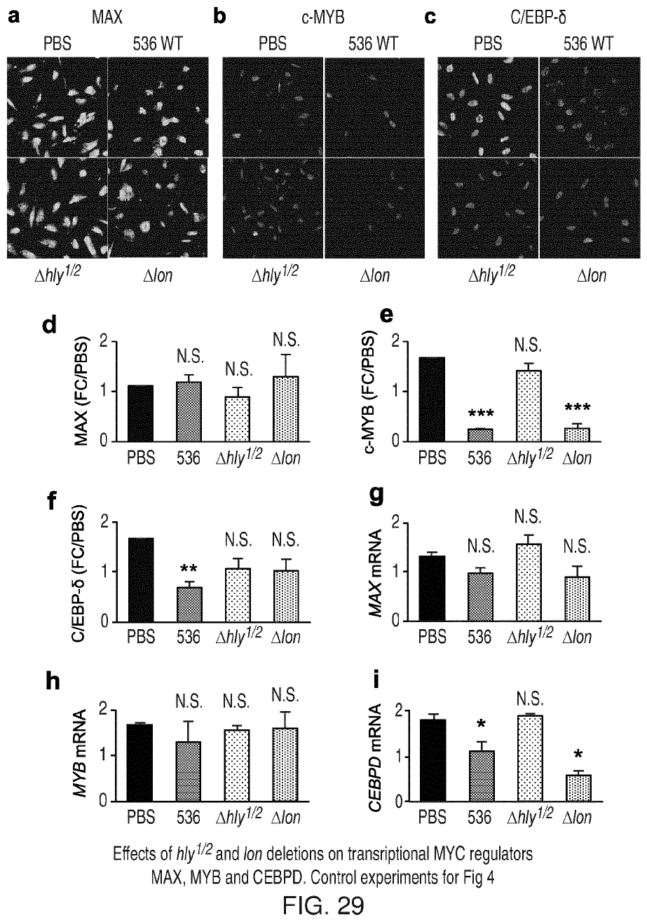

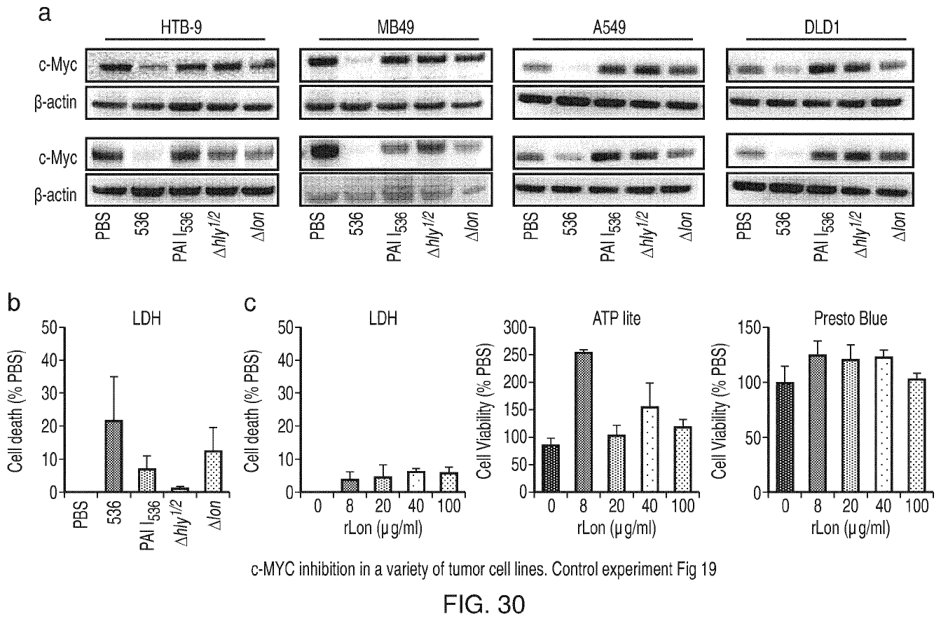

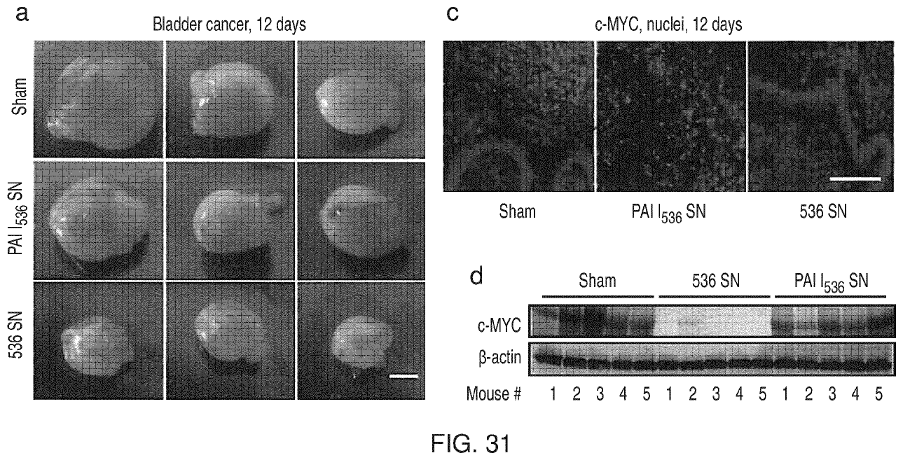

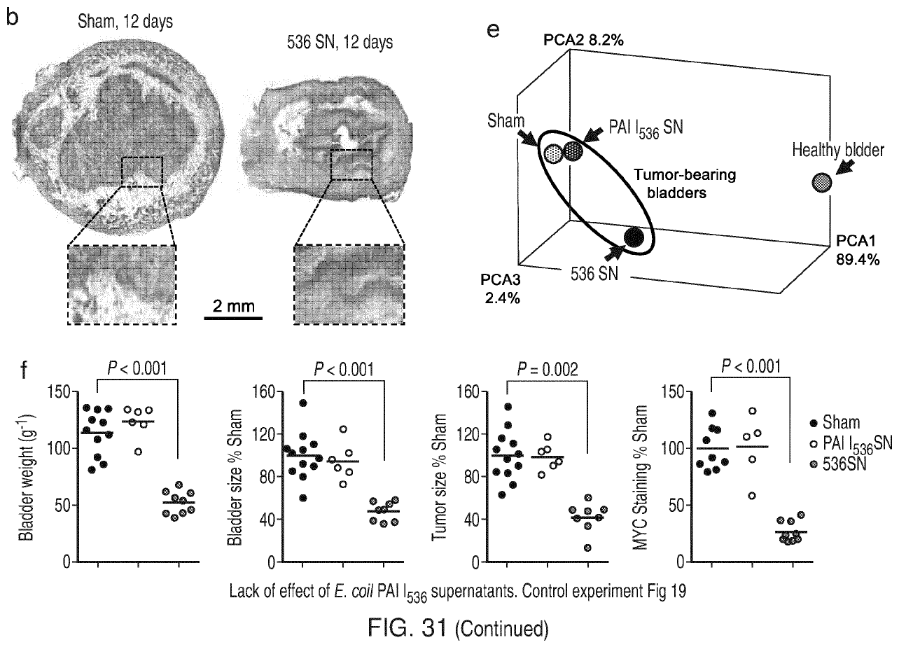

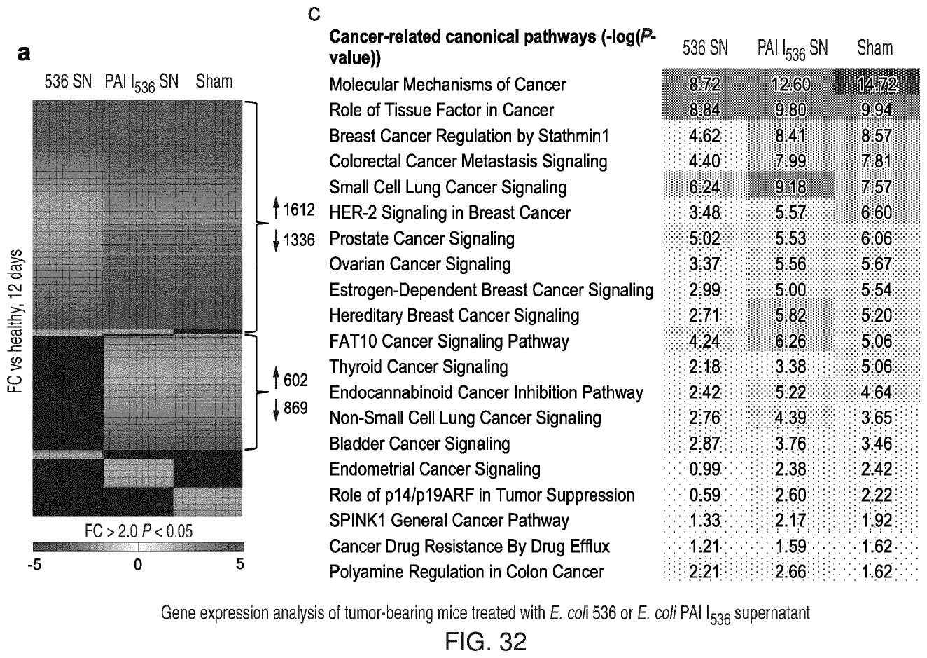

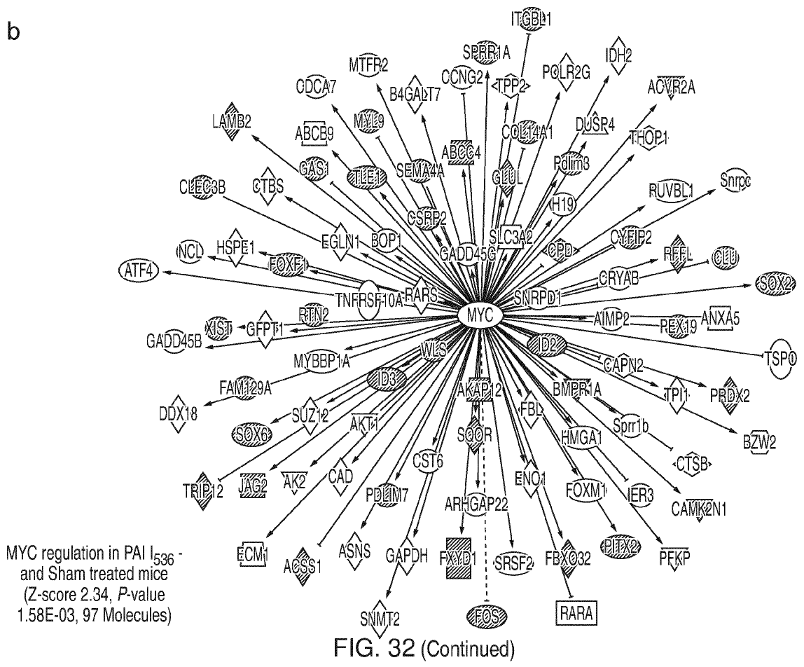

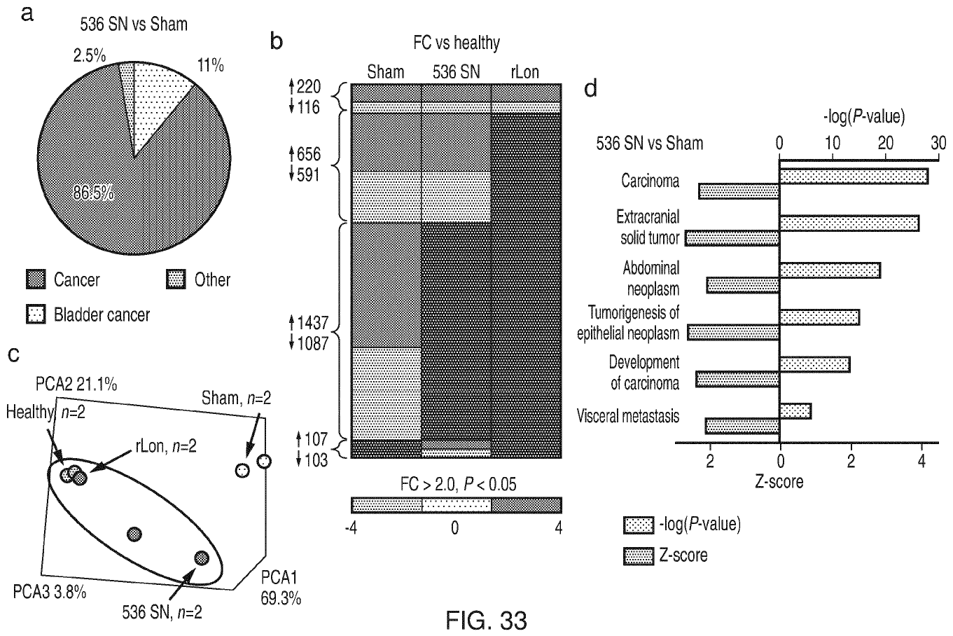

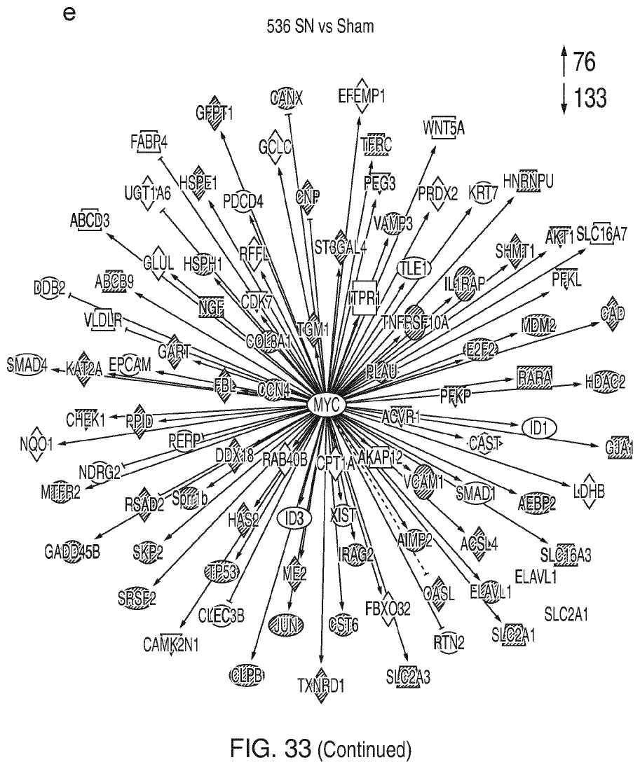

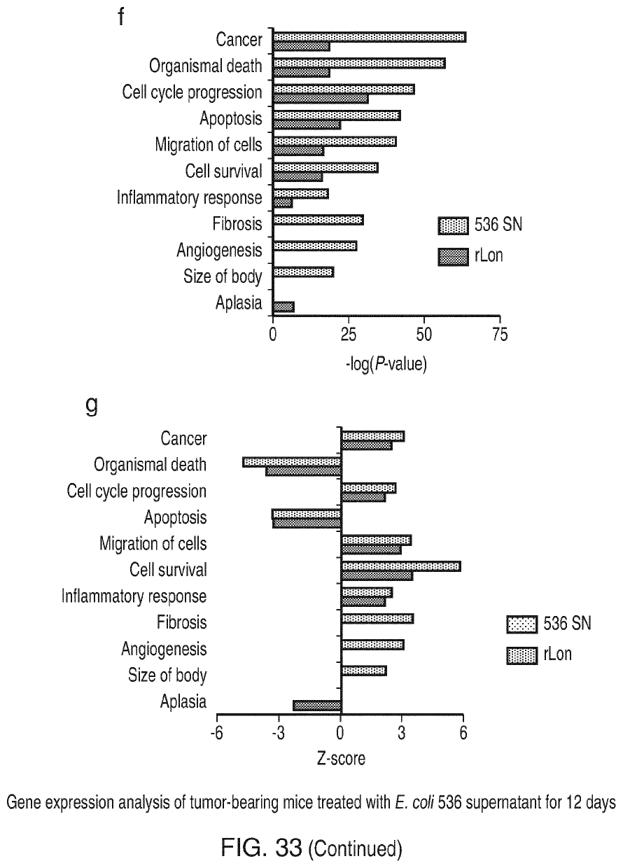

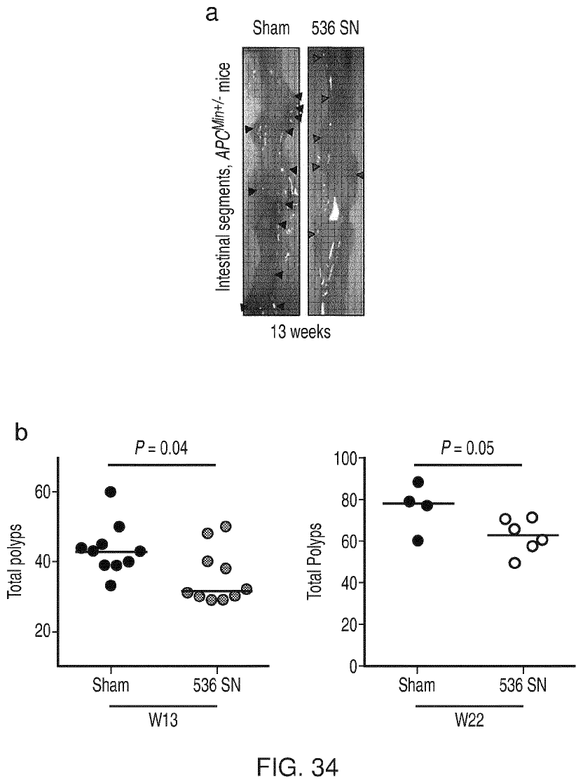

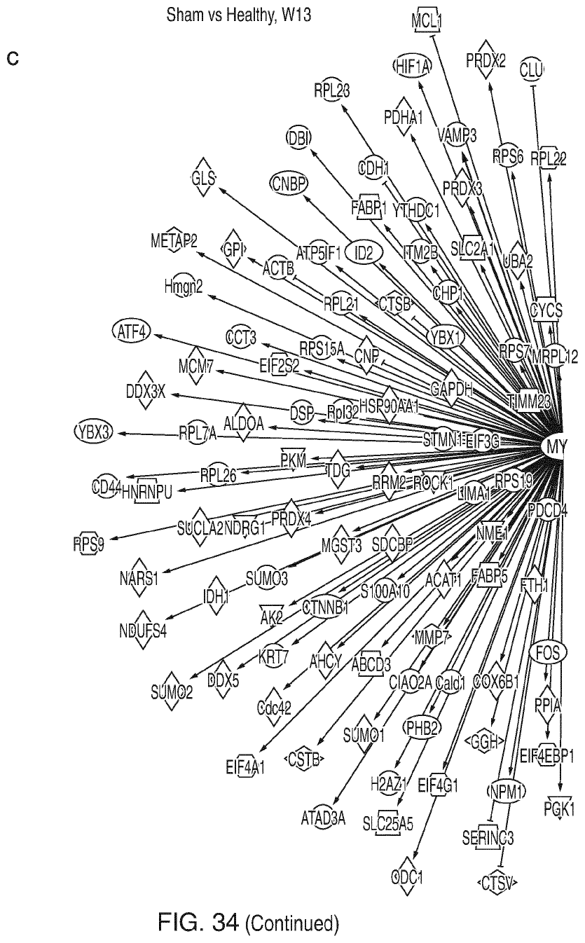

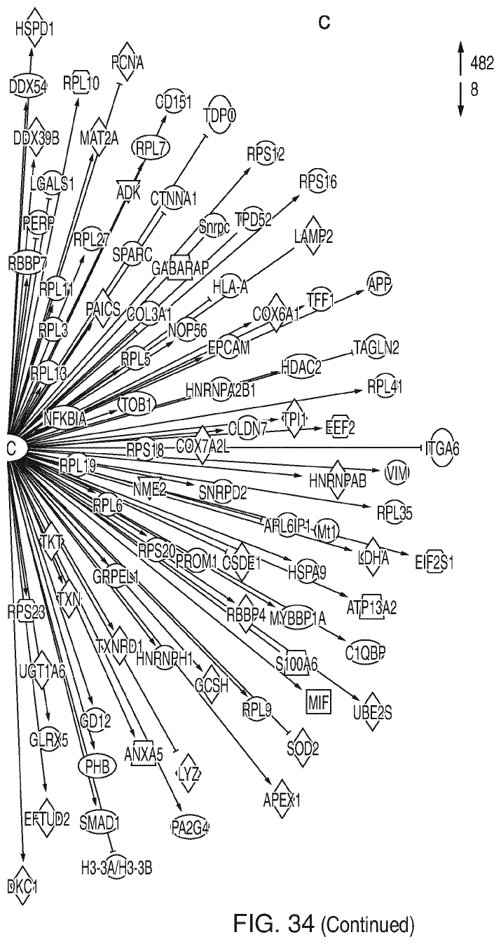

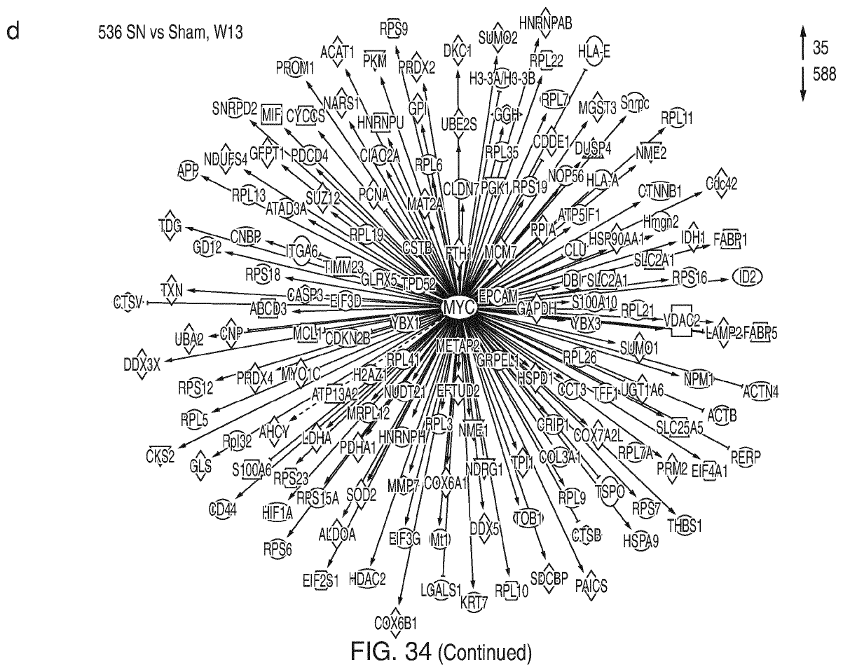

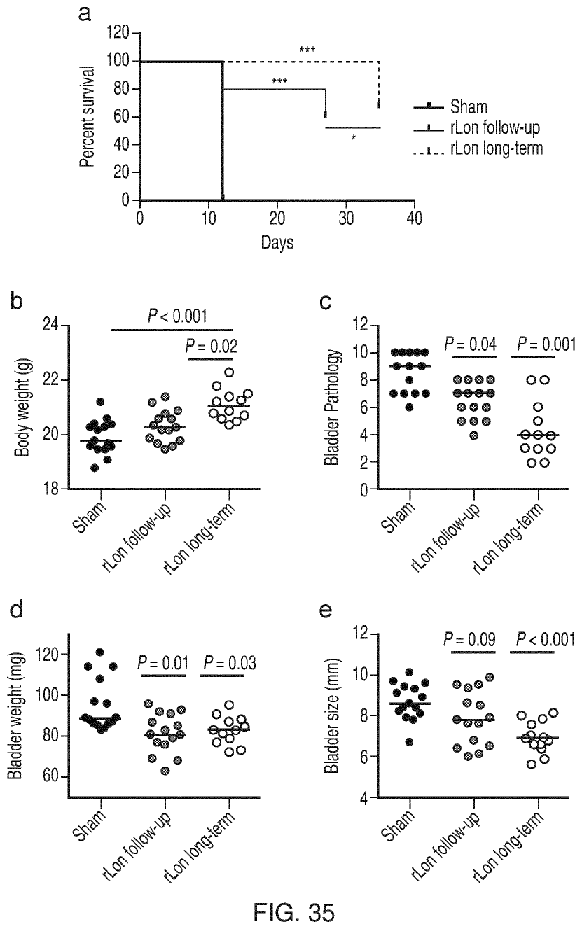

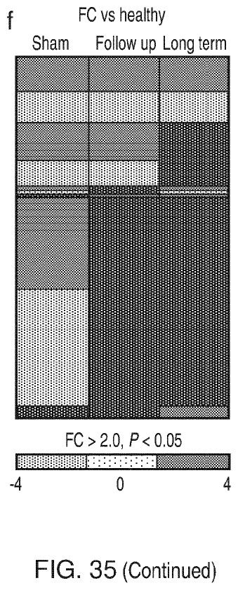

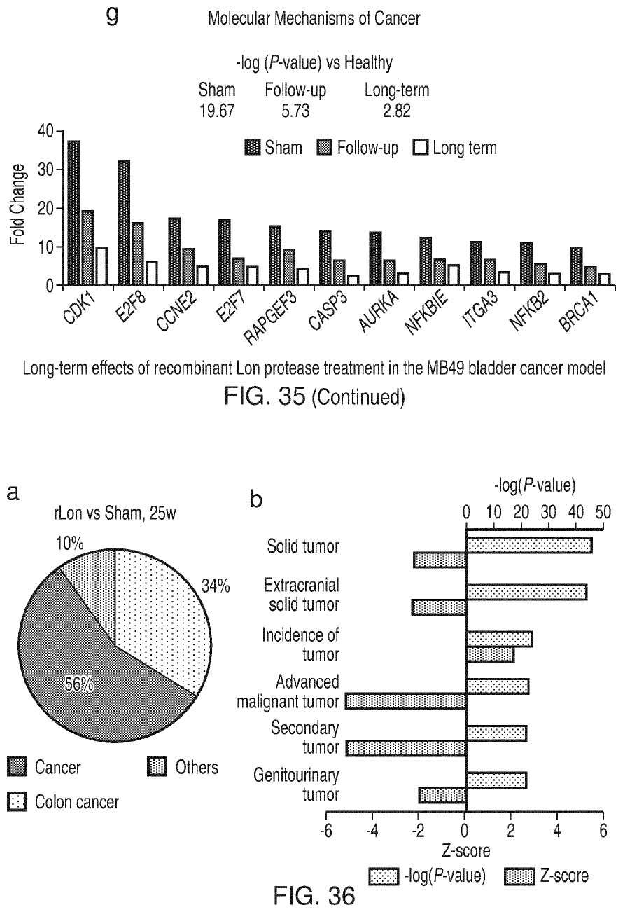

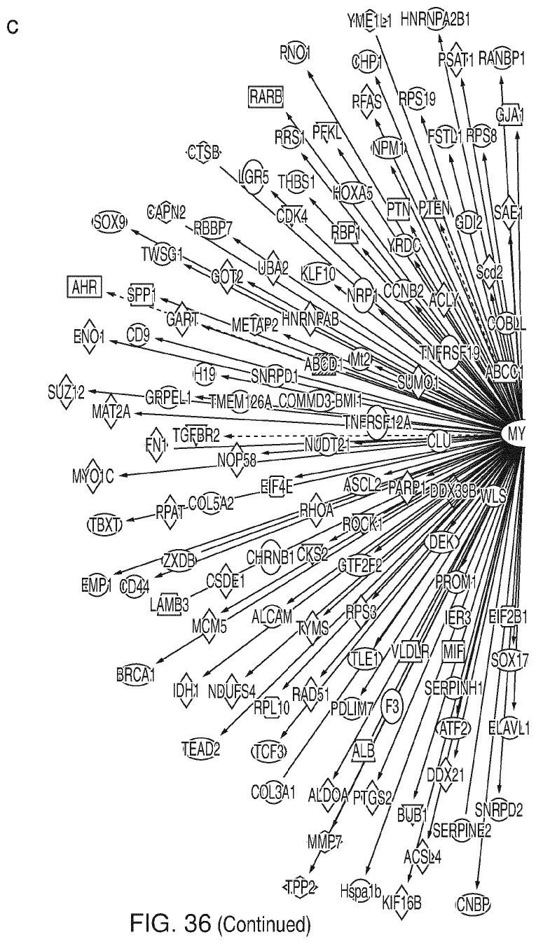

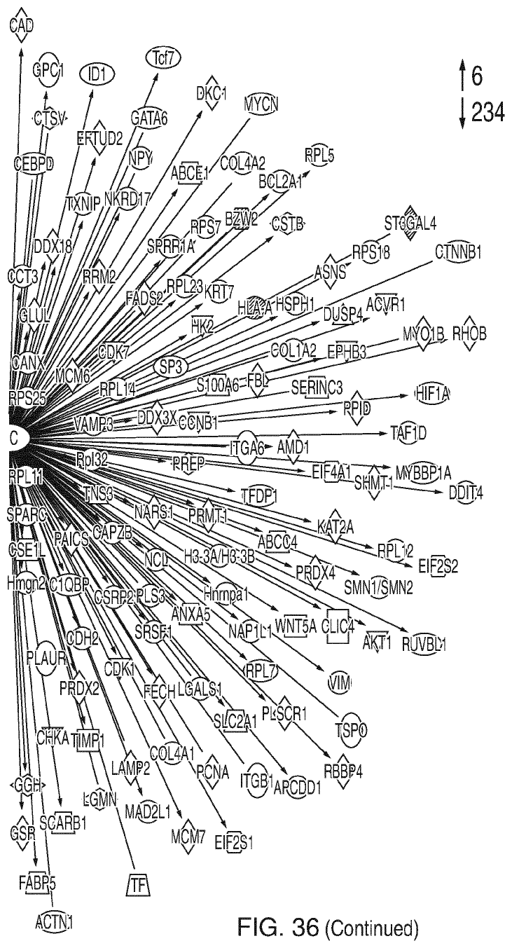

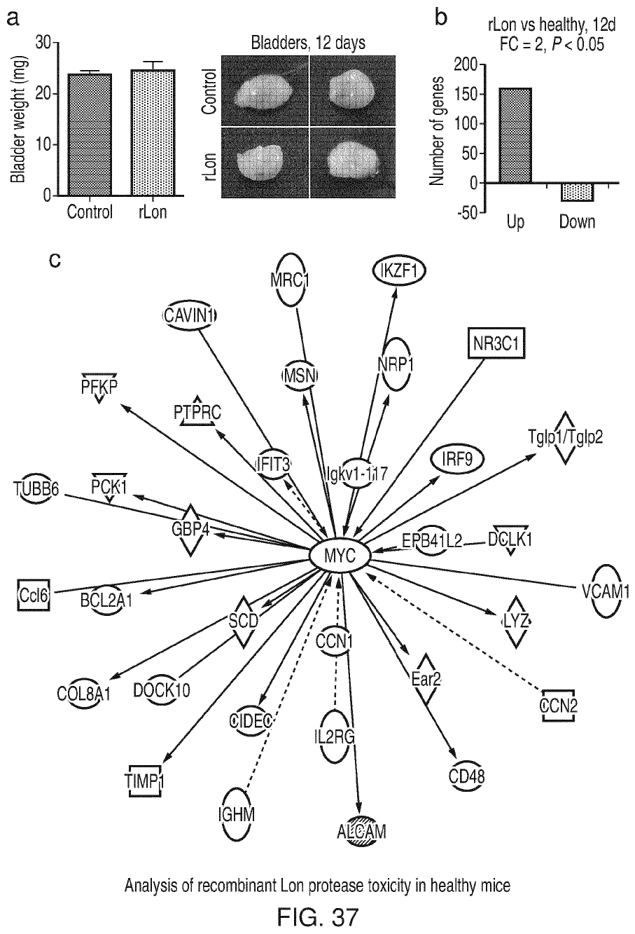

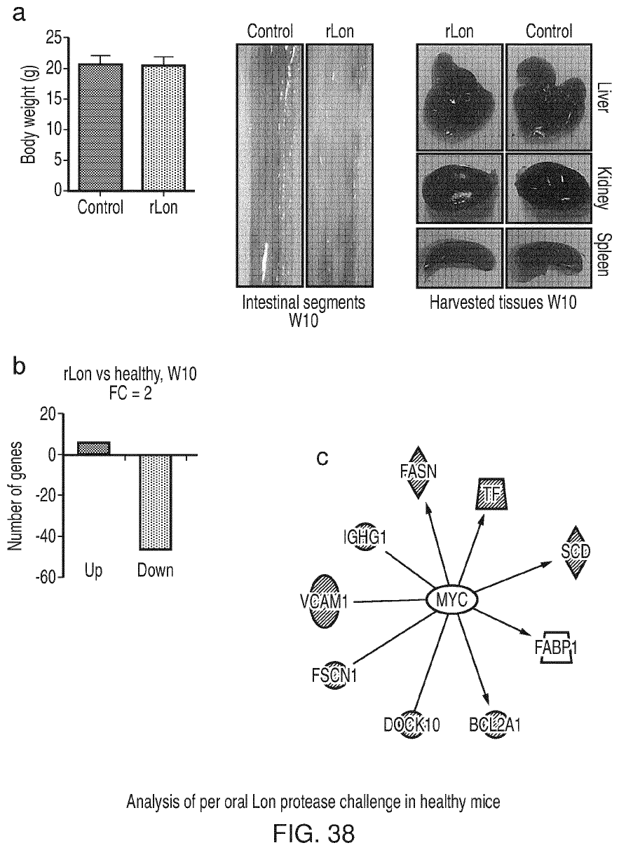

FIG. 21 shows effects of infection on cell viability. (Control experiment for FIG. 15a-c)

A498 cells were infected with E. coli 83972 (ABU), E. coli CFT073 (APN) or E. coli 536 (APN), (10⁵ CFU/ml). PBS served as a control. Cell viability was measured by (a) Lactate dehydrogenase (LDH) release and (b) Presto Blue levels.

FIG. 22 shows supplementary data for FIG. 16b.

A498 cells were infected with different E. coli 536 deletion mutants (PAI I₅₃₆ to PAI V₅₃₆ and PAI I-II₅₃₆) and screened for effects on cellular c-MYC levels (10⁵ CFU/ml, 4 hours). a, Low magnification confocal images. b, Quantification of fluorescent intensity from (a). Data is presented as mean±SEM from n=3 experiments and analyzed by paired t-test. **P<0.01, ***P<0.001.

FIG. 23 shows growth curve analysis of bacterial strains used in example 5.

A single colony from each E. coli strain or mutant was cultured in Luria Broth in a shaking incubator at 37° C. OD₆₀₀ was measured at 1, 2, 3, 4, 6 and 8h. All growth curves were replicates, two experiments per strain.

FIG. 24 shows bacterial proteins in supernatants of E. coli 536 and 536ΔPAI I.

a, Comparative Mass spec analysis of supernatants from E. coli 536 and PAI I₅₃₆. Five bands, abundant in E. coli 536 supernatant (SN) (bands 1-5, Coomassie staining), were excised, processed by in-gel digestion and analyzed by LC-MS/MS. b, The 10 most abundant proteins from each excised band are listed. c, A498 kidney epithelial cells were pre-treated with inhibitors of ATPase or GTPase driven endocytosis (Quercetin [25 μM] and Dynasore [30 μM] respectively) for 30 minutes prior to recombinant Lon protease (rLon) treatment (8 μg/ml, 4 hours). Confocal images show reduced Lon protease staining and uptake. d, Quantification of Lon protease staining in 50 cells (n=3 experiments). Data is presented as mean±SEM and analyzed by two-tailed t-test. *P<0.05. e, Protease digestion of E. coli 536 supernatant removed the MYC inhibitor, confirming that the soluble bacterial MYC inhibitor is a protein. Supernatants were pre-treated with increasing concentrations of trypsin (0-5 μg/ml, 30 minutes) before being added to A498 cells (4 hours, Western blot analysis). Trypsin was used as a negative control. f, Binding of recombinant Lon protease to recombinant c-MYC in a dose-dependent manner.

FIG. 25 shows peptide fragments from c-MYC protein by LC-MS/MS. (Supplementary data for FIG. 17c).

In the control sample 23 specific peptides corresponding to 51% of the sequence were detected. In rLon degraded c-MYC, 10 specific peptides corresponding to 23% of the sequence were found.

FIG. 26 shows CK1α1-dependent MYC degradation

a, Genes involved in proteasome degradation were upregulated in E. coli 536 infected A498 kidney epithelial cells, including CSNK1A1 and PLK3. b, CSNK1A1 expression was increased by E. coli 536 infection as well as E. coli 536 culture supernatants (SNs) (n=4 experiments). c, CK1α1 activation, defined by a redistribution from the nuclei to characteristic cytoplasmic puncta. Scale bar, 10 μm. d, Binding of CK1α1 to c-MYC shown by co-immunoprecipitation, using recombinant c-MYC and lysates from infected cells (n=3 experiments). e-f, Reversal of c-MYC inhibition by the CK1α1 inhibitor IC261 (30 minutes pre-treatment) by e, confocal imaging (Scale bars=10 μm) and quantification (black=control, red=E. coli 536) f, Western blot analysis (n=4 experiments). g, To inhibit Ca²⁺ fluxes, cells were pre-treated with EGTA (50 with μg/ml, 30 minutes) prior to stimulation with supernatants from 536 WT or 536Dhly^{1/2}. EGTA pre-treatment inhibited the activation of CK1α1. Data is represented as mean±SEM and analysed by paired t-tests. *P<0.05, **P<0.01, ***P<0.001.

FIG. 27 shows the role of hly I and hly II for MYC inhibition. Control experiments for FIG. 25.

a, Quantification of c-MYC levels in cells infected with E. coli 536 or E. coli 536Dhly^{1/2} (top panels) or exposure to bacterial supernatants (bottom panels). b, Quantification of c-MYC fluorescence in A498 kidney epithelial cells (n=2 experiments, compared to uninfected cells). Green=c-MYC; blue=nuclei. Scale bar, 10 μm. c, Western blot analysis of the corresponding cell extracts. d-f, Removal of molecules >100 kDa from E. coli 536 supernatant by molecular size filtration. The molecular size of the α-hemolysin (HlyA) protein is 109 kDa, predicting that HlyA should be excluded from the <100 kDa fraction, which still inhibited c-MYC in human cells, as shown by confocal imaging in (d-e) and Western blots in (f-g), Fractions <100 kDa did not affect cell viability quantified by ATP lite (N.S., compared to untreated cells, n=3 experiments). Data is presented as mean±SEM and analyzed by student's t-test. *P<0.05, **P<0.01, ***P<0.001.

FIG. 28 shows Hemolysin- and Lon protease release correlates with c-MYC degradation.

The clinical ABU and APN isolates from the collection shown in FIG. 15d was phenotypically characterized on blood agar and by western blot analysis. a, An association between Hly production and c-MYC degradation was detected. b, An association between Lon protease release and c-MYC-degradation was detected. c, Significant reduction in c-MYC levels in strains expressing both Lon and Hly in APN strains in FIG. 15d. d, No association between c-MYC levels in strains expressing both Lon and Hly in ABU strains in FIG. 15d. Data is presented as mean±SEM and analyzed by Mann-Whitney U-test. *P<0.05.

FIG. 29 shows the effects of hly^{1/2} and Ion deletions on transcriptional MYC regulators MAX, MYB and CEBPD. Control experiments for FIG. 18.

A498 kidney epithelial cells were infected with E. coli 536, 536 Dhly^{1/2} or 536 Dlon (10⁵ CFU/ml, 4 hours) a, d, g, MAX levels were not affected. b, e, h, c-MYB levels were reduced by E. coli 536, and Dlon but not affecting mRNA levels. c, f, i, CEBPD levels were only affected by E. coli 536. Confocal images (target proteins=green, nuclei=blue). d-f Quantification of images in (a-c). d, MAX, e, c-MYB and f, C/EBP-δ staining intensity compared to PBS. g-i, (g) MAX, (h) c-MYB and (i) CEBPD mRNA levels quantified by qRT-PCR, PBS served as control. Data is presented as mean±SEM and was analyzed by paired t-test (n=2-3 experiments). *P<0.05, **P<0.01, ***P<0.001.

FIG. 30 shows c-MYC inhibition in a variety of tumor cell lines. Control experiment FIG. 19.

a, A498 cells were infected with E. coli 536, PAI I₅₃₆, E. coli 536 Dhly^{1/2} or E. coli 536 Dlon (10⁵ CFU/ml, 4 hours), or treated with supernatants. WB of c-MYC levels. b, Cell viability as determined by LDH release after in vitro infection of A498 kidney epithelial cells. c, No change in viability in cells exposed to increasing concentrations of recombinant Lon protease (rLon) determined by LDH, ATP lite or Presto Blue.

FIG. 31 shows the lack of effect of E. coli PAI I₅₃₆ supernatants. Control experiment for FIG. 19.

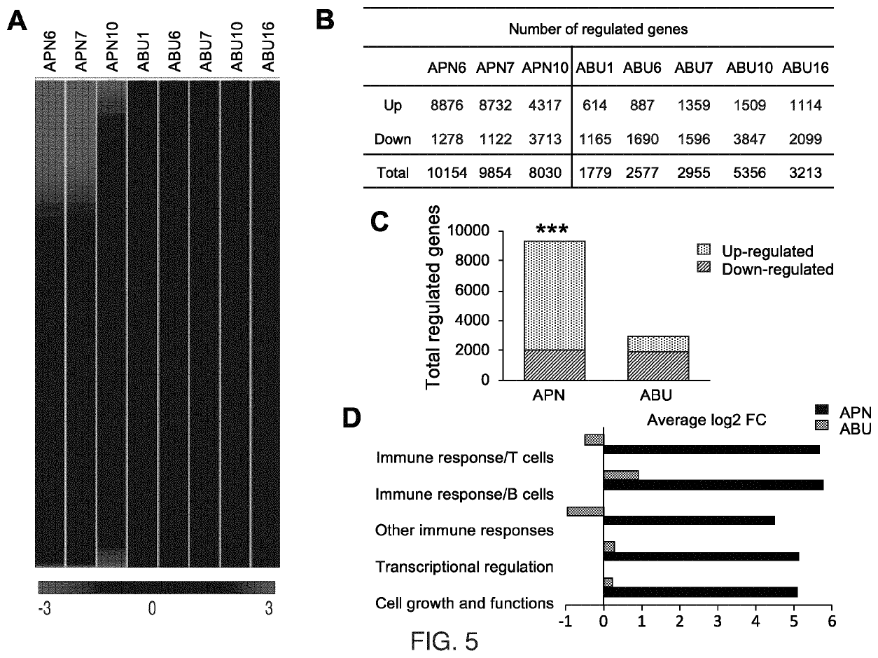

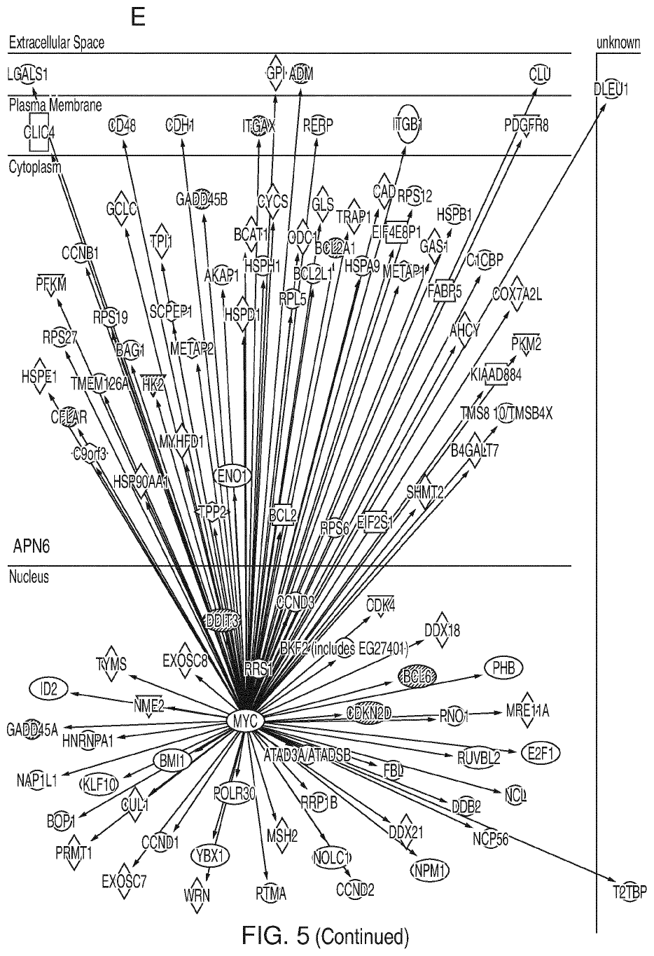

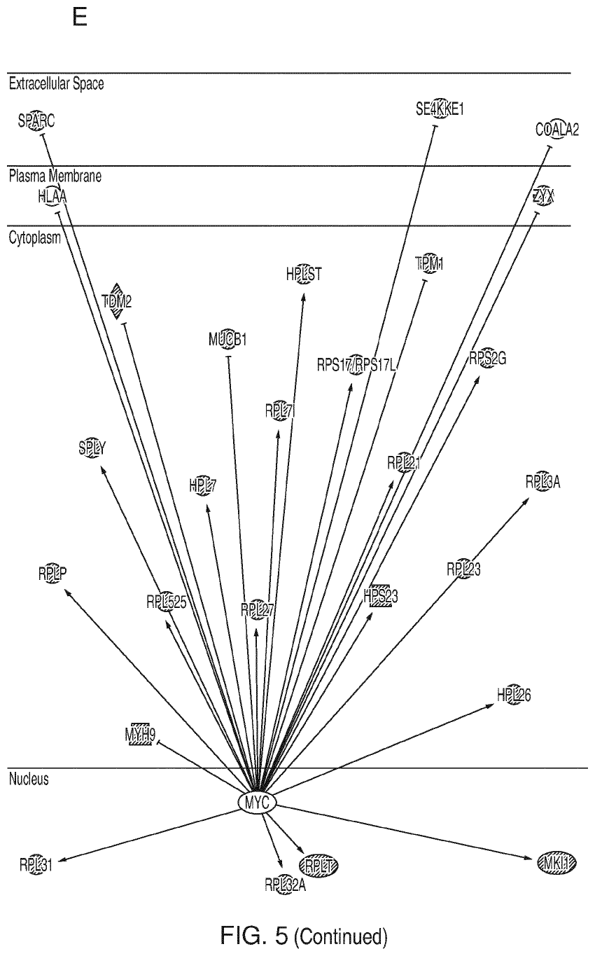

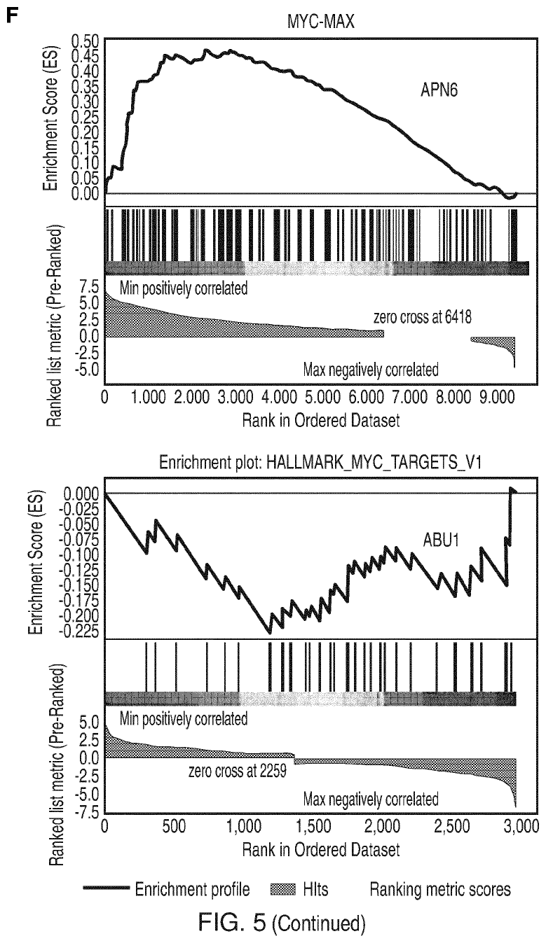

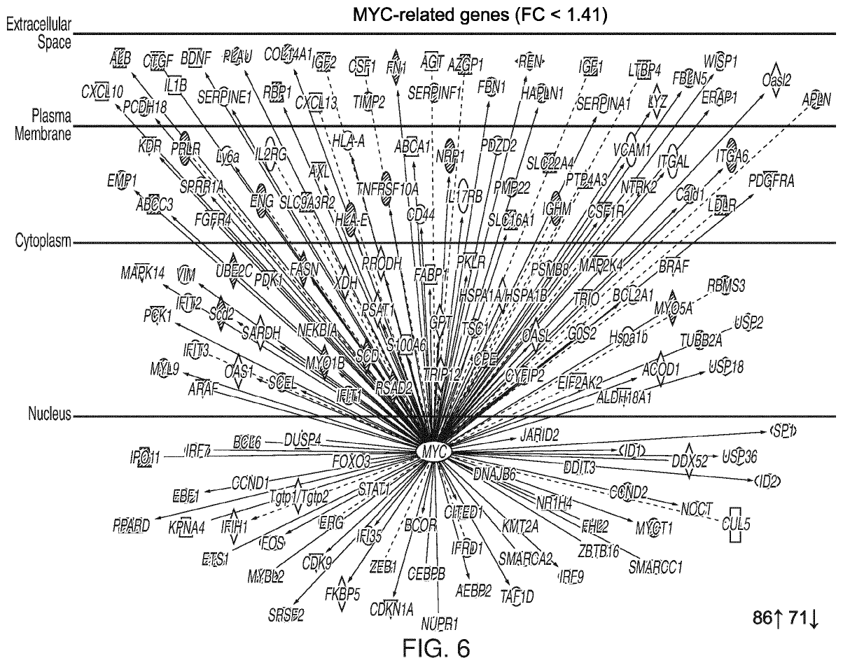

a, Tumor development was compared on day 12 between mice receiving E. coli 536 or PAI I₅₃₆ supernatants (SN). Bladder size and b, Tumor size, quantified from whole bladder tissue mounts, representative images from two mice. c, c-MYC expression was reduced in mice treated with E. coli 536 supernatant compared to mice receiving PBS or PAI I₅₃₆ supernatant. d, Western blot analysis of whole bladder tissue extracts in (c) (n=5 per group). e, Transcriptomic analysis of whole bladder RNA. The shift from a disease-specific towards a healthy tissue profile in E. coli 536 treated mice was not seen in the PAI I₅₃₆ group. f, Quantification of bladder weight, bladder size, tumor size and MYC staining. Each dot indicates a separate mouse and the lines represent medians. Data from sham and E. coli 536 supernatant treated mice is also included in main FIG. 5. The data was analysed by Mann-Whitney U-test.

FIG. 32 shows gene expression analysis of tumor-bearing mice treated with E. coli 536 or E. coli PAI I₅₃₆ supernatant

a, Heat map of regulated genes in tumor bearing mice on day 12 treated with E. coli 536 supernatant (SN)-, E. coli PAI I₅₃₆ supernatant- or sham treated controls, compared to healthy bladders. A marked reduction in the number of regulated genes was observed in the E. coli 536 supernatant group (Heat map, FC >2.0, n=1 mouse per group). b, Activation of MYC-dependent network by E. coli PAI I₅₃₆ supernatant treated and Sham mice. c, Further analyses showed strong inhibition of cancer pathways in the E. coli 536 supernatant treated mice, compared to the sham or E. coli PAI I₅₃₆ supernatant groups.

FIG. 33. Gene expression analysis of tumor-bearing mice treated with E. coli 536 supernatant for 12 days.

a, Significantly regulated gene categories, showing strong regulation of Cancer related categories. b, Heat map of regulated genes in tumor bearing mice treated with E. coli 536 supernatant (SN)- or recombinant Lon protease (rLon)-treated mice and the sham group, compared to healthy bladders. c, Principle Component Analysis (PCA) plot showing a shift from disease to a healthier phenotype in rLon and E. coli 536 supernatant-treated mice. d, Cancer functions regulated in the E. coli 536 supernatant treated mice, compared to the sham group. e, MYC-related network in E. coli 536 supernatant treated mice compared to the sham group. f, Biofunctions regulated by E. coli 536 supernatant or rLon compared to healthy bladders. g, Corresponding Z-scores for significantly regulated functions. Data from rLon compared to Sham is also included in FIG. 19.

FIG. 34. Control experiment for FIG. 20.

Therapeutic effect of E. coli 536 supernatant (SN) treatment in APC^{Min/+} mice compared to the sham group, showing a reduction in intestinal polyps in a, 13 weeks old mice, b, 22 weeks old mice. Each dot represents individual mice and the line show the median and was analyzed by Mann-Whitney U-test. c, MYC-dependent gene network analysis in 13 weeks old APC^{Min/+} mice compared to healthy C57BL/6 WT mice, confirming the c-MYC dependence of intestinal tumorigenesis in APC^{Min/+} mice. d, Inhibition of MYC-dependent gene networks in mice treated with E. coli 536 supernatant, compared to sham treated mice. Data from rLon compared to Sham is also included in FIG. 20.

FIG. 35 shows long-term effects of recombinant Lon protease treatment in the MB49 bladder cancer model

a, Kaplan Meyer plot showing increased survival in recombinant Lon protease (rLon) treated mice. b, Body weight in sham mice (12 days), rLon treated mice without further intervention (27 days, follow-up) and rLon treated mice with twice weekly treatment until sacrifice (35 days, long-term). c-e, Changes in bladder pathology, bladder weight and bladder size in rLon treated mice. f, Heatmap of regulated genes in the sham group, rLon treated mice follow-up and long-term groups compared to healthy mice (n=2). g, Genes associated with the Molecular Mechanisms of Cancer pathways were strongly inhibited by rLon treatment. The activation was increasingly lower in follow-up and long-term treated mice, respectively.

FIG. 36 shows gene expression analysis. Effects of recombinant Lon protease treatment in APC^{+/min} mice after long-term follow-up

a, Pie chart of significantly regulated gene categories by recombinant Lon protease (rLon) treatment. b, Details of cancer-related functions regulated in rLon treated mice compared to the sham group (W25). c, Network showing inhibition of MYC-related genes. The MYC inhibition was maintained in the long-term follow-up group.

FIG. 37 shows analysis of recombinant Lon protease toxicity in healthy mice.

a, C57BL/6J WT mice were exposed to recombinant Lon protease (rLon) intravesically for 12 days. No change in bladder weight or macroscopic appearance of the bladder was detected. b, Few genes were regulated in bladders from healthy rLon treated mice (n=2 rLon, 1 healthy). c, MYC expression was not regulated. A small number of MYC-dependent genes was regulated in bladders from healthy rLon treated mice (33 activated, 1 inhibited).

FIG. 38 shows analysis of per oral Lon pretease challenge in healthy mice.

a, C57BL/6J WT mice were exposed to recombinant Lon protease (rLon) per-orally twice daily for 14 days. No change in body weight, or macroscopic appearance of the intestine, liver, kidney or spleen was detected. b, Gene expression analysis detected a lack of transcriptional activation in healthy rLon treated mice (n=3 rLon, 1 healthy). c, MYC expression was not regulated and few MYC-related genes were affected in healthy intestines after rLon challenge (1 activated and 8 inhibited genes).

### DETAILED DESCRIPTION

In experimental cancer models, c-Myc inhibition has already been shown to arrest cancer progression and even restore tissue integrity. Here, we identify a new molecular strategy for MYC inhibition, developed by pathogenic E. coli strains. Based on a combination of accelerated degradation and inhibition of gene expression, the bacteria effectively reduce MYC levels in infected cells and tissues.

### Example 1—Rapid Depletion of Renal c-MYC Levels Following Infection

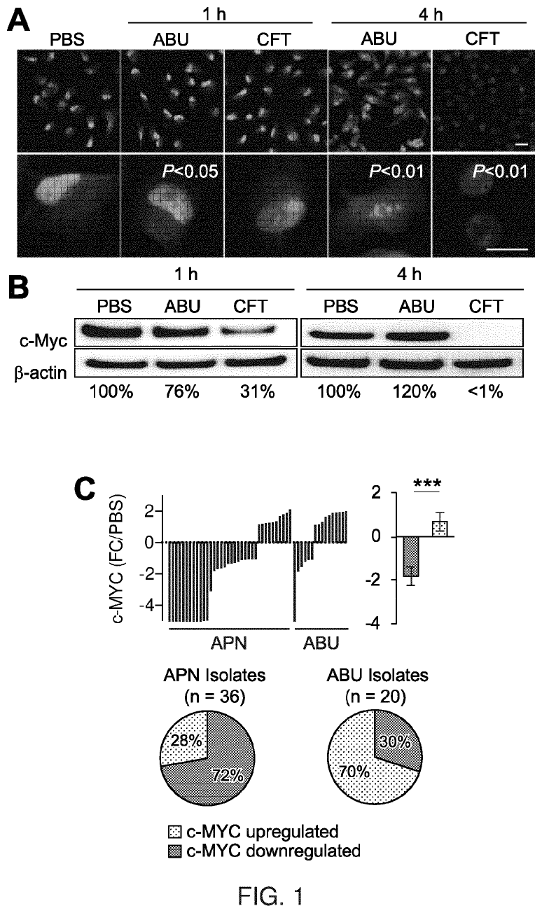

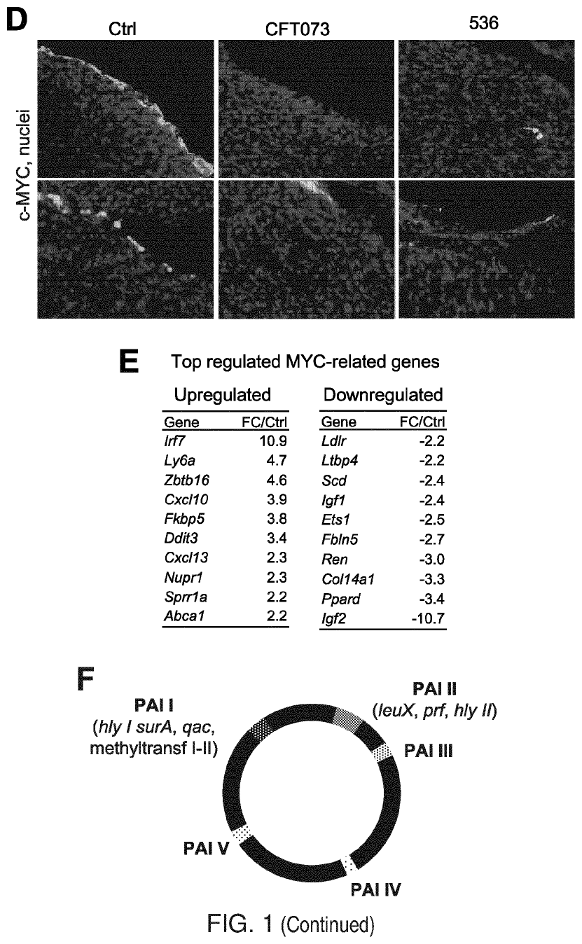

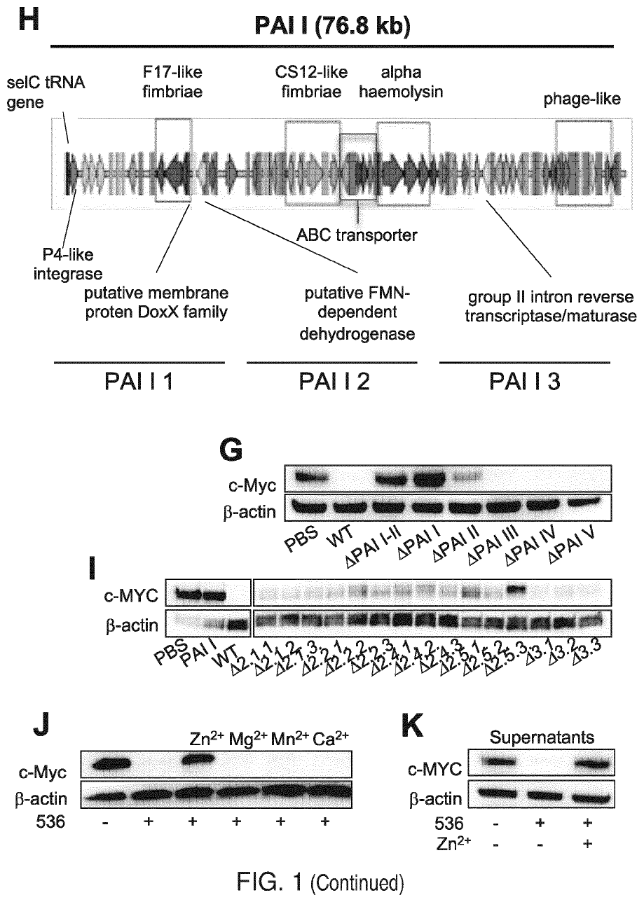

To investigate if renal c-MYC expression is modified by infection, we selected human kidney epithelial cells, which are the first to be targeted when bacteria ascend into the renal pelvis. A rapid, time-dependent reduction in c-MYC protein levels was detected after infection with E. coli CFT073; a uropathogenic strain (FIGS. 1A-B). The ABU strain E. coli 83972 was inactive, suggesting that MYC inhibition might be virulence-related. Cellular c-MYC levels were indeed reduced by 72% of well-characterized uropathogenic isolates (n=36) compared to 30% of ABU strains (n=20, P<0.001) (FIG. 1C), confirming this hypothesis.

c-MYC staining was also reduced in vivo, after intra-vesical infection of mice with CFT073 (P<0.05, FIG. 1D). Myc-related genes were suppressed (n=157), including regulators of gene expression, cancer pathways, renal cell proliferation and tissue repair (FIG. 1E, FIG. 52). Igf2, which is essential for renal growth and development, was strongly inhibited (fold change −log¹⁰³⁶). Irf7 was the top up-regulated gene (fold change log^{10.9}) consistent with its key role in infection-induced kidney pathology (FIG. 1D), suggesting a mechanism of renal growth retardation and tissue damage in children with acute pyelonephritis (APN). A second uropathogenic strain, E. coli 536, reproduced these in vivo effects.

### Example 2—Gene Deletion Strategy to Identify Bacterial c-MYC Regulators/Inhibitors

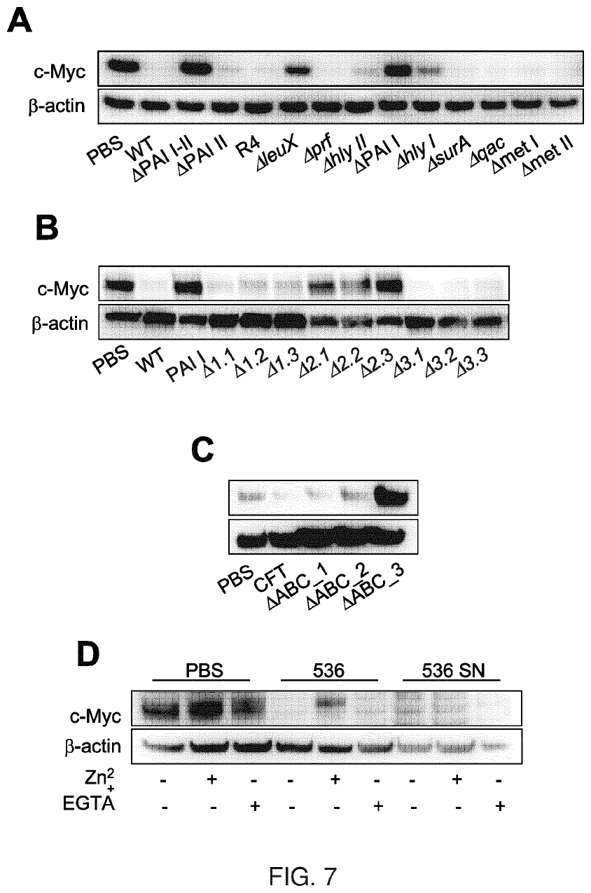

To address the mechanism of bacterial c-MYC inhibition, we selected E. coli 536, in which virulence genes and chromosomal pathogenicity islands (PAIs) have been extensively characterized (FIG. 1F). A screen of specific chromosomal pathogenicity island deletion mutants identified E. coli 536 ΔPAI I (536-114) as a “loss of function” mutant, suggesting that regulators of c-MYC are encoded on this island (FIG. 1G). Specific deletions of known virulence genes encoded on PAI I did not affect MYC inhibition, except for Δhly1 and ΔPAI II+ΔleuX, which showed intermediate phenotypes (FIG. 7).

By sequential deletions from the 5′ end of PAI I using lambda red homologous recombination, we identified a bacterial ABC metal ion transporter encoded on the PAI I 2.5.3 sub-fragment (ORF42-ORF46). The ABC transporter controlled MYC inhibition in E. coli 536 and by deletion of the corresponding ABC transporter in CFT073, which in this strain is located outside of PAI I, we confirmed its importance for bacterial MYC inhibition (FIGS. 1H-I). In a metal ion screen, ZnCl₂ was shown to abolish c-MYC inhibition, suggesting that the bacterial ABC transporter regulates the bacterial c-MYC inhibitor(s) by controlling Zn²⁺ fluxes (FIGS. 1J-K).

The inhibitory effects of E. coli 536 and CFT073 were reproduced, using bacterial culture supernatants (RPMI, 4 hours), suggesting that bacteria release molecules to achieve this effect. ZnCl₂ abolished this effect indicating that Zn²⁺ regulates the production of the bacterial MYC inhibitors. In contrast, ZnCl₂ did not affect the execution of MYC suppression in host cells (FIG. 53).

### Example 3—Mechanism of MYC Degradation in Host Cells

Transcription factors are rapidly degraded and c-MYC has an estimated half-life of around 30 minutes. We have obtained convincing evidence that bacteria accelerate MYC protein degradation and inhibit MYC expression.

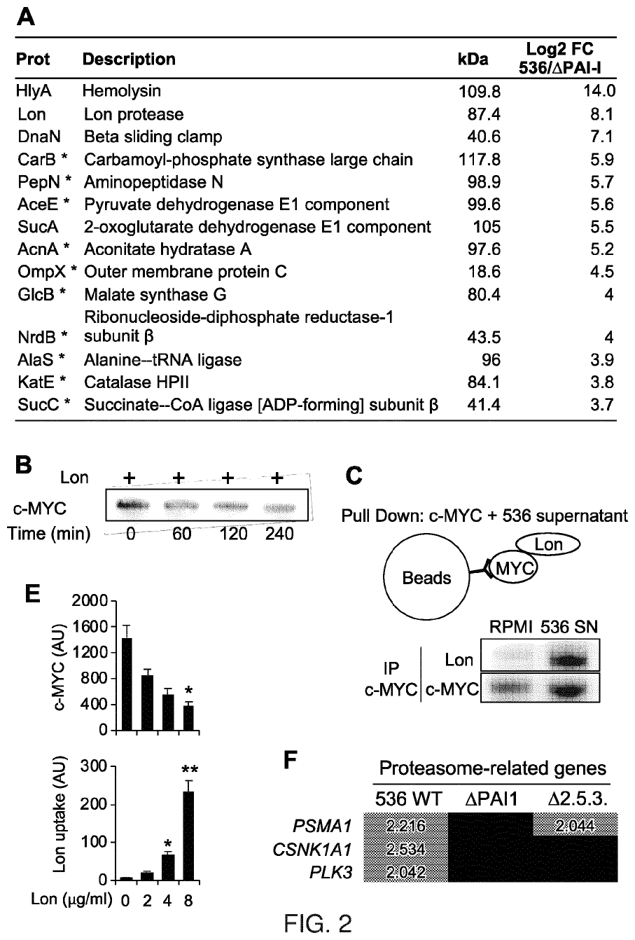

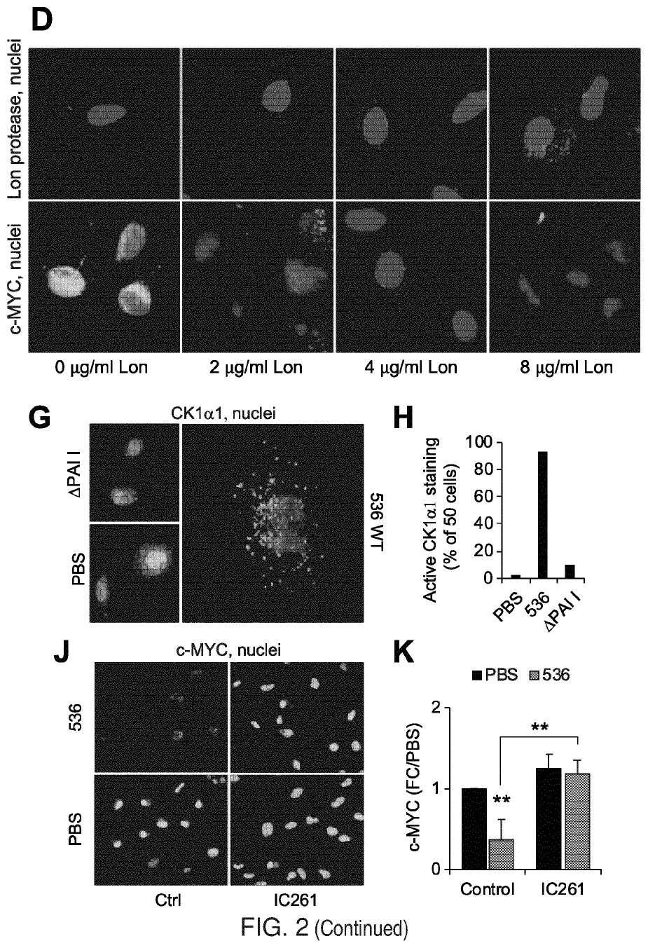

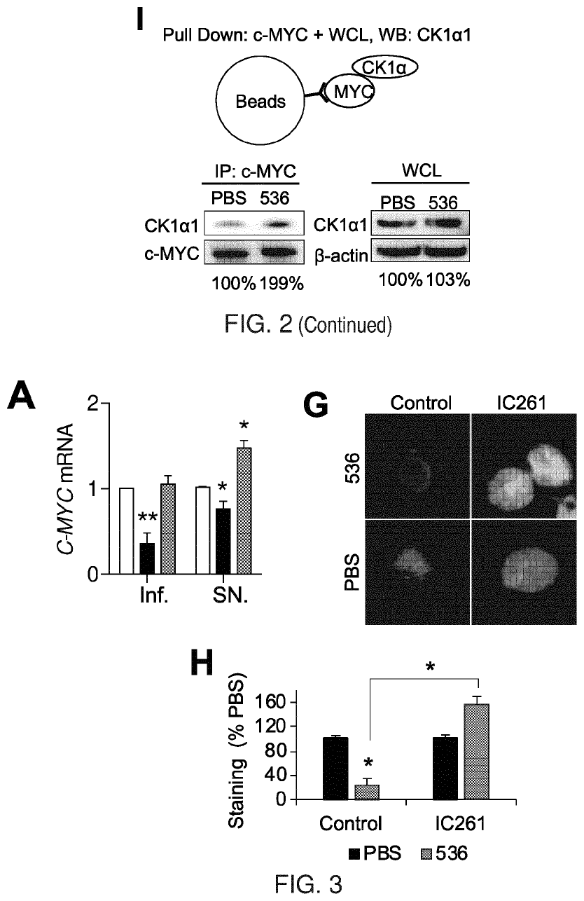

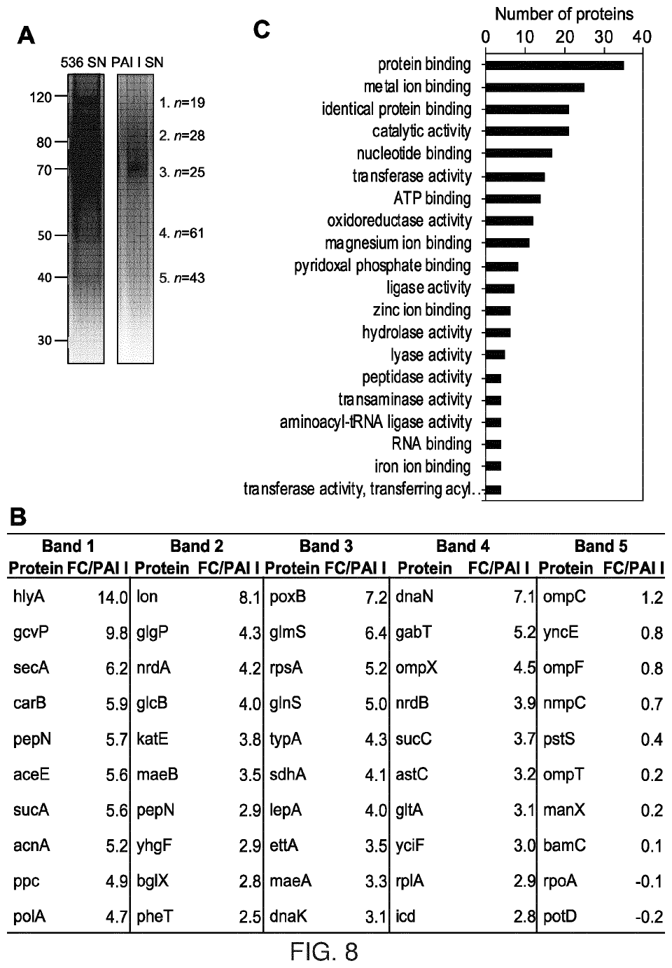

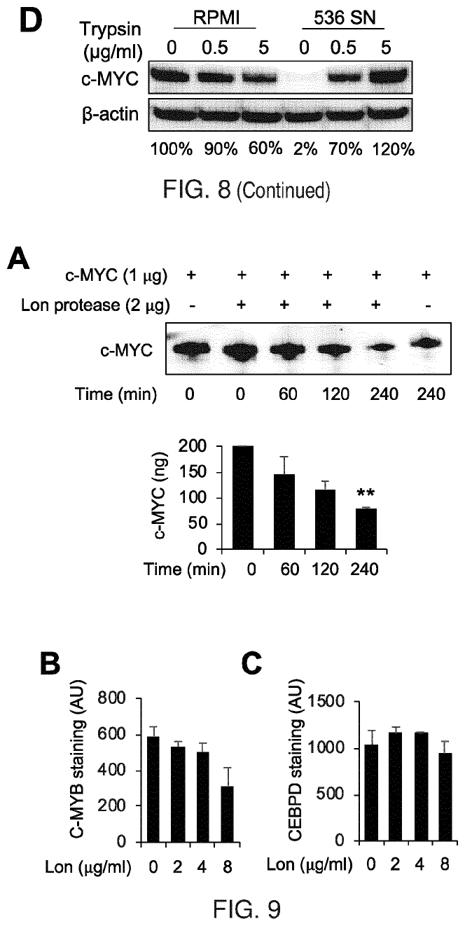

Two separate mechanisms of MYC degradation were defined. By comparative mass spectrometry of supernatants from E. coli 536 and ΔPAI I loss of function mutant, we identified the Lon serine protease as an abundant constituent of E. coli 536 supernatant (FIG. 2A, FIG. 8). Recombinant bacterial Lon protease degraded c-MYC in vitro, in a time-dependent manner (FIG. 2B, FIG. 9). Furthermore, cells exposed to recombinant Lon protease showed a dose dependent increase in Lon staining, suggesting invasion of Lon into the cytoplasm of host cells and some nuclear translocation. In parallel, a dose-dependent decrease in MYC staining was observed, suggesting a direct effect on cellular MYC levels. This was confirmed by co-immunoprecipitation of Lon from total cell extracts, using MYC-coated beads (FIGS. 2C-E).

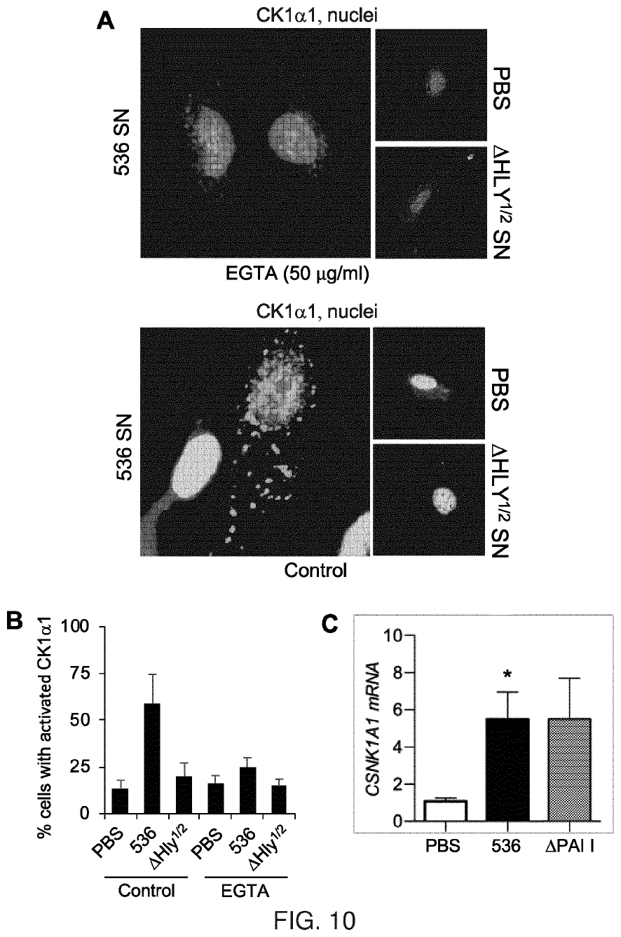

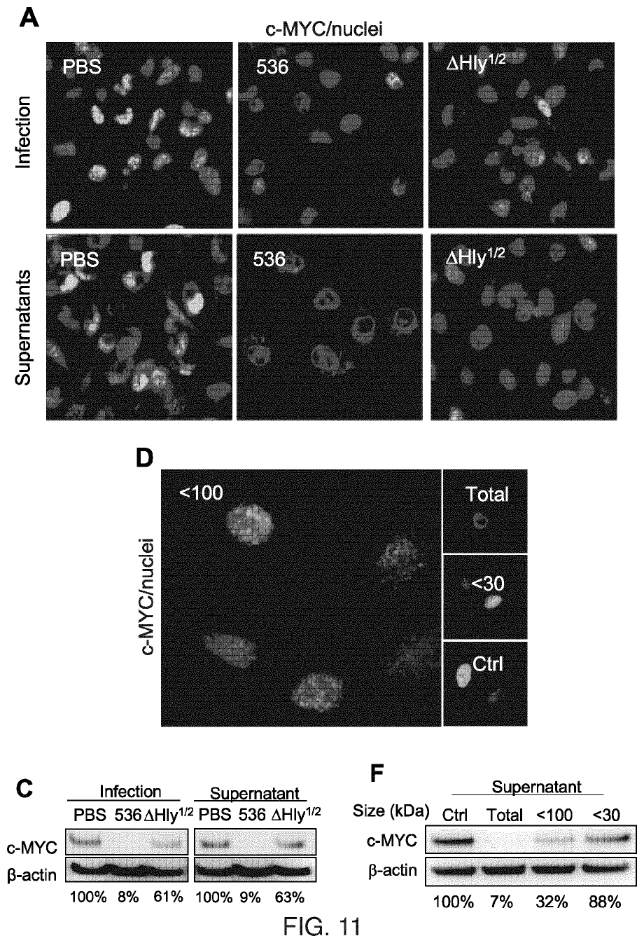

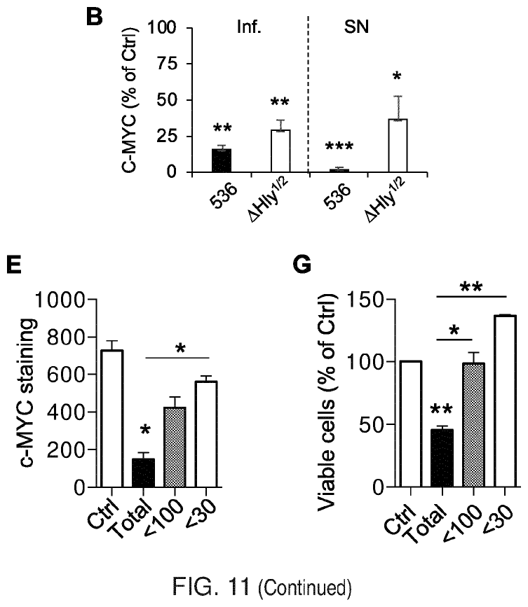

By gene expression analysis of infected cells, we also observed a set of proteasome related genes, which were inversely regulated, by E. coli 536 or ΔPAI I (FIG. 2F). CSNK1A1 expression was specifically upregulated in E. coli 536 infected cells. Casein kinase 1 alpha 1 (CK1α1), encoded by CSNK1A1, phosphorylates c-MYC at Serine²⁵² thereby marking the protein for proteasomal degradation (17). E. coli 536 infected cells showed a vesicular cytoplasmic staining pattern characteristic of CK1α1 activation (17) (FIGS. 2G-H). By co-immunoprecipitation, recombinant c-MYC was shown to bind CK1α1 in whole cell lysates (FIG. 2I) and c-MYC degradation was prevented by pharmacologic inhibition of CK1α1 (IC261) (18), linking CK1α1 activation to accelerated MYC degradation (FIGS. 2J-K). CK1α1 was activated by Ca²⁺ fluxes, induced by the bacterial alpha-hemolysin, known to be regulated by Zn²⁺ (FIGS. 10-11).

The results suggest that E. coli 536 accelerates c-MYC degradation by at least two distinct mechanisms, defined by uptake of the bacterial Lon protease and activation of the human CK1α1 kinase.

### Example 4—Bacteria Inhibit c-MYC Expression

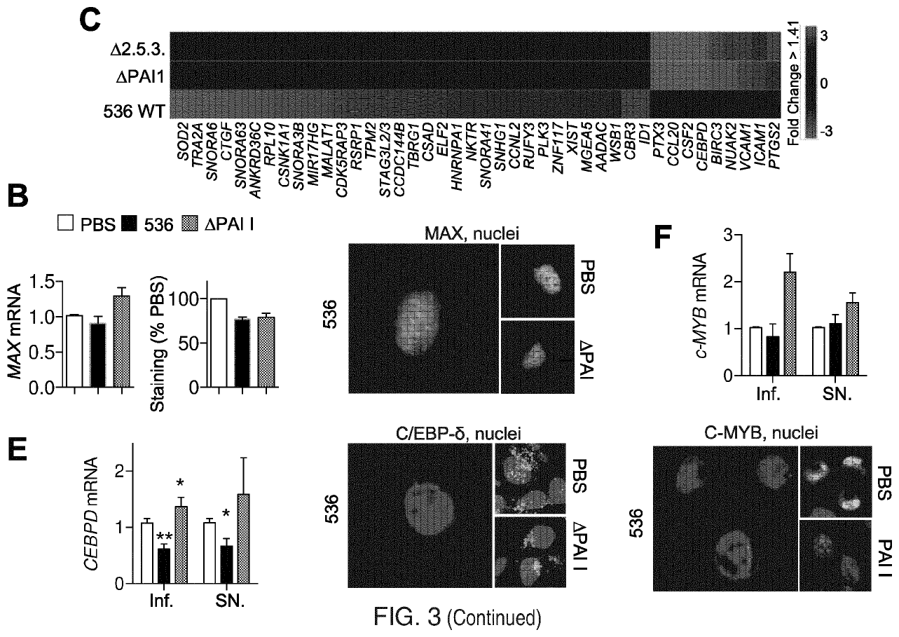

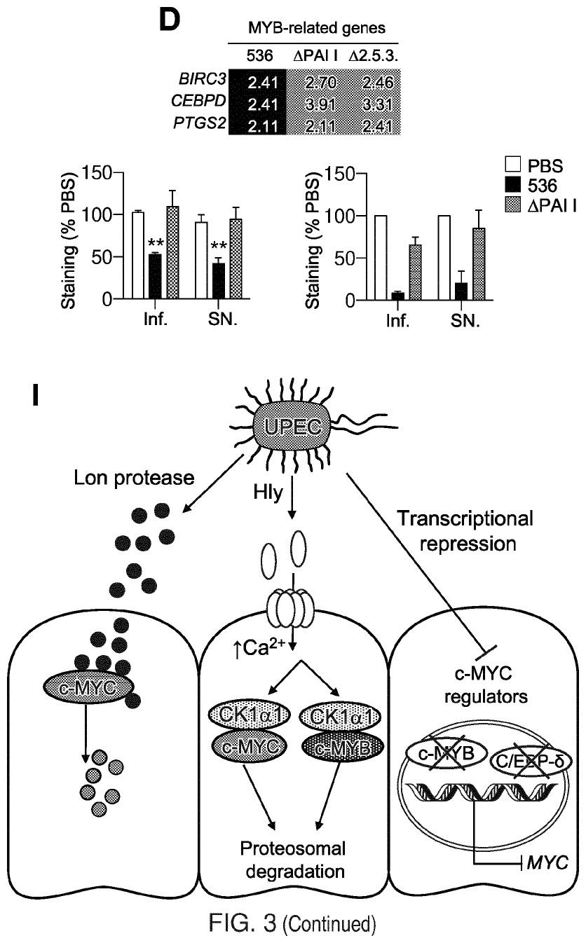

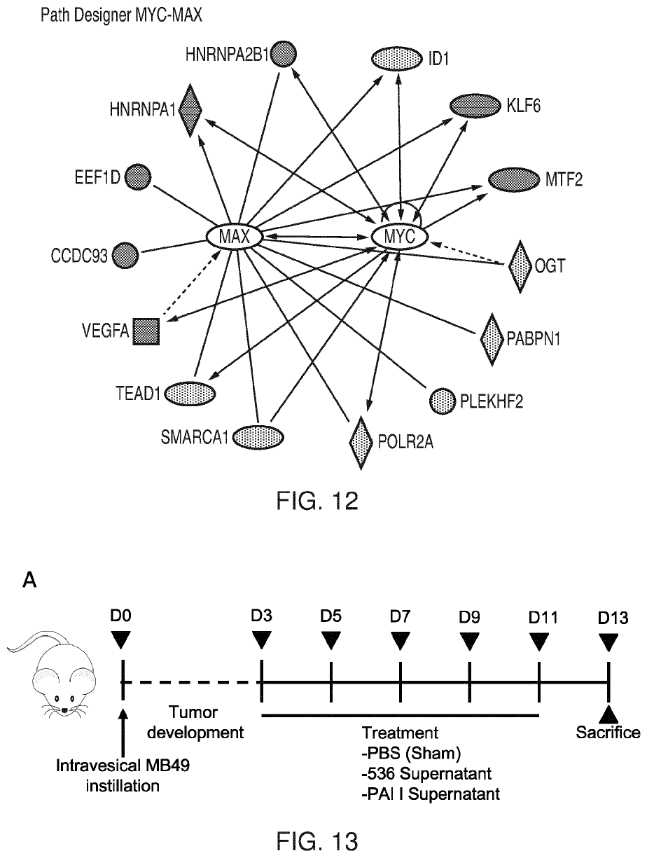

MYC expression was reduced in infected human kidney cells, suggesting that the bacteria also affect c-MYC expression and the supernatants reproduced these effects (4 hours, FIG. 3A). MYC works in concert with a large number of regulators, most prominently MYC associated factor X (MAX), forming the MYC-MAX complex, which activates transcription from a large number of promoters. In this study, MAX was not regulated at the transcriptomic level and there was no significant change in MAX staining in infected cells, suggesting that the bacteria mainly target MYC, possibly to limit range of biological effects (FIG. 3B, FIG. 12). It is interesting to speculate that MAX might have evolved, in part, to override bacterial MYC inhibition at sites of infection.

Transcriptomic analysis defined a network of MYB (myeloblastosis)-related genes, which was inhibited in E. coli 536 infected cells (FIGS. 3C-D). CEBPD (CCAAT enhancer binding protein delta) was transcriptionally repressed and CEBP-δ protein levels were reduced (4 hours, FIG. 3E). Cellular MYB protein levels were markedly attenuated, in a CK1α1 dependent manner (FIGS. 3F-H). ΔPAI I bacteria or supernatant did not inhibit CEBPD expression or affect MYB protein levels.

Biological Relevance

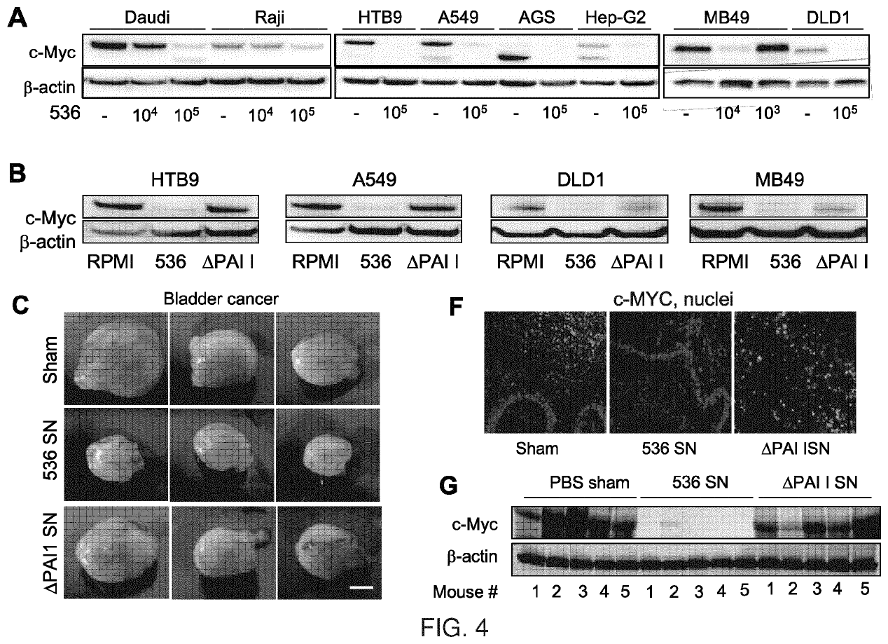

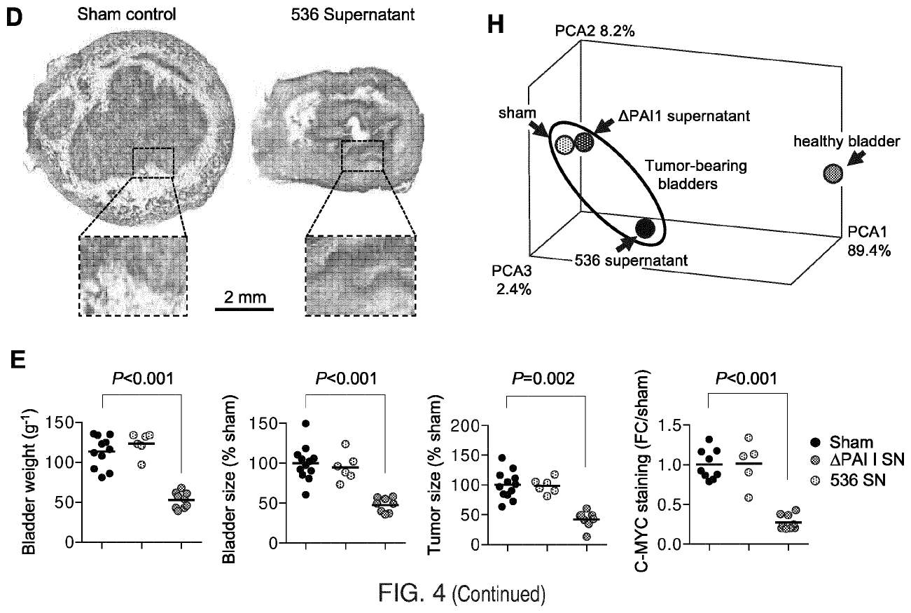

The MYC family of oncogenes is subject to “a bewildering assortment of genetic rearrangements” associated with many different tumors cell types. We therefore investigated, if the bacterial MYC inhibition mechanism is relevant also for cancer cells of other origins. E. coli 536 infection reduced cellular c-MYC levels in Burkitt lymphoma cells, where c-MYC was first discovered, as well as gastric (AGS) and hepatic (HEP-G2) carcinoma cells (FIG. 4A). Supernatants showed a similar effect on human bladder (HTB9), small airway (A549) and colon cancer cells (DLD1) as well as murine bladder carcinoma cells (MB49). The ΔPAI I mutant served as a negative control (FIG. 4B).

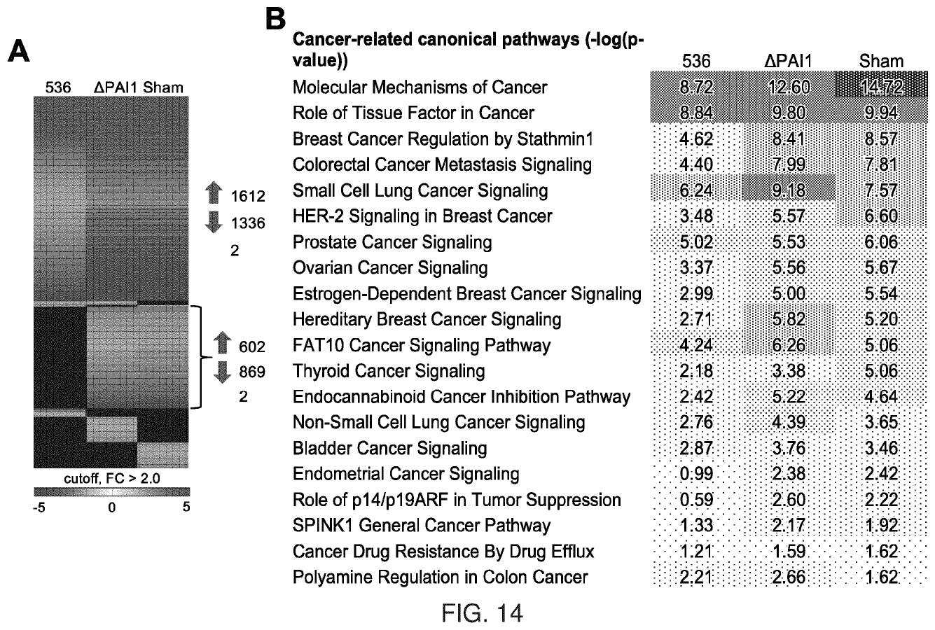

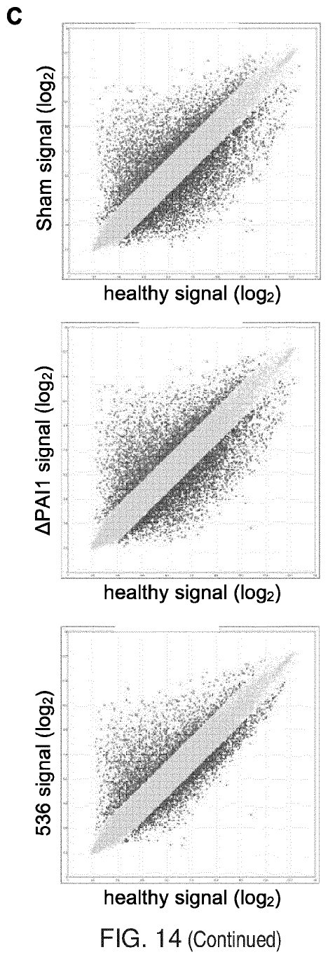

Therapeutic effects of the bacterial supernatant were demonstrated in the murine MB49 bladder cancer model. Bladder cancer was established in C57BL/6 mice, by instillation of MB49 cells and the treatment group received five intra-vesical instillations of E. coli 536 supernatant, ΔPAI I supernatant or PBS (sham) (FIG. 13). Sham-treated mice developed palpable tumors that altered the macroscopic appearance of the bladders as the tumor mass gradually filled the bladder lumen (FIGS. 4C-D). The E. coli 536 supernatant reduced tumor size, bladder weight and bladder size compared to sham-treated mice (FIG. 4E). In addition, c-MYC levels were reduced in tumor tissue, as shown by immunohistochemistry and western blots of whole bladder extracts (FIGS. 4F-G). By principal component analysis of genes expressed in bladder tissues, we detected a significant shift in the treatment group (E. coli 536 supernatant) from a disease-specific pattern (sham) towards a normal gene expression profile (healthy bladder), characterized by a marked reduction in cancer-related gene networks (FIG. 4H, FIG. 14). The ΔPAI I supernatant lacked efficacy in this model.

Materials and Methods for Examples 1 to 4

This study addressed the mechanism of c-MYC control by uropathogenic E. coli. The study was initiated when a transcriptomic screen of patients prone to acute pyelonephritis (APN) revealed elevated expression of MYC and MYC-related genes compared to patients with asymptomatic bacteriuria (ABU). As RNA was collected during an infection free interval, these differences were not likely to reflect a direct response to infection but a difference in baseline MYC activity. To understand the role of c-MYC during acute pyelonephritis, we therefore quantified c-MYC levels in in infected human kidney epithelial cells in vitro, and in kidneys from infected C57BL/6 WT mice. Both experiments showed a marked reduction in epithelial MYC levels, suggesting that the bacteria activate the cellular MYC degradation machinery and possibly suppress MYC expression. Screening of clinical isolated showed that MYC suppression was characteristic mainly of uropathogenic E. coli and not of the more commensal like ABU strains.

To identify bacterial factors responsible for c-MYC regulation, we used a systematic gene-deletion strategy, successively removing pathogenicity island genes from the E. coli 536 chromosome. MYC inhibition was regulated by genes on Pathogenicity island I and a Zn²⁺ -dependent ABC transporter was identified as a key regulator of the bacterial MYC-inhibitor, with partial effects of hly1. A list of potential bacterial MYC inhibitors was identified in bacterial supernatants by LC-MS/MS. The bacterial Lon protease was internalized by host cells and reduced cellular MYC levels in a dose dependent manner, possibly as a result of direct binding and proteolytic degradation of MYC. was detected in vitro one of the potential MYC-inhibitors, and by addition of recombinant Lon protease to cells, c-MYC levels were reduced, and Lon protease was showed to effectively degrade c-MYC in vitro.

Relevance to different forms of cancer was supported by a reduction in cellular MYC levels after infection or challenge with bacterial supernatants. Finally, therapeutic efficacy was documented in a murine bladder cancer model, treating tumor-bearing mice with bacterial supernatants before evaluating the treatment effects on the tumors by pathological assessment and genome-wide transcriptomic analysis.

Bacterial Strains

APN (n=36) isolates and ABU (n=20) isolates were isolated during a prospective study of childhood UTI in Gothenburg, Sweden. The prototype strains E. coli CFT073 (O6:K2:H1) (28), E. coli 536 (O6:K15:H31) and E. coli 83972 (OR:K5:H-) (31) were used as control strains. Bacteria was grown on trypsin soy agar (TSA) plates at 37° C. for 16 hours and was harvested in phosphate buffered saline (PBS, pH 7.2) and appropriately diluted for infection.

Sub-Cloning of E. coli 536 ΔPAI I

The deletion mutants of E. coli 536 has previously been described. The three mutants 536 PAI IΔ1, 536 PAI IΔ2 and 536 PAI IΔ3 as well as the subsequent mutants of 536 PAI IΔ2 were generated using lambda red homologous recombination by replacing the corresponding genomic region by a chloramphenicol acetyltransferase gene (cat) cassette. The cat cassette was flanked by FRT sites and amplified from vector pKD3 with primer pairs for the corresponding mutants. The resulting PCR products were purified and used for electroporation of competent E. coli strain 536/pKD46 cells cultivated at 30° C. in the presence of 100 mM arabinose to induce the γ, β and exo genes of the λ phage Red recombinase system. The correct exchange of each of the 536 PAI I fragments against the cat cassette was confirmed by PCR.

Bacterial Supernatants

Supernatants were prepared by growing 10⁸ CFU/ml bacteria in RPMI-1640 for 4 hours followed by centrifugation at 16,000 RPM for 10 minutes. Supernatants were filtered using a 0.2 μm filter before being added to cells.

Cell Culture

Human kidney carcinoma cells (A498), bladder carcinoma cells (HTB9), Burkitt lymphoma cells (Rajii and Daudi), hepatocellular carcinoma (HEP-G2), and mouse bladder cells (MB49) was cultured in RPMI-1640 supplemented with 10% heat-inactivated fetal calf serum (FCS) Colorectal adenocarcinoma cancer (DLD1) and lung carcinoma (A549) was cultured in RPMI-1640 supplemented with 5% FCS. Gastric adenocarcinoma (AGS) was cultured in F12K medium supplemented with 10% FCS. Cells were cultured at 37° C. with 5% CO₂.

In Vitro Infection, Bacterial Supernatant Treatment or Recombinant Protein Treatment

Cells were grown in 8-well chamber slides or 6-well plates overnight in appropriate media supplemented with FCS. Cells were washed, and serum free media was added prior to infection with appropriately diluted bacteria for 1 or 4 hours. For cells challenged with supernatants, cells were washed, and sterile bacterial supernatants were added to the cells for 4 hours. The challenged cells were analyzed by confocal microscopy, western blotting, qRT-PCR or whole genome transcriptomic analysis.

Divalent Ion Experiment

E. coli 536 was grown on TSA, harvested and washed with PBS. 10⁸ CFU/ml of bacteria was incubated with 100 μM of ZnCl₂, MgCl₂, MnCl₂ or CaCl₂) at 37° C. for 1 hour. Bacteria were then washed in PBS and appropriately diluted before adding to the cells.

Inhibitor Experiment

Cells were pre-treated with 50 μg/ml EGTA (Egtazic acid, Ca²⁺ chelator; Sigma) or 100 μg/ml IC261 (CK1α1 inhibitor; Sigma) for 30 minutes before bacteria infection or supernatant treatment.

Confocal Microscopy

Kidney epithelial cells were stained with rabbit anti-c-MYC (Abcam) or mouse anti-his6-tag primary antibodies (1:200 in 5% FBS/PBS, overnight at 4° C.), followed by secondary goat anti-rabbit Alexa Fluor 488-conjugated or goat-anti mouse Alexa Fluor 568-conjugated antibodies (Molecular Probes, A-11034) (1:200 in 5% FBS and 0.025% Triton X-100/PBS, 1 hour at room temperature). Cells were counterstained with DRAQ5 (Abcam) and examined using a LSM 510 META laser scanning confocal microscope (Carl Zeiss). Fluorescence signal was quantified in 50 cells per condition using Image J.

Co-Immunoprecipitation

Magnetic protein-G coupled dynabeads (Thermo Scientific) were washed in PBS, coated with antibodies directed to c-MYC (1:100, 30 minutes), incubated with recombinant c-MYC (500 ng, 30 minutes), and incubated with 50 μg of whole cell lysates, or with bacterial supernatants (1 hour). Proteins were eluted and prepared for western blot analysis.

Western Blotting

Cells were lysed with NP-40 lysis buffer supplemented with protease and phosphatase inhibitors (Roche diagnostics). Proteins were run on SDS-PAGE (4-12% Bis-Tris gels, Invitrogein), blotted onto PVDF membranes (Biorad), blocked and stained with primary rabbit anti-c-MYC (1:1000, Abcam), rabbit anti-CK1α1 (1:1000,) or rabbit-anti-Lon (1:2000, Biorbyt) overnight at 4° C. Membranes were washed and stained with HRP-conjucated goat anti-rabbit HRP conjugated secondary antibodies (1:4000, Cell Signaling) washed again, imaged using ECL plus detection reagent (GE Health Care) and quantified by ImageJ. β-actin was used as a loading control.

Whole Genome Transcriptomic Analysis

Total RNA was extracted from A498 cells using the RNeasy Mini Kit (Qiagen) and on-column DNase digestion. 100 ng of RNA was amplified using GeneChip 3′IVT PLUS Kit (ThermoFisher Scientific), then fragmented and labeled aRNA was hybridized onto Human Genome U219 or Mouse Genome MG430 array strips (16 hours at 45° C.), washed, stained and scanned in-house using the GeneAtlas system (Affymetrix). All samples passed the internal quality controls included in the array strips (signal intensity by signal to noise ratio; hybridization and labeling controls; sample quality by GAPDH signal and 3′-5′ ratio <3).

Data was normalized using Robust Multi Average implemented in the Partek Express Software (32, 33). Significantly altered genes were calculated by comparing cells treated with bacteria or bacterial supernatants to uninfected cells (PBS control) and sorted by relative expression (2-way ANOVA model using Method of Moments, absolute fold change >2.0) (34). Heat-maps were constructed using the free Gitools 2.1 software. Differentially expressed genes and regulated pathways were analyzed using the Ingenuity Pathway Analysis software (IPA, Ingenuity Systems, Qiagen).

Quantitative Real-Time (qRT)-PCR

Total RNA was reverse-transcribed using the SuperScript III First-Strand Synthesis System for RT-PCR (Invitrogen) and quantified in real time using QuantiTect SYBR Green-based PCR Kits for mRNA (QIAGEN) on a RotorGene Q cycler (QIAGEN). Human specific primers were described in Table 3. Quantitative RT-PCR (qRT-PCR) reactions were run in biological duplicates and triplicates, and gene expression was quantified using the 2^{-ΔΔ}CT method.

In-Gel Digestion and Mass Spectrometry

TCA precipitated supernatants from E. coli 536 and ΔPAI I was loaded on a NU-PAGE gel and stained with Coomassie blue. Five bands were excised from the E. coli 536 lane and five bands corresponding in size in the ΔPAI I lane. Bands were digested in trypsin followed by identification using liquid chromatography-tandem mass-spectrometry (LC-MS/MS). Analysis was performed on a tibrid mass spectrometer fusion (Thermo Fischer Scientific). Samples were injected and concentrated onto an Acclaim PepMap 100 C18 precolumn (75 μm×2 cm, Thermo Scientific, Waltham, Mass.) and separated on a Acclaim PepMap RSLC column (75 μm×25 cm, nanoViper, C18, 2 μm, 100 Å) at 40° C. Separation was performed at a flow rate of 300 nl/min in 135 min with a non-linear gradient ranging from 3% to 90% acetonitrile containing 0.1% formic acid. Raw data were analysed with Proteome Discoverer version 2.2 (Thermo Fisher Scientific). Peptides were identified using SEQUEST HT against UniProtKB EColi database (release 2018 Dec. 6). Precursor tolerance was set to 10 ppm and fragment tolerance was set to 0.05 ppm. Up to 2 missed cleavages were allowed and Percolator was used for peptide validation at a q-value of maximum 0.01.

Murine Urinary Tract Infection Model

Female C57BL/6 WT mice were used for experiments at 9-15 weeks of age, weighing 21-26 g. The mice were bred and housed in the specific pathogen-free MIG animal facilities (Lund, Sweden) with free access to food and water. Mice were intravesically infected with E. coli CFT073 or E. coli 536 under Isofluorane anesthesia (10⁸ CFU in 0.1 ml), through a soft polyethylene catheter (outer diameter 0.61 mm; Clay Adams). Animals were sacrificed under anesthesia by cervical dislocation at 24 hours and bladders were aseptically removed and frozen into cryomolds containing O.C.T. Urine counts were determined by growth on TSA plates (37° C., overnight) after serial dilutions. Neutrophils from uncentrifuged urine were quantified using a hemocytometer.

Bladder Cancer Model

C57BL/6 female mice were bred at the Department of Laboratory Medicine and used at ages from 7 to 12 weeks. For procedures, mice were anesthetized by intraperitoneal injection of a ketamine and xylazine cocktail. MB49 tumors were established as described (25). On day 0, the bladder was emptied and preconditioned by intravesical instillation of 100 μl poly-L-lysine solution (0.1 mg/ml) through a soft polyethylene catheter (Clay Adams, Parsippany, N.J.) with an outer diameter of 0.61 mm for 30 minutes before MB49 tumor cells (2×10⁵ in 100 μl PBS) were instilled. On days 3, 5, 7, 9 and 11, 100 μl of E. coli 536 or ΔPAI I bacterial supernatant or PBS (sham-treated controls) were instilled. Mice remained under anesthesia on preheated blocks with the catheter in place to prolong tumor exposure to E. coli 536 or ΔPAI I bacterial supernatant (approximately 1 hour). Groups of 5-7 mice for each treatment and control were sacrificed at each time point, and bladders were imaged and processed for histology.

Histology and Immunohistochemistry

Bladders were embedded in O.C.T. compound (VWR), and 5-μm-thick fresh cryosections on positively-charged microscope slides (Superfrost/Plus; Thermo Fisher Scientific) were fixed with 4% paraformaldehyde or acetone-methanol (1:1 v/v). For hemotoxylin-eosin (H&E) staining, Richard-Allan Scientific Signature Series Hematoxylin 7211 and Eosin-Y 7111 (Thermo Fisher Scientific) were used to counterstain the tissue sections. Imaging was done with AX10 (Carl Zeiss). Cryosections were permeabilized (0.25% Triton X-100, 5% fetal calf serum/PBS) and incubated with primary anti-c-MYC antibody (1:500, 4° C., overnight, ab39688, Abcam). This was followed by staining with Alexa Fluor® 488-labeled secondary goat anti-rabbit antibodies (1:200, 1 hour, room temperature, A-11011, Molecular Probes). Tissues were counterstained with (1:1000, 15 minutes, room temperature, Abcam) and examined using an LSM 510 confocal microscope (Carl Zeiss). The quantification of images and fluorescence was performed in ImageJ.

Clinical Pilot Study of UTI Prone Patients

To characterize gene expression levels in UTI prone patients during an infection free interval, we sequenced peripheral blood RNA from 5 children with a history of acute pyelonephritis and x children with long-term ABU. RNA was obtained during an infection-free interval, a least one year after the last episode of acute pyelonephritis or a positive urine culture in the ABU group. The patients were followed for at least six years and their susceptibility pattern and history of infection was documented by a pediatric nephrologist (D Karpmann), at the Department of Pediatrics, Lund University Hospital. A diagnosis of APN was based on fever (>38.5° C.) with significant bacteriuria, C-reactive protein (CRP)>20 mg/I and no symptoms of other infections. A diagnosis of ABU was based on at least three consecutive urine cultures yielding the same bacterial strain (>10⁵ CFU/ml of urine) in a child with no symptoms of UTI and no CRP increase. The APN group had not developed ABU at any time during follow-up. Informed written consent was obtained from all participants or their parents/guardians. The study was approved by the Ethics Committee of the medical faculty, Lund University, Sweden (LU106-02, LU236-99).

RNA Sequencing

RNA was extracted using the QUiamp RNA blood mini kit (Qiagen) or the PAXgene™ Blood RNA tube and PAX gene blood RNA system. Quantity and quality was determined by Nanodrop and bioanalyzer before proceeding with RNA-Seq library preparation. The quality of the library was evaluated using the Agilent Technologies 2100 Bioanalyzer and a DNA 1000-kit. The quantity of adapter ligated fragments was determined by qPCR using the KAPA SYBR FAST library quantification kit for Illumina GA (KAPA Biosystems). A 10 μM solution of the sequencing library was used in the cluster generation on the Cluster Station (Illumina Inc.). Paired-end sequencing with 100 bases read length was performed using the Genome Analyzer_{IIx}, according to the manufacturer's protocols. Sequence reads were mapped using Tophat using default settings and reads were aligned to human reference genome Hg37.2.

Bioinformatic Analysis

Log₂ FC<0.8 was used as cut-off for bioinformatic analysis. Functional changes and activated canonical pathways were analyzed with using Ingenuity Pathways Analysis (IPA, Qiagen). Gene Set Enrichment Analysis (GSEA, Broad institute, Cambride, Mass.) was used to assess whether a defined set of genes showed statistically significant, concordant differences between the patient groups.

Quantification and Statistical Analysis

For data determined to follow a Gaussian distribution (D'Agostino & Pearson normality test), statistical differences were determined by two-tailed t-test or one-way ANOVA. For non-parametric data, Mann-Whitney U-tests and Kruskal-Wallis tests (Dunn's correction) was used. Statistical significance was determined by GraphPad (Prism v. 7) and significance was assigned at *P<0.05, **P<0.01 and ***P<0.001.

TABLE 1
Top 50 up-regulated transcripts in APN patients (⅔)

| | | log2 fold-change | | |
|---|---|---|---|---|
| Genes | Entrez Gene Name | APN6 | APN7 | APN10 |
| Immune responses/ | | | | |
| T cell signaling or function | | | | |
| DPP4 | Dipeptidyl-peptidase 4 | 8.55 | 8.08 | 3.88 |
| CD8A | CD8a molecule | 7.96 | 7.13 | 3.95 |
| CD28 | CD28 molecule | 6.86 | 7.33 | 3.94 |
| CD226 | CD226 molecule | 7.21 | 7.41 | 3.43 |
| CD247 | CD247 molecule | 7.10 | 7.22 | 3.51 |
| CCR7 | Chemokine (C—C motif) receptor 7 | 6.57 | 6.33 | 4.70 |
| GZMK | Granzyme K (granzyme 3; tryptase II) | 5.82 | 5.72 | 5.78 |
| PRF1 | Perforin 1 (pore forming protein) | 7.09 | 5.72 | 4.22 |
| IL7R | Interleukin 7 receptor | 6.38 | 5.65 | 4.98 |
| GZMB | Granzyme B (granzyme 2, cytotoxic T- | 6.62 | 5.70 | 4.61 |
| | lymphocyte-associated serine esterase 1) | | | |
| CD3D | CD3d molecule, delta (CD3-TCR complex) | 6.26 | 6.05 | 4.39 |
| Immune responses/ | | | | |
| B cell signaling or function | | | | |
| CD72 | CD72 molecule | 7.62 | 7.38 | 2.72 |
| FCRLA | Fc receptor-like A | 7.32 | 6.92 | 3.40 |
| BLK | B lymphoid tyrosine kinase | 6.93 | 6.40 | 3.92 |
| VPREB3 | Pre-B lymphocyte 3 | 6.67 | 6.93 | 3.53 |
| TNFRSF13C | Tumor necrosis factor receptor superfamily, | 6.45 | 6.39 | 4.25 |
| | member 13C | | | |
| FCRL5 | Fc receptor-like 5 | 6.58 | 7.01 | 3.33 |
| CD79A | CD79a molecule, immunoglobulin-associated | 5.97 | 5.99 | 4.67 |
| | alpha | | | |
| Immune responses/ | | | | |
| Other immune cells | | | | |
| MMD | Monocyte to macrophage differentiation- | 8.38 | 8.07 | 2.38 |
| | associated | | | |
| LILRA4 | Leukocyte immunoglobulin-like receptor, | 7.30 | 6.44 | 4.55 |
| | subfamily A (with TM domain), member 4 | | | |
| PPBP | Pro-platelet basic protein (chemokine | 7.88 | 8.19 | 2.19 |
| | (C—X—C motif) ligand 7) | | | |
| KLRB1 | Killer cell lectin-like receptor subfamily B, | 6.40 | 5.48 | 4.79 |
| | member 1 | | | |
| Transcriptional regulation | | | | |
| MYC | v-myc myelocytomatosis viral oncogene | 7.09 | 6.83 | 5.16 |
| | homolog (avian) | | | |
| SPIB | Spi-B transcription factor (Spi-1/PU.1 related) | 7.22 | 7.25 | 4.41 |
| BACH 2 | BTB and CNC homology 1, basic leucine | 7.18 | 6.83 | 3.87 |
| | zipper transcription factor 2 | | | |
| PAX5 | Paired box 5 | 6.89 | 6.53 | 4.05 |
| HABP4 | Hyaluronan binding protein 4 | 6.74 | 6.83 | 3.37 |
| HNRNPA1 | Heterogeneous nuclear ribonucleoprotein A1 | 6.50 | 6.15 | 4.44 |
| MAF | v-maf musculoaponeurotic fibrosarcoma | 6.85 | 7.32 | 2.73 |
| | oncogene homolog (avian) | | | |
| ZNF202 | Zinc finger protein 202 | 6.81 | 6.68 | 3.23 |
| Cell growth and proliferation, cellular | | | | |
| organization, cell-cell interactions | | | | |
| NOG | Noggin | 6.50 | 7.19 | 6.32 |
| VSIG1 | V-set and immunoglobulin domain containing 1 | 6.47 | 6.57 | 5.29 |
| GRAP | GRB2-related adaptor protein | 7.19 | 7.40 | 3.73 |
| GPA33 | Glycoprotein A33 (transmembrane) | 6.80 | 7.01 | 4.32 |
| SDPR | Serum deprivation response | 7.46 | 7.30 | 2.50 |
| MYO IE | Myosin IE | 7.30 | 6.77 | 3.00 |
| Biosynthesis/metabolism | | | | |
| EPHX2 | Epoxide hydrolase 2, cytoplasmic | 7.61 | 7.24 | 3.81 |
| CYP2U1 | Cytochrome P450, family 2, subfamily U, | 6.61 | 6.54 | 3.56 |
| | polypeptide 1 | | | |
| Membrane functions | | | | |
| a) Lipid metabolism/receptors | | | | |
| OSBPL10 | Oxysterol binding protein-like 10 | 7.78 | 7.73 | 4.32 |
| b) ion channels | | | | |
| CLIC3 | Chloride intracellular channel 3 | 7.86 | 6.69 | 4.42 |
| Enzymes/ | | | | |
| Posttranslational modifications | | | | |
| NT5E | 5′-nucleotidase, ecto (CD73) | 7.08 | 6.71 | 5.77 |
| DPH1 | DPH1 homolog (S. cerevisiae) | 7.59 | 6.78 | 3.69 |
| GIMAP7 | GTPase, IMAP family member 7 | 6.49 | 6.65 | 4.44 |
| TBC1D9 | TBC1 domain family, member 9 (with GRAM | 8.14 | 7.44 | 1.36 |
| | domain) | | | |
| Unknown functions | | | | |
| SPATA20 | Spermatogenesis associated 20 | 6.80 | 5.83 | 5.60 |
| SAMD3 | Sterile alpha motif domain containing 3 | 6.66 | 6.06 | 5.15 |
| OAF | OAF homolog (Drosophila) | 7.73 | 7.78 | 2.11 |
| MTSS1 | Metastasis suppressor 1 | 7.38 | 7.07 | 2.86 |
| RPUSD2 | RNA pseudouridylate synthase domain | 6.70 | 6.35 | 4.07 |
| | containing 2 | | | |
| C12orf57 | Chromosome 12 open reading frame 57 | 6.46 | 6.60 | 4.05 |
| TRIM 58 | Tripartite motif containing 58 | 7.50 | 6.98 | 2.30 |

TABLE 2
Transcription regulators activated in APN patients

| | APN6 | | APN7 | | APN10 | |
|---|---|---|---|---|---|---|
| Transcription | Regulation | p-value of | Regulation | p-value of | Regulation | p-value of |
| Regulator | z-score | overlap | z-score | overlap | z-score | overlap |
| MYCN | 4.703 | 5.66E−13 | 4.575 | 3.85E−13 | 5.127 | 1.16E−15 |
| MYC | 4.423 | 9.19E−04 | 3.934 | 3.77E−04 | 4.087 | 5.45E−09 |
| KLF2 | 3.128 | 2.11E−01 | 2.599 | 3.69E−01 | 3.527 | 4.10E−01 |
| NCOR2 | 2.033 | 2.78E−01 | — | | 2.029 | 6.25E−02 |
| WT1 | 2.209 | 9.06E−02 | — | | — | — |
| ESR1 | 2.006 | 5.13E−01 | — | | — | — |
| CIITA | — | — | 2.31 | 1.41E−01 | — | |
| CUX1 | — | — | 2.068 | 1.06E−01 | — | — |

TABLE 3
qRT-PCR primers

| Gene | Primer sequence |
|---|---|
| c-MYB | Fwd 5′-ATTGTGGACCAGACCAGACC-3′ |
| | Rev 5′-GCTGGTGAGGCACTTTCTTC-3′ |
| c-MYC | QuantiTect c-MYC Primer Assay |
| | (Qiagen catalog # QT00035406) |
| CSNK1A1 | Fwd 5′-ATGGCGAGTAGCAGCGGCTC-3′ |
| | Rev 5′-CGTATGTGGGGGATGCCAAC-3′ |
| CEBPD | Fwd 5′-AGAAGTTGGTGGAGCTGTCG-3′ |
| | Rev 5′-CAGCTGCTTGAAGAACTGCC-3′ |
| MAX | Fwd 5′-GTTTCAATCTGCGGCTGACA-3′ |
| | Rev 5′-GAAGAGCATTCTGCCGCTTG-3′ |
| GAPDH | QuantiTect GAPDH Primer Assay |
| | (Qiagen catalog # QT00079247) |

### Example 5

Rapidly c-MYC Depletion in Response to Infection

Epithelial cells are targeted when bacteria ascend into the renal pelvis and cause acute pyelonephritis. While investigating molecular mechanisms of this disease, we discovered that epithelial c-MYC levels are modified by bacterial infection. A rapid reduction in c-MYC protein levels was detected following in vitro infection with uropathogenic E. coli (CFT073 and 536) (FIG. 15a-c). An asymptomatic bacteriuria (ABU) strain, in contrast, did not affect c-MYC levels, suggesting that MYC inhibition might be virulence-related. This was confirmed using a well-characterized collection of E. coli isolates from the urinary tract^{12,13}. Cellular c-MYC levels were reduced by 72% of the uropathogenic strains (n=36), compared to 30% of the ABU strains (n=20, P<0.001) (FIG. 15d). Corresponding cell viability data is shown in FIG. 21.

c-MYC Inhibition

To address the human relevance of these findings, MYC expression was quantified in a population-based study of febrile urinary tract infection in infants and young children. A diagnosis of acute pyelonephritis was based on a positive urine culture, a temperature >38° C. and a positive DMSA scan, confirming renal involvement. RNA was obtained from peripheral blood at the time of diagnosis and six months later, at the follow up visit after the completion of antibiotic therapy (FIG. 15e). MYC mRNA levels were markedly reduced during acute infection compared to the follow up samples (FIG. 15f-h). The in vitro findings and clinical data suggested that c-MYC is inhibited during infection with uropathogenic E. coli.

Gene Deletion Strategy to Identify Bacterial c-MYC Regulators/Inhibitors

To identify mechanism(s) of bacterial c-MYC inhibition, we selected the uropathogenic strain E. coli 536^{14,15}, in which virulence genes and chromosomal pathogenicity islands (PAIs) have been extensively characterized (FIG. 16a) E. coli 536 infection reduced MYC levels in human kidney cells to virtually undetectable levels after four hours (FIG. 16b-c, FIG. 22). A chromosomal region responsible for c-MYC suppression was identified, by screening specific pathogenicity island deletion mutants (ΔPAI I, ΔPAI II, ΔPAI I+II, ΔPAI III and ΔPAI IV). E. coli PAI I₅₃₆ (536-114) showed a “loss of function” phenotype, suggesting that regulators of c-MYC inhibition are encoded on this island (FIG. 16b-c). Specific deletions of known virulence genes encoded on PAI I did not affect the inhibitory phenotype, except for the 536Δhly1 mutant, which showed a partial phenotype (FIG. 16d). A hly2 deletion on PAI II also showed reduced inhibitory activity (FIG. 16d).

Sequential deletions from the 5′ end of PAI I were generated using lambda red homologous recombination²⁰ (FIG. 16e, f, Table 7) and deletion mutants were tested for effects on cellular c-MYC levels. Genes regulating c-MYC inhibition were localized to a 41,516-44,899 bp fragment (PAI I Δ2, ECP_3798-ECP_3829 FIG. 16f). Further deletions identified the active PAI I 2-5.3 sub-fragment (ECP_3821-ECP-3825, FIG. 16g), which encodes a bacterial ABC transporter complex, responsible for ATP-dependent uptake of metal ions¹⁶. c-MYC inhibition was significantly reduced in cells infected with the E. coli PAI I₅₃₆ Δ2-5.3 deletion mutant. Importantly, the effect on MYC activity was reproduced by deletion of the corresponding ABC transporter in E. coli CFT073, which in this strain is located outside of PAI I (FIG. 16h). ZnCl₂ pretreatment of E. coli 536 (100 μM, 30 min) reversed c-MYC inhibition suggesting that Zn²⁺ fluxes regulated by the bacterial ABC transporter affect the production of the bacterial MYC inhibitor. MgCl₂, CaCl₂) or MnCl₂ had no effect (FIG. 16i). ZnCl₂ pre-treatment did not affect bacterial growth (FIG. 23). Furthermore, c-MYC expression was inhibited by the E. coli 536 supernatant but not by PAI I₅₃₆ supernatant, suggesting that the bacterial effector is secreted (FIG. 16j, k).

Mechanism(s) of MYC Degradation in Host Cells

Transcription factors are rapidly degraded in host cells and c-MYC has an estimated half-life of around 30 minutes¹⁷. The reduction in c-MYC protein levels suggested that bacterial effector molecules may engage and degrade c-MYC or accelerate endogenous mechanisms of c-MYC protein degradation. To identify bacterial effectors of MYC inhibition, bacterial supernatants were subjected to mass spectrometry. The Lon protease was identified as an abundant constituent of active supernatants from E. coli 536 compared to inactive supernatants from E. coli ΔPAI I₅₃₆ (FIG. 17a, FIG. 24). Recombinant Lon E. coli protease was subsequently shown to degrade recombinant c-MYC in vitro, in a time dependent manner (FIG. 17b). Peptide fragments, identified by LS-MS/MS, revealed a loss of sequences corresponding to the MYC DNA-binding motif (M1-D118), the serine rich domain (G166-5276) and parts of the MAX-binding domain (K421-A439), (FIG. 17c, FIG. 25).

The bacterial Lon protease has been shown to target serine rich proteins under conditions of stress, suggesting that a high serine content may define the affinity of Lon for c-MYC (serine content 14.3%). A direct interaction of c-MYC with recombinant Lon protease was verified by co-immunoprecipitation of E. coli 536 supernatants, using c-MYC-coated Protein G-coupled dynabeads. Supernatants from E. coli 536 Δlon were negative (FIG. 17d, e).

Uptake of the recombinant Lon protease by human kidney cells was detected by confocal microscopy and the increase in cytoplasmic Lon staining was accompanied by a dose-dependent decrease in c-MYC staining (FIG. 17f). Furthermore, c-MYC inhibition was significantly attenuated (50-60%) in cells infected with the E. coli 536 Δhly^{1/2} and Δlon deletion mutant (FIG. 17g, h).

Weak pull-down of recombinant MAX (MYC associated factor X) was observed but degradation of MAX by Lon was not detected in vitro. Upstream regulators of c-MYC such as AKT, CDK9, CBEB1 were not degraded by Lon in vitro nor were housekeeping proteins HH3, Actin and GRP75 (FIG. 17i, FIG. 24). The results identify the bacterial Lon protease as a c-MYC inhibitor with direct effects on human cells.

Serine content (% total amino acids) of proteins in FIG. 17i is shown in the following table.

TABLE 4

| Protein | Serine content (%) |
|---|---|
| c-MYC | 14.4% |
| MAX | 14% |
| C/EBP-δ | 6% |
| C-MYB | 8.5% |
| AKT | 4.8% |
| CDK9 | 5.1% |
| CPEB1 | 12.9% |
| GRP75 | 6% |
| Actin | 6% |
| HH3 | 3.5% |

Hly Dependent Activation of CK1α1 and MYC Degradation

A second mechanism of c-MYC degradation was identified by gene expression analysis of infected cells. The kinase CK1α1 phosphorylates c-MYC at Serine²⁵², marking the protein for proteasomal degradation¹⁸. E. coli 536 increased CSNK1A1 expression and the kinase was activated in infected cells. By co-immunoprecipitation, binding of c-MYC to CK1α1 was detected in infected cell lysates and pharmacologic inhibition of CK1α1 with IC261 reduced c-MYC degradation¹⁹, linking CK1α1 activation to MYC degradation (FIG. 26). CK1α1 activation was attributed to hemolysin-induced Ca²⁺ fluxes²⁰ and the E. coli 536 Δhly^{1/2} double deletion mutant failed to activate CK1α1, further supporting this hypothesis (FIG. 26). Zn²⁺ fluxes control hemolysin expression^{21,22}, linking Hly and MYC degradation to the bacterial ABC transporter, located upstream of the hly I operon (FIG. 16i-l). While suggestive and quite likely, the precise mechanism of action has not been definitively demonstrated to be operating through the ABC transporter.

The results suggest that c-MYC is degraded in infected cells by direct Lon protease cleavage and by an alpha-hemolysin induced loop of CK1α1 activation.

Bacterial Inhibition of MYC Expression

c-MYC works in concert with a large number of transcriptional regulators and c-MYB acts as a transcriptional enhancer, trans-activating the MYC promoter by binding distal upstream regulatory regions^{23,24}. Transcriptomic analysis identified a network of differentially regulated MYB-related genes in infected cells (E. coli 536 vs. PAI I₅₃₆ FIG. 18a-b). MYB expression was not directly affected but c-MYB was degraded in a CK1α1 dependent manner (FIG. 18c-d). CEBPD²⁵ (CCAAT enhancer binding protein delta) was transcriptionally repressed by E. coli 536 infection (FIG. 18e-f). C/EBP-δ interacts with E2F1/RB complexes, inhibiting DNA-binding and the translation of genes, including c-MYC²⁶. C/EBP-δ protein levels were reduced four hours after E. coli 536 infection or exposure to the E. coli 536 supernatant. CEBPD was not regulated in a Lon and hemolysin dependent manner (FIG. 29). In contrast to MYC, MAX expression was not significantly affected by in vitro infection (FIG. 18g-i, 2 FIG. 29). Evidence of MYB suppression was further obtained in the clinical study of acute pyelonephritis (FIG. 18j). Systemic CEBPD levels were upregulated (P<0.05).

The results suggest that in addition to accelerating c-MYC degradation, bacteria inhibit c-MYC expression by targeting transcriptional c-MYC regulators. The identified mechanisms of c-MYC degradation/inhibition are summarized in FIG. 18k. The findings do not exclude additional bacterial effects on MYC and MYC-related genes, as redundancy may be expected to occur.

Broad c-MYC Suppression in Cancer Cells from Different Tissues

The MYC family of oncogenes is subject to “a bewildering assortment of genetic rearrangements” associated with many different tumors cell types²⁷. To understand if bacterial c-MYC suppression is generally relevant, tumor cell lines from different tissues were screened. E. coli 536 infection reduced cellular c-MYC levels in Burkitt lymphoma cells, where c-MYC was first discovered¹⁷, as well as gastric (AGS) and hepatic (HEP-G2) carcinoma cells (FIG. 19a). E. coli 536 supernatants also reduced MYC levels in human bladder (HTB9), small airway (A549), as well as murine bladder cells (MB49), (FIG. 19b). Supernatants from 536 Δlon or 536 Δhly^{1/2} were inefficient c-MYC inhibitors and E. coli PAI I₅₃₆ was inactive, confirming the involvement of the Lon protease and hemolysin in the MYC degradation process (FIG. 30). Cytotoxic effects of the Lon protease were not detected in human kidney cells, using the LDH, ATP and Prestoblue assays (FIG. 30).

Therapeutic Efficacy of the Lon Protease

Therapeutic efficacy of the bacterial c-MYC inhibitors was first examined in a murine bladder cancer model. Bladder cancer is common and among the most costly cancer forms, due to high recurrence rates and a lack of curative therapies^{28,29,30,36}. Bladder cancer was established in C57BL/6 mice by intra-vesical instillation of rapidly growing MB49 tumor cells (day 0, FIG. 19c). The treatment groups received recombinant Lon protease (0.1 ml, 250 μg/mL) or E. coli 536 supernatants intra-vesically on days 3, 5, 7, 9 and 11. The sham group received PBS and E. coli PAI I₅₃₆ supernatant was used as a negative control (FIG. 31). Tumor progression was assessed by bladder size and weight and tumor sizes were defined in H&E stained whole bladder mounts. The mice were sacrificed on day 12, when large tumors filled the bladder lumen of the sham group. A marked reduction in bladder size, weight and tumor size was recorded in the treatment groups (FIG. 19d-f). The effects were accompanied by a reduction in c-MYC tissue levels (FIG. 19g, FIG. 31-33). A strong reduction in gene expression and a return towards a healthier phenotype was observed in Lon treated mice. Cancer gene networks were strongly affected, comprising 96.6% of all regulated genes in whole bladder RNA extracts from Lon treated mice compared to the sham group (FIG. 19h-k). This included a subset of bladder cancer related genes, which were inhibited (11.8%). Central cancer biofunctions were markedly attenuated, specifically MYC expression and MYC-dependent gene networks While the treatment effects were shared between Lon and the E. coli 536 supernatant, Lon treatment was more efficient, defined by tumor progression and effects on gene expression. Genes potentially involved in toxicity were less strongly expressed in Lon-treated than in E. coli 536-treated mice (FIG. 33).

Therapeutic efficacy of the Lon protease was further demonstrated in the murine Apc^{Min/+} model of colon cancer. The model mimics human disease, especially familial polyposis where APC mutations increase the colon cancer susceptibility³¹. The treatment groups received recombinant Lon protease by oral gavage, twice daily for 14 days and the sham group received PBS (FIG. 20a). Tumor development was evaluated at sacrifice on day 15, by counting the number of polyps in intestinal segments and by measuring lesion size. Lon protease treatment reduced the polyp number compared to the sham group and compared to pre-treatment samples (FIG. 20b-d), suggesting that growth might have been prevented. A strong reduction in gene expression and a return towards a healthier phenotype was observed in Lon treated mice (cut off fold change >2, P<0.05, FIG. 20e-g). Cancer gene networks were strongly affected (93% of all regulated genes) including a subset of colorectal cancer genes (54%). The total number of regulated genes was markedly reduced and cancer biofunctions were attenuated in Lon treated mice compared to the sham group. MYC expression and MYC-dependent gene networks were inhibited (FIG. 20h). The effects of the Lon protease were partially reproduced in mice treated with the E. coli 536 supernatants (FIG. 34). MYC dependent genes were strongly suppressed in these mice.

Long-Term Follow Up and Toxicity

Long-term efficacy of Lon treatment was further investigated by follow up after the end of treatment on day 12, when the sham group was sacrificed. Lon treated mice remained asymptomatic until day 27, when hematuria was detected in 3/15 mice and the group was sacrificed. Bladder tumors were present in 15/15 mice but bladder weights and pathology scores remained lower than in the sham group (FIG. 35). In a separate experiment, Lon treatment was continued by intra-vesical Lon administration twice a week. The mice remained asymptomatic until day 34, when 2/12 mice developed hematuria and the group was sacrificed. The two symptomatic mice carried large tumors but in the remaining mice, bladder size, weight and pathology remained unchanged compared to day 12, suggesting a lack of progression. Gene expression remained strongly inhibited in these mice (cut off fold change ≥2, P<0.05, 95% compared to the sham group), suggesting a lasting therapeutic effect. Specifically, molecular mechanisms of cancer genes were strongly inhibited long-term (FIG. 35).

The APC^{Min+/-} mice were also followed long-term after the end of treatment, without further intervention. The sham group developed symptoms on days 119-175 (weight loss, restricted movement) but the Lon treated mice remained asymptomatic until day 175 (survival curve in FIG. 20i). Harvested tissues showed a significant reduction in intestinal polyp number and an increase in body weight compared to the sham group (FIG. 20j-k). Gene expression remained inhibited compared to the sham group (cut off fold change >2, P<0.05), (FIG. 20l-m, FIG. 36), suggesting a lasting effect. Cancer related genes were inhibited, as well as a MYC-dependent gene network. One mouse in the Lon-treated group was sacrificed on day 161, due to an unrelated condition.

Three approaches were taken to address if the Lon protease causes significant toxicity. First, the Lon concentrations that inhibited c-MYC in vitro were not found to reduce human kidney cell viability, suggesting that the decrease in c-MYC levels is not due to cell death (FIG. 30). Secondly, per-oral or intra-vesical Lon challenge of healthy C57BL/6 WT mice did not cause weight loss, behavioral change or gross pathology in the bladder or intestinal tract, compared to untreated mice (FIG. 37, 38). Interestingly, in healthy mice MYC expression was not strongly affected and few MYC-related genes were regulated, suggesting that Lon mainly targets tissues where MYC is overexpressed, such as cancer. Thirdly, in tumor bearing mice, protection was associated with a strong inhibition of overall gene expression, especially cancer genes (FIG. 19h, 6f). Except for those genes, very few additional genes were specifically regulated by Lon, supporting a loss of disease without a major gain of toxicity.

TABLE 5
Comparison of E. coli 536 supernatant and rLon
with classical bladder cancer therapeutics

| | Bladder size | | Bladder weight | |
|---|---|---|---|---|
| Treatment | % Sham (range) | | g ⁻¹ (range) | |
| Sham | 100 | (61-149)^{a} | 10.7 | (8.2-13.4)^{a} |
| n = 17 | | | | |
| 536 SN | 54 | (43-68)^{a} | 5.5 | (3.9-6.6)^{a} |
| n = 14 | | | | |
| rLon | 69 | (53-85)^{a} | 5.3 | (3.9-6.3)^{a} |
| n = 7 | | | | |
| Mitomycin | 67 | (54-87)^{b} | 4 | (3.4-5.9)^{b} |
| 25 μg n = 6 | | | | |
| Epirubicin | 65 | (53-70)^{b} | 3.9 | (3.2-4.8)^{b} |

25 μg n = 6

Materials and Methods for Example 5

Patients

Total RNA from peripheral blood was obtained from a prospective analytical cohort study at the KK Woman and Children's hospital in Singapore. Patients were enrolled randomly after written consent was taken in accordance to requirements of Institution ethics committee. The enrolled patients were children aged >1 months with a clinical UTI diagnosis based on fever without known source, pyuria and bacteriuria (>10⁴ CFU/ml for catheterized urine, or >10⁵ CFU/ml in mid-stream clean-catch urine). Exclusion criteria were neonates and those with atypical presentation of recurrent UTI, underlying renal or urological abnormalities. Peripheral blood samples were taken at the time of infection and at a six-month follow up visit.

Transcriptome and Pathway Analysis

The transcriptomic profile was analyzed in a subset of the enrolled patients with confirmed APN (n=18). Total RNA was purified from peripheral blood collected in Tempus blood RNA tubes (Applied Biosystems) and subjected to gene expression measurements using Affymetrix GeneChip Human Gene 2.1 ST Arrays. All arrays were quality controlled by using Normalized Unscaled Standard Errors (NUSE⁴¹). Fold Change was calculated by comparing intraindividual samples during APN and at six-month follow-up. Significantly altered genes were sorted by relative expression (2-way ANOVA model using Method of Moments, P-values <0.05 and absolute fold change >1.41) and analyzed using gene set enrichment analysis (GSEA).

Quantitative Real-Time (qRT)-PCR

Total RNA was reverse-transcribed using the SuperScript III First-Strand Synthesis System for RT-PCR (Invitrogen) and quantified in real time using QuantiTect SYBR Green-based PCR Kits for mRNA (QIAGEN) on a RotorGene Q cycler (QIAGEN). Human specific primers are described in table 7 below. Quantitative RT-PCR (qRT-PCR) reactions were run in biological duplicates.

TABLE 6
qRT-PCR primers

| Gene | Primer sequence |
|---|---|
| MYB | Fwd 5′-ATTGTGGACCAGACCAGACC-3′ |
| | Rev 5′-GCTGGTGAGGCACTTTCTTC-3′ |
| MYC | QuantiTect c-MYC Primer Assay |
| | (Qiagen catalog # QT00035406) |
| CSNK1A1 | Fwd 5′-ATGGCGAGTAGCAGCGGCTC-3′ |
| | Rev 5′-CGTATGTGGGGGATGCCAAC-3′ |
| CEBPD | Fwd 5′-AGAAGTTGGTGGAGCTGTCG-3′ |
| | Rev 5′-CAGCTGCTTGAAGAACTGCC-3′ |
| MAX | Fwd 5′-GTTTCAATCTGCGGCTGACA-3′ |
| | Rev 5′-GAAGAGCATTCTGCCGCTTG-3′ |
| GAPDH | QuantiTect GAPDH Primer Assay |
| | (Qiagen catalog # QT00079247) |

Bacterial Strains

APN (n=36) isolates and ABU (n=20) isolates were isolated during a prospective study of childhood UTI in Gothenburg, Sweden^{12,13}. The prototype strains E. coli CFT073 (O6:K2:H1)⁴², E. coli 536 (O6:K15:H31)¹⁵ and E. coli 83972 (OR:K5:H-)⁴³ were used as control strains. Bacteria was grown on trypsin soy agar (TSA) plates at 37° C. for 16 hours and was harvested in phosphate buffered saline (PBS, pH 7.2) and appropriately diluted for infection.

Sub-Cloning of E. coli ΔPAI I₅₃₆

The complete PAI deletion mutants of E. coli strain 536 have previously been described⁴⁴. The three mutants PAI I₅₃₆Δ1, PAI I₅₃₆Δ2 and PAI I₅₃₆Δ3 which lack different regions of PAI I₅₃₆ as well as the subsequent deletion of selected genes included within region 2 of PAI I₅₃₆ were generated using lambda Red-based homologous recombination by replacing the corresponding genomic region or genes by a chloramphenicol acetyltransferase gene (cat) cassette. The cat cassette was flanked by FRT sites and amplified from vector pKD3 with primer pairs for the corresponding mutants. The resulting PCR products were purified and used for electroporation of competent E. coli strain 536/pKD46 cells cultivated at 30° C. in the presence of 100 mM arabinose to induce the γ, β and exo genes of the λ phage Red recombinase system. The correct exchange of each of the PAI I₅₃₆ fragments against the cat cassette was confirmed by PCR. Deleted genes and ORFs are shown in the following table.

TABLE 7
Deleted genes/ORFs encoded on PAI I₅₃₆.
(Supplementary information for FIG. 16).

| PAI I | | Gene/ | |
|---|---|---|---|
| deletion | ORF's | ORF | |
| mutant * | Deleted | designation | Function |
| 1 | ECP_3765- | | |
| | ECP_3796 | | |
| 2 | ECP_3798- | | |
| | ECP_3829 | | |
| 3 | ECP_3830- | | |
| | ECP_3863 | | |
| 2-1 | ECP_3798- | ECP_3798 | Inovirus GP2 family protein |
| | ECP_3801 | ECP_3799 | Hypothetical protein |
| | | ECP_3800 | AlpA family transcriptional regulator |
| | | ECP_3801 | Hypothetical protein |
| 2-2 | ECP_3802- | ECP_3804 | IS110 family transposase (frame shifted) |
| | ECP_3809 | ECP_3805 | IS110 family transposase (frame shifted) |
| | | ECP_3806 | IS3 family transposase (frame shifted) |
| | | ECP_3807 | IS3 family transposase (frame shifted) |
| | | ECP_3808 | IS4 like element ISSfl9 family transposase (frame shifted) |
| | | ECP_3809 | Hypothetical protein |
| 2-3 | ECP_3810- | ECP_3810 | AraC family transcriptional regulator |
| | ECP_3817 | ECP_3811 | CS12 fimbriae major subunit |
| | | ECP_3812 | CS12 fimbriae periplasmic chaperone |
| | | ECP_3813 | CS12 fimbriae chaperone protein |
| | | ECP_3814 | CS12 outer membrane usher protein |
| | | ECP_3815 | CS12 fimbriae chaperone protein |
| | | ECP_3816 | CS12 fimbriae minor subunit protein precursor |
| | | ECP_3817 | CS12 fimbriae adhesin protein |
| 2-4 | ECP_3818- | ECP_3818 | IS110 family transposase |
| | ECP_3821 | ECP_3819 | Hypothetical proten |
| | | ECP_3820 | Helix-turn-helix domain containing protein |
| | | ECP_3821 | Hypothetical protein |
| 2-5 | ECP_3821 | ECP_3821 | Hypothetical protein |
| | (partial)- | ECP_3822 | Putative ABC metal-type transporter ATP-binding protein |
| | ECP3825 | ECP_3823 | Putative ABC metal-type transporter permease |
| | | ECP_3824 | Putative ABC metal-type substrate binding protein |
| | | ECP_3825 | Hypothetical protein |
| hly I^{§} | ECP_3826- | ECP_3826 | Alpha-hemolysin-activating lysine-acyltransferase HlyC |
| | ECP3829 | ECP_3827 | RTX toxin alpha-hemolysin HlyA |
| | | ECP_3828 | Type I secretion system permease/ATPase HlyB |
| | | ECP_3829 | Alpha-hemolysin TISS ABC transporter subunit HlyD |

*Mutants in FIG. 16e-g
^{§}Mutant with partial effect in FIG. 16d.

Bacterial Supernatants

Supernatants were prepared by growing 10⁸ CFU/ml bacteria in RPMI-1640 for 4 hours followed by centrifugation at 16,000 RPM for 10 minutes. Supernatants were filtered using a 0.2 μm filter before being added to cells.

Cell Culture

Human kidney carcinoma cells (A498), bladder carcinoma cells (HTB9), Burkitt lymphoma cells (Rajii and Daudi), hepatocellular carcinoma (HEP-G2), and mouse bladder cells (MB49) was cultured in RPMI-1640 supplemented with 10% heat-inactivated fetal calf serum (FCS) Colorectal adenocarcinoma cancer (DLD1) and lung carcinoma (A549) was cultured in RPMI-1640 supplemented with 5% FCS. Gastric adenocarcinoma (AGS) was cultured in F12K medium supplemented with 10% FCS. Cells were cultured at 37° C. with 5% CO₂.

In Vitro Infection, Bacterial Supernatant Treatment or Recombinant Protein Treatment

Cells were grown in 8-well chamber slides or 6-well plates overnight in appropriate media supplemented with FCS. Cells were washed, and serum free media was added prior to infection with appropriately diluted bacteria for 1 or 4 hours. For cells challenged with supernatants, cells were washed, and sterile bacterial supernatants were added to the cells for 4 hours. The challenged cells were analyzed by confocal microscopy, Western blotting, qRT-PCR or whole genome transcriptomic analysis.

Divalent Ion Experiment

E. coli 536 was grown on TSA, harvested and washed with PBS. 10⁸ CFU/ml of bacteria was incubated with 100 μM of ZnCl₂, MgCl₂, MnCl₂ or CaCl₂) at 37° C. for 1 hour. Bacteria were then washed in PBS and appropriately diluted before adding to the cells.

Inhibitor Experiment

Cells were pre-treated with 50 μg/ml EGTA (Egtazic acid, Ca²⁺ chelator; Sigma) or 100 μg/ml IC261 (CK1α1 inhibitor; Sigma) for 30 minutes before bacterial infection or supernatant treatment.

Confocal Microscopy

Kidney epithelial cells were stained with rabbit anti-c-MYC (Abcam), rabbit anti-CK1α1 (Cell Signaling Technology), rabbit anti-c-MYB (Sigma Aldrich), rabbit anti-CEBPD (Abcam) or mouse anti-his6-tag primary antibodies (1:200 in 5% FBS/PBS, overnight at 4° C.), followed by secondary goat anti-rabbit Alexa Fluor 488-conjugated or goat-anti mouse Alexa Fluor 568-conjugated antibodies (Molecular Probes, A-11034) (1:200 in 5% FBS and 0.025% Triton X-100/PBS, 1 hour at room temperature). Cells were counterstained with DRAQ5 (Abcam) and examined using a LSM 510 META laser scanning confocal microscope (Carl Zeiss). Fluorescence signal was quantified in 50 cells per condition using Image J.

Co-Immunoprecipitation

Magnetic protein-G coupled dynabeads (Thermo Scientific) were washed in PBS, coated with antibodies directed to GST or c-MYC (1:100, 30 minutes), incubated with recombinant c-MYC or MAX protein (500 ng, 30 minutes), and incubated with whole cell lysates (50 μg), bacterial supernatants, or recombinant Lon protease (0.2 μg) for 1 hour. Proteins were eluted and prepared for western blot analysis.

Western Blotting

Cells were lysed with NP-40 lysis buffer supplemented with protease and phosphatase inhibitors (Roche diagnostics). Proteins were run on SDS-PAGE (4-12% Bis-Tris gels, Invitrogen), blotted onto PVDF membranes (Biorad), blocked and stained with primary rabbit anti-c-MYC (1:1000, Abcam), rabbit anti-CK1α1 (1:1000, Cell Signaling Technology) or rabbit-anti-Lon (1:2000, Biorbyt) overnight at 4° C. Membranes were washed and stained with HRP-conjugated goat anti-rabbit HRP conjugated secondary antibodies (1:4000, Cell Signaling) washed again, imaged using ECL plus detection reagent (GE Healthcare) and quantified by ImageJ. β-actin was used as a loading control.

Cell Toxicity Assays

Cells (4×10⁴ cells/well) were seeded in serum-free RPMI-1640 on 96-well plates (Tecan Group Ltd). Cells were treated with recombinant Lon protease and incubated for four hours. The cell viability or cell death was quantified by three different assays. The released ATP was determined by the luminescence-based ATP Lite™ kit (Perkin Elmer). The mitochondrial enzymes activity was determined by the Presto Blue assay (Invitrogen, A13262). The lactate dehydrogenase (LDH) released from damaged cells were quantified by LDH assay (ThermoScientific, 88953). Assays were repeated twice in triplicate and mean value was calculated.

In-Gel Digestion and Mass Spectrometry

TCA precipitated supernatants from E. coli 536 and PAI I₅₃₆ was loaded on a NU-PAGE gel and stained with Coomassie blue. Five bands were excised from the E. coli 536 lane and five bands corresponding in size in the E. coli PAI I₅₃₆ lane. Bands were digested in trypsin followed by identification using liquid chromatography-tandem mass-spectrometry (LC-MS/MS). Analysis was performed on a tibrid mass spectrometer fusion (Thermo Fischer Scientific). Samples were injected and concentrated onto an Acclaim PepMap 100 C18 precolumn (75 μm×2 cm, Thermo Scientific, Waltham, Mass.) and separated on a Acclaim PepMap RSLC column (75 μm×25 cm, nanoViper, C18, 2 μm, 100 Å) at 40° C. Separation was performed at a flow rate of 300 nl/minute in 135 minutes with a non-linear gradient ranging from 3% to 90% acetonitrile containing 0.1% formic acid. Raw data were analyzed with Proteome Discoverer version 2.2 (Thermo Fisher Scientific). Peptides were identified using SEQUEST HT against UniProtKB EColi database (release 2018 Dec. 6). Precursor tolerance was set to 10 ppm and fragment tolerance was set to 0.05 ppm. Up to 2 missed cleavages were allowed and Percolator was used for peptide validation at a q-value of maximum 0.01.

In Vitro Degradation Assay

0.1 μg of recombinant proteins were incubated with 0.2 μg of Lon protease (Abcam, ab141921), or PBS, in a reaction buffer consisting of 20 mM MgCl₂ in the presence of ATP (2 mM) in 50 mM Tris. pH 8.0. Proteins were incubated at 37° C. for 60, 120 or 240 minutes and were denatured using 10 mM DTT in NuPAGE Sample Buffer at 100° C. for 5 minutes before further analysis.

Identification of c-MYC Cleavage by Lon Protease

Recombinant c-MYC protein 1 μg was incubated with 0.2 μg Lon protease or PBS for 4 hours at 37° C. Samples were diluted with NuPAGE LDS sample buffer (Invitrogen) mixed with 10 mM dithiotreitol (DTT) and incubated at 56° C. for 20 min followed by addition of iodoacetamide (IAA) to a final concentration of 20 mM. Samples were loaded on 4-12% NuPAGE (Invitrogen). The electrophoresis was run in MOPS buffer. The gel was Coomassie stained and bands of interest were excised from the SDS-PAGE and trypsin digested, followed by identification using LC-MS/MS as per above. MS/MS spectra were investigated with PEAKS (version 10) against UniProt Human (released 20200221) where the sequence for GST-tag-MYC had been added. The precursor tolerance and fragment tolerance were set to 10 ppm and 0.05 Da, respectively. None enzyme was selected, methionine oxidation and deamidation of asparagine and glutamine were treated as dynamic modification and carbamidomethylation of cysteine as a fixed modification.

Whole Genome Transcriptomic Analysis

Total RNA was extracted from A498 cells or whole bladder or intestinal tissue from mice using the RNeasy Mini Kit (Qiagen) and on-column DNase digestion. 100 ng of RNA was amplified using GeneChip 3′IVT PLUS Kit (ThermoFisher Scientific), then fragmented and labeled aRNA was hybridized onto Human Genome U219 or Mouse Genome MG430 array strips (16 hours at 45° C.), washed, stained and scanned in-house using the GeneAtlas system (Affymetrix). All samples passed the internal quality controls included in the array strips (signal intensity by signal to noise ratio; hybridization and labeling controls; sample quality by GAPDH signal and 3′-5′ ratio <3). Data was normalized using Robust Multi Average implemented in the Partek Express Software^{45,46.} Significantly altered genes were calculated by comparing cells or tissue infected with bacteria to uninfected controls (PBS control) and sorted by relative expression (2-way ANOVA model using Method of Moments, absolute fold change >2.0)⁴⁷.

Bladder Cancer Model

C57BL/6J female mice were bred at the Department of Laboratory Medicine and used at ages from 7 to 12 weeks. For procedures, mice were anesthetized by intraperitoneal injection of a ketamine and xylazine cocktail. MB49 tumors were established as described⁴⁸. On day 0, the bladder was emptied and preconditioned by intravesical instillation of 100 μl poly-L-lysine solution (0.1 mg/ml) through a soft polyethylene catheter (Clay Adams, Parsippany, N.J.) with an outer diameter of 0.61 mm for 30 minutes before MB49 tumor cells (2×10⁵ in 100 μl PBS) were instilled. On days 3, 5, 7, 9 and 11, 100 μl of E. coli 536 or PAI I₅₃₆ bacterial supernatant, Lon protease (250 μg/ml) or PBS (sham controls) were instilled. Mice remained under anesthesia on preheated blocks with the catheter in place to prolong tumor exposure to E. coli 536 or PAI I₅₃₆ bacterial supernatant (approximately 1 hour). Lon protease with an addition of a 6×HIS-tag, was commercially produced in the E. coli K12 strain with endotoxin levels <1.0 EU/μg. Groups of 5-7 mice for each treatment and control were sacrificed at each time point, and bladders were imaged and processed for histology. For long-term follow up experiments, mice were treated for 12 days with Lon protease (250 μg/ml) or received PBS (Sham) and were left until they developed signs of hematuria. Alternatively, Lon treatment (250 μg/ml) was administered twice weekly until mice developed signs of hematuria.

Colon Cancer Model

C57BL/6J-APC^{Min}/J that develop spontaneous intestinal adenoma formation³¹ were bred at the Department of Laboratory medicine and used at either the age of 11 weeks, or 20 weeks. The mice were treated twice daily for 14 days using oral gavage (200 μl) of E. coli 536 WT supernatants (10 μg/kg) or using recombinant Lon Protease (250 μg/ml). At sacrifice, intestines were aseptically harvested and opened longitudinally, the number of polyps were counted in a blinded manner by two independent researchers. Different segments of the intestine were then prepared for either Swizz-rolls, protein extraction or mRNA extraction for further analysis. For long-term follow-up experiments, mice were treated per-orally, twice daily for 14 days with Lon protease (250 μg/ml) or received PBS (Sham) and left until mice developed reduced mobility or significant weight loss.

Methylene Blue Staining

The opened intestinal segments were spread flat between sheets of filter paper and fixed overnight in 10% neutral buffered formalin. Formalin-fixed sections were transferred to 70% ethanol and stained with 0.2% methylene blue. Stained sections were rinsed in deionized water and imaged by dissecting microscope.

Histology and Immunohistochemistry

Bladders were embedded in O.C.T. compound (VWR), and 5-μm-thick fresh cryosections on positively-charged microscope slides (Superfrost/Plus; Thermo Fisher Scientific) were fixed with 4% paraformaldehyde or acetone-methanol (1:1 v/v). Swiss rolls were fixed in 4% paraformaldehyde and then embedded in paraffin and sectioned. For hemotoxylin-eosin (H&E) staining, Richard-Allan Scientific Signature Series Hematoxylin 7211 and Eosin-Y 7111 (Thermo Fisher Scientific) were used to counterstain the tissue sections. Imaging was done with AX10 (Carl Zeiss). Sections were permeabilized (0.25% Triton X-100, 5% fetal calf serum/PBS) and incubated with primary anti-c-MYC antibody (1:500, 4° C., overnight, ab39688, Abcam). This was followed by staining with Alexa Fluor® 488-labeled secondary goat anti-rabbit antibodies (1:200, 1 hour, room temperature, A-11011, Molecular Probes). Tissues were counterstained with (1:1000, 15 minutes, room temperature, Abcam) and examined using an LSM 510 confocal microscope (Carl Zeiss). The quantification of images and fluorescence was performed in ImageJ.

Quantification and Statistical Analysis

For data determined to follow a Gaussian distribution (D'Agostino & Pearson normality test), statistical differences were determined by two-tailed t-test or one-way ANOVA. For non-parametric data, Mann-Whitney U-tests were used. Paired data was analyzed by Wilcoxon matched pairs signed rank test. Kaplan Mayer plots were analysed using the log-rank Mantel-Cox test. Statistical significance was determined by GraphPad (Prism v. 7) and significance was assigned at *P<0.05, **P<0.01 and ***P<0.001.

Discussion

Microbes are essential constituents of most natural environments and potent regulators of tissue homeostasis and health in human hosts. Viruses enter host cells and reprogram the cellular machinery for their own benefit but bacteria often have a different strategy. As independent cells, they do not need invasion to survive or establish contact with host tissues, but can send messengers that trigger desired host cell effects. It is increasingly being realized that the messengers are not just the classical exotoxins or innate immune activators, with destructive effects, but molecules that reprogram basic cellular functions by targeting key functional elements, such as the transcriptional machinery^{32,33}. This study identifies the pleiotropic transcription factor and oncogene c-MYC as a specific bacterial target. We show that uropathogenic E. coli infection depletes c-MYC from infected tissues and explain this effect by accelerated c-MYC protein degradation and attenuated MYC expression. The bacterial strategy for c-MYC inhibition is also shown to translate to cancer therapy, as the bacterial Lon protease responsible for MYC degradation attenuates cancer progression and increases survival. The bacterial Lon protease thus shows significant promise as a new approach to targeting MYC therapeutically.

The Lon protease is a conserved, ATP-dependent peptidase that maintains homeostasis in prokaryotes by processing short-lived regulatory proteins³⁴. The protease was shown to degrade c-MYC in a time-dependent manner. Lon is a serine protease and serine-rich repeats in c-MYC were identified as cleavage sites by mass spectrometry. The c-MYC cleavage products had lost essential functional domains, such as the DNA-binding and C terminal MAX-binding sites. MYB expression was not directly affected but c-MYB was degraded in a CK1α1 dependent manner. MAX was affected, however, suggesting an unexpected level of specificity. The lower sensitivity to Lon degradation of MAX and upstream regulators of c-MYC may reflect their lower serine content. This specificity of Lon for c-MYC may be critical to limit the effects of c-MYC inhibition and avoid causing toxicity or a general metabolic collapse that would be expected to accompany a MYC shut-down³⁵. Except for modest effects on MYC-dependent cellular functions, no evidence of major Lon toxicity was observed, supporting a loss of disease without a major gain of toxicity. This should be addressed further in extended studies.

The results further suggest that pathogenic bacteria regulate cellular MYC levels through a mechanism involving the pore forming toxin hemolysin (ref.). Ca²⁺ fluxes, which are activated by hemolysin, have previously been shown to activate CK1α1 and an E. coli 536 hly^{1/2} deletion mutant had lost the ability to activate CK1α1, indicating a clear role for hemolysin in CK1α1 activation. CK1α1 was also involved in MYB degradation, predicted to further attenuate MYC expression. Hemolysin was not required for Lon to be active, however, as the recombinant Lon protein alone inhibited MYC in human cells and treated tumors. It is therefore reasonable to assume that the effects of the two MYC inhibition mechanisms might be additive In tissues exposed to bacterial strains that express both Lon and Hly. The localized effects in tissues adjacent to the site of infection further suggest that it may be possible to achieve local MYC inhibition, at specific tissue sites.

Myc inhibition has convincingly been shown to arrest cancer progression and to restore tissue integrity in transgenic models^{35,36}. This study suggests that the Lon protease holds promise as a broadly applicable anti-tumor agent. While the precise mechanism of action for Lon protease in vivo remains to be conclusively defined, gene expression analysis identified striking changes in treated tissues. The overexpression of MYC and cancer genes was reversed in most mice, resulting in disease attenuation in two models. Importantly, this response was not observed in healthy mice treated with Lon, suggesting that the MYC overexpression phenotype may define the therapeutic window of this molecule. Clearly, per-oral administration was effective, despite the complexity of the intestinal microbiota and tumor progression was delayed long-term in both models.

It is not clear why oncogenic mutations become fixed in the population, as cancer is a disease mainly of post reproductive age groups. There are good arguments that, at least in humans, cancer is not much of a selective force. With tragic exceptions, cancer is a disease of advanced age, and the incidence increases by a power of 4 or 5, as the reproductive value declines^{37,38}. Stated another way, beneficial genes that act late in life, like those that may reduce the incidence of cancers, would provide little advantage. Severe, life-threatening infections, in contrast, are a strong selective force. Prior to the era of antibiotics, the mortality of acute pyelonephritis was around 15% and we are now seeing the re-emergence of these severe disease consequences. As MYC drives renal development and acute pyelonephritis is a significant cause of renal growth retardation^{39,40}, MYC overexpression may have evolved to protect local host environments against detrimental effects of pathogenic bacteria. Without being bound by theory, it may be speculated that by inhibiting mucosal c-MYC expression, the bacteria may inadvertently provide a defense against oncogenic transformation later in life.
1 Beutler, B. Innate immunity: an overview. Molecular Immunology 40, 845-859, doi:10.1016/j.molimm.2003.10.005 (2004).
2 Ferrandon, D., Imler, J. L., Hetru, C. & Hoffmann, J. A. The Drosophila systemic immune response: sensing and signalling during bacterial and fungal infections. Nat Rev Immunol 7, 862-874, doi:10.1038/nri2194 (2007).
3 Kumar, H., Kawai, T. & Akira, S. Pathogen Recognition by the Innate Immune System. International Reviews of Immunology 30, 16-34, doi:10.3109/08830185.2010.529976 (2011).
4 Dang, C. V. MYC on the Path to Cancer. Cell 149, 22-35, doi:10.1016/j.cell.2012.03.003 (2012).
5 Meyer, N. & Penn, L. Z. MYC—TIMELINE Reflecting on 25 years with MYC. Nature Reviews Cancer 8, 976-990, doi:10.1038/nrc2231 (2008).
6 Nilsson, J. A. & Cleveland, J. L. Myc pathways provoking cell suicide and cancer. Oncogene 22, 9007-9021, doi:10.1038/sj.onc.1207261 (2003).
7 Wierstra, I. & Alves, J. Cyclin E/Cdk2, P/CAF, and E1A regulate the transactivation of the c-myc promoter by FOXM1. Biochemical and Biophysical Research Communications 368, 107-115, doi:10.1016/j.bbrc.2008.01.039 (2008).
8 Chen, H., Liu, H. D. & Qing, G. L. Targeting oncogenic Myc as a strategy for cancer treatment. Signal Transduction and Targeted Therapy 3, doi:UNSP 5 10.1038/s41392-018-0008-7 (2018).
9 Caforio, M. et al. Recent advances in searching c-Myc transcriptional cofactors during tumorigenesis. Journal of Experimental & Clinical Cancer Research 37, 239, doi: 10.1186/s13046-018-0912-2 (2018).
10 Hann, S. R. MYC cofactors: molecular switches controlling diverse biological outcomes. Cold Spring Harbor Perspectives in Medicine 4, doi:10.1101/cshperspect.a014399 (2014).
11 Mugrauer, G. & Ekblom, P. Contrasting Expression Patterns of 3 Members of the Myc Family of Protooncogenes in the Developing and Adult-Mouse Kidney. Journal of Cell Biology 112, 13-25, doi:DOI 10.1083/jcb.112.1.13 (1991).
12 Caugant, D. A. et al. Genetic Diversity in Relation to Serotype in Escherichia-Coli. Infection and Immunity 49, 407-413, doi:Doi 10.1128/Iai.49.2.407-413.1985 (1985).
13 Leffler, H. & Svanborgeden, C. Glycolipid Receptors for Uropathogenic Escherichia-Coli on Human-Erythrocytes and Uroepithelial Cells. Infection and Immunity 34, 920-929, doi:Doi 10.1128/Iai.34.3.920-929.1981 (1981).
14 Dobrindt, U. et al. S-fimbria-encoding determinant sfa(I) is located on pathogenicity island 111536 of uropathogenic Escherichia coli strain 536. Infection and Immunity 69, 4248-4256, doi:Doi 10.1128/Iai.69.7.4248-4256.2001 (2001).
15 Middendorf, B. et al. Instability of pathogenicity islands in uropathogenic Escherichia coli 536. Journal of Bacteriology 186, 3086-3096, doi:10.1128/Jb.186.10.3086-3096.2004 (2004).
16 Consortium, T. U. <https://www.uniprot.org/uniprot/A0A454A9A9> (2019).
17 Ramsay, G., Evan, G. I. & Bishop, J. M. The Protein Encoded by the Human Proto-Oncogene C-Myc. Proceedings of the National Academy of Sciences of the United States of America—Biological Sciences 81, 7742-7746, doi:DOI 10.1073/pnas.81.24.7742 (1984).
18 Li, L. K. et al. GLIPR1 Suppresses Prostate Cancer Development through Targeted Oncoprotein Destruction. Cancer Research 71, 7694-7704, doi:10.1158/0008-5472.Can-11-1714 (2011).
19 Mashhoon, N. et al. Crystal structure of a conformation-selective casein kinase-1 inhibitor. Journal of Biological Chemistry 275, 20052-20060, doi:DOI 10.1074/jbc.M001713200 (2000).
20 Uhlen, P. et al. alpha-Haemolysin of uropathogenic E-coli induces Ca2+ oscillations in renal epithelial cells. Nature 405, 694-697 (2000).
21 Patzer, S. I. & Hantke, K. The ZnuABC high-affinity zinc uptake system and its regulator Zur in Escherichia coli. Molecular Microbiology 28, 1199-1210, doi:10.1046/j.1365-2958.1998.00883.x (1998).
22 Yatsunyk, L. A. et al. Structure and metal binding properties of ZnuA, a periplasmic zinc transporter from Escherichia coli. J Biol Inorg Chem 13, 271-288, doi:10.1007/s00775-007-0320-0 (2008).
23 Cogswell, J. P. et al. Mechanism of C-Myc Regulation by C-Myb in Different Cell Lineages. Molecular and Cellular Biology 13, 2858-2869, doi:Doi 10.1128/Mcb.13.5.2858 (1993).
24 Nakagoshi, H., Kaneiishii, C., Sawazaki, T., Mizuguchi, G. & Ishii, S. Transcriptional Activation of the C-Myc Gene by the C-Myb and B-Myb Gene-Products. Oncogene 7, 1233-1240 (1992).
25 Balamurugan, K. & Sterneck, E. The many faces of C/EBPdelta and their relevance for inflammation and cancer. Int J Biol Sci 9, 917-933, doi:10.7150/ijbs.7224 (2013).
26 Gery, S., Tanosaki, S., Hofmann, W. K., Koppel, A. & Koeffler, H. P. C/EBPdelta expression in a BCR-ABL-positive cell line induces growth arrest and myeloid differentiation. Oncogene 24, 1589-1597, doi:10.1038/sj.onc.1208393 (2005).
27 Whitfield, J. R., Beaulieu, M. E. & Soucek, L. Strategies to Inhibit Myc and Their Clinical Applicability. Front Cell Dev Biol 5, 10, doi: 10.3389/fcell.2017.00010 (2017).
28 Siegel, R. L., Miller, K. D. & Jemal, A. Cancer statistics, 2015. CA: a cancer journal for clinicians 65, 5-29 (2015).
29 Torre, L. A. et al. Global cancer statistics, 2012. CA: a cancer journal for clinicians 65, 87-108 (2015).
30 Vos, T. et al. Global, regional, and national incidence, prevalence, and years lived with disability for 328 diseases and injuries for 195 countries, 1990-2016: a systematic analysis for the Global Burden of Disease Study 2016. The Lancet 390, 1211-1259 (2017).
31 Ren, J., Sui, H., Fang, F., Li, Q. & Li, B. The application of ApcMin/+ mouse model in colorectal tumor researches. Journal of Cancer Research and Clinical Oncology 145, 1111-1122, doi:10.1007/s00432-019-02883-6 (2019).
32 Ambite, I. et al. Fimbriae reprogram host gene expression—Divergent effects of P and type 1 fimbriae. PLoS Pathog 15, e1007671, doi:10.1371/journal.ppat.1007671 (2019).
33 Lutay, N. et al. Bacterial control of host gene expression through RNA polymerase II. J Clin Invest 123, 2366-2379, doi:10.1172/JCI66451 (2013).
34 Lee, I. & Suzuki, C. K. Functional mechanics of the ATP-dependent Lon protease-lessons from endogenous protein and synthetic peptide substrates. Biochimica Et Biophysica Acta—Proteins and Proteomics 1784, 727-735, doi:10.1016/j.bbapap.2008.02.010 (2008).
35 Pelengaris, S., Khan, M. & Evan, G. c-MYC: More than just a matter of life and death. Nature Reviews Cancer 2, 764-776, doi:10.1038/nrc904 (2002).
36 Soucek, L. et al. Modelling Myc inhibition as a cancer therapy. Nature 455, 679-683, doi:10.1038/nature07260 (2008).
37 Yancik, R. et al. Effect of age and comorbidity in postmenopausal breast cancer patients aged 55 years and older. JAMA 285, 885-892, doi:10.1001/jama.285.7.885 (2001).
38 Yancik, R. et al. Comorbidity and age as predictors of risk for early mortality of male and female colon carcinoma patients: a population-based study. Cancer 82, 2123-2134 (1998).
39 Baker, L. R. I., Cattell, W. R., Fry, I. K. F. & Mallinson, W. J. W. Acute-Renal-Failure Due to Bacterial Pyelonephritis. Quarterly Journal of Medicine 48, 603-612 (1979).
40 Oeyen, S. et al. Long-term outcome after acute kidney injury in critically-ill patients. Acta Clinica Belgica 62, 337-340, doi:DOI 10.1179/acb.2007.076 (2007).
41 Leek, J. T. & Storey, J. D. Capturing heterogeneity in gene expression studies by surrogate variable analysis. Plos Genetics 3, 1724-1735, doi:ARTN e161 10.1371/journal.pgen.0030161 (2007).
42 Mobley, H. L. T. et al. Pyelonephritogenic Escherichia-Coli and Killing of Cultured Human Renal Proximal Tubular Epithelial-Cells—Role of Hemolysin in Some Strains. Infection and Immunity 58, 1281-1289, doi:Doi 10.1128/Iai.58.5.1281-1289.1990 (1990).
43 Zdziarski, J. et al. Host Imprints on Bacterial Genomes-Rapid, Divergent Evolution in Individual Patients. Plos Pathogens 6, doi:ARTN e1001078 10.1371/journal.ppat.1001078 (2010).
44 Datsenko, K. A. & Wanner, B. L. One-step inactivation of chromosomal genes in Escherichia coli K-12 using PCR products. Proceedings of the National Academy of Sciences of the United States of America 97, 6640-6645, doi:DOI 10.1073/pnas.120163297 (2000).
45 Bolstad, B. M., Irizarry, R. A., Astrand, M. & Speed, T. P. A comparison of normalization methods for high density oligonucleotide array data based on variance and bias. Bioinformatics 19, 185-193, doi:DOI 10.1093/bioinformatics/19.2.185 (2003).
46 Irizarry, R. A. et al. Exploration, normalization, and summaries of high density oligonucleotide array probe level data. Biostatistics 4, 249-264, doi:DOI 10.1093/biostatistics/4.2.249 (2003).
47 Eisenhart, C. The Assumptions Underlying the Analysis of Variance. Biometrics 3, 1-21, doi:Doi 10.2307/3001534 (1947).
48 Mossberg, A. K., Hou, Y. C., Svensson, M., Holmqvist, B. & Svanborg, C. HAMLET Treatment Delays Bladder Cancer Development. Journal of Urology 183, 1590-1597, doi:10.1016/j.juro.2009.12.008 (2010).

1. A therapeutic agent comprising Lon protease, or a variant or active fragment or equivalent thereof; alpha-hemolysin, or a variant or active fragment or equivalent thereof; CK1α1, or a variant or active fragment or equivalent thereof; a c-MYB inhibitor or a CEBP-δ inhibitor; or combination thereof, for use in therapy, with the proviso that the therapeutic agent does not comprise a bacteria or bacterial supernatant or lysate.

2. A therapeutic agent for use according to claim 1, wherein the Lon protease, alpha-hemolysin, c-MYB inhibitor and/or CEBP-δ inhibitor are bacterial, preferably wherein the bacteria is E. coli, more preferably E. coli 536 or CFT073.

3. A therapeutic agent for use according to any preceding claim, comprising isolated Lon protease, or a variant or active fragment or equivalent thereof, isolated alpha-hemolysin, or a variant or active fragment or equivalent thereof, isolated CK1α1, or a variant or active fragment or equivalent thereof, an isolated c-MYB inhibitor and/or an isolated CEBP-δ inhibitor.

4. A therapeutic agent for use according to any preceding claim, comprising synthetic Lon protease, or a variant or active fragment or equivalent thereof, synthetic alpha-hemolysin, or a variant or active fragment or equivalent thereof, synthetic CK1α1, or a variant or active fragment or equivalent thereof, a synthetic c-MYB inhibitor and/or a synthetic CEBP-δ inhibitor

5. A therapeutic agent for use according to any preceding claim, wherein the c-MYB inhibitor and/or CEBP-δ inhibitor are proteins or variants or active fragments thereof.

6. A therapeutic agent for use according to any preceding claim, wherein the use is treatment or prevention of a disease wherein c-MYC levels are elevated.

7. A therapeutic agent for use according to any preceding claim, wherein the use is treatment or prevention of cancer and/or infection, preferably wherein the cancer is lymphoma, epithelial cancer or mucosal cancer, preferably wherein the infection is of the urinary tract.

8. A therapeutic agent for use according to any preceding claim, wherein the c-MYB inhibitor and/or a CEBP-δ inhibitor are inhibitors of c-MYB and/or CEBP-δ expression.

9. A recombinant microorganism engineered to express elevated levels of Lon protease, or a variant or active fragment or equivalent thereof, alpha-hemolysin, or a variant or active fragment or equivalent thereof, CK1α1, or a variant or active fragment or equivalent thereof, a c-MYB inhibitor and/or a CEBP-δ inhibitor.

10. A microorganism extract obtained from a recombinant microorganism according to claim 10.

11. A method of obtaining isolated Lon protease, or a variant or active fragment or equivalent thereof, isolated alpha-hemolysin, or a variant or active fragment or equivalent thereof, isolated CK1α1, or a variant or active fragment or equivalent thereof, an isolated c-MYB inhibitor and/or an isolated CEBP-δ inhibitor, the method comprising isolating the Lon protease, or a variant or active fragment or equivalent thereof, the alpha-hemolysin, or a variant or active fragment or equivalent thereof, the CK1α1, or a variant or active fragment or equivalent thereof, the c-MYB inhibitor and/or the CEBP-δ inhibitor from the recombinant microorganism of claim 9 or the microorganism extract of claim 10.

12. A kit comprising isolated Lon protease, or a variant or active fragment or equivalent thereof, isolated alpha-hemolysin, or a variant or active fragment or equivalent thereof, isolated CK1α1, or a variant or active fragment or equivalent thereof, an isolated c-MYB inhibitor and/or an isolated CEBP-δ inhibitor.

13. A kit according to claim 12, comprising synthetic Lon protease, or a variant or active fragment or equivalent thereof, synthetic alpha-hemolysin, or a variant or active fragment or equivalent thereof, synthetic CK1α1, or a variant or active fragment or equivalent thereof, a synthetic c-MYB inhibitor and/or a synthetic CEBP-δ inhibitor.

14. A kit according to claim 12 or 13, wherein the Lon protease, or variant or active fragment or equivalent thereof, alpha-hemolysin, or variant or active fragment or equivalent thereof, CK1α1, or a variant or active fragment or equivalent thereof, c-MYB inhibitor and/or CEBP-δ inhibitor are in powder form.

15. A recombinant microorganism according to claim 9, a recombinant microorganism extract according to claim 10, or a kit according to any of claims 12-14, for use in therapy.

16. A pharmaceutical composition, comprising a therapeutic agent according to any of claims 1-8, a recombinant microorganism according to claim 9, or a microorganism extract according to claim 10, plus a pharmaceutically acceptable carrier, excipient and/or adjuvant.

17. A method of treating cancer and/or infection, the method comprising administering an effective amount of a therapeutic agent according to any of claims 1-8, a recombinant microorganism according to claim 9, a microorganism extract according to claim 10, or a pharmaceutical composition according to claim 16, to a subject in need thereof.
